(19) United States
(12) Patent Application Publication
(10) Pub. No.: US 2024/0219390 A1
(43) Pub. Date: Jul. 4, 2024

(54) CANCER BIOMARKERS

(71) Applicant: ELYPTA AB, Solna (SE)

(72) Inventors: Francesco GATTO, Stockholm (SE); Siniša BRATULIC, Göteborg (SE); Jens NIELSEN, Hellerup (DK); Andrea BACCONI, San Diego, CA (US)

(21) Appl. No.: 18/557,529

(22) PCT Filed: Apr. 28, 2022
(86) PCT No.: PCT/EP2022/061385

§ 371 Date: Oct. 26, 2023

Related U.S. Application Data
(60) Provisional application No. 63/180,768, filed on Apr. 28, 2021.

(30) Foreign Application Priority Data
Sep. 17, 2021 (GB) 2113334.3

(51) Int. Cl. G01N 33/574; G01N 33/66

(52) U.S. Cl. CPC G01N 33/57488; G01N 33/66; G01N 2400/40

(57) ABSTRACT

The present invention relates to a method of screening for cancer in a subject, said method comprising determining the level and/or chemical composition of the protein-free fraction of one or both of the glycosaminoglycans (GAGs) chondroitin sulfate (CS) and heparan sulfate (HS) in a body fluid sample, wherein said sample has been obtained from said subject.

The present invention relates to biomarkers for cancer and to methods of screening for cancer. Such methods involve determining the level and/or composition of certain biomarkers which are indicative of cancer in a subject.

The number of cancer cases is predicted to increase substantially in the near future. The rising cancer population determines an urgent need to improve the current diagnostics landscape for cancer. In particular, affordable and practical tools for cancer diagnostics are needed to assist healthcare professionals in the early detection of high risk cancer, which typically correlates with more favorable clinical outcomes, or to guide treatment of current cancer patients.

Circulating biomarkers are molecules that can be measured in accessible body fluids of individuals, e.g. blood or urine, and whose levels are useful to assist in the diagnosis and/or prognosis and/or prediction of response to treatment. An example of a widely used biomarker is the prostate-specific antigen (PSA) for prostate cancer, the carcinoembryonic antigen (CEA) for colorectal cancer and the carbohydrate antigen 125 for ovarian cancer. However, the clinical value of these biomarkers for diagnosing cancer is highly debated. For example, a standard PSA test to detect prostate adenocarcinoma in men over 50 years old at average risk, assuming a cut-off value equal to 4 ng/ml, has typical values for sensitivity and specificity equal to 21% (51% for high grade lesions with Gleason score greater or equal to 8) and 91%, respectively (Wolf, A. M., et al., 2010 C A Cancer J Clin 60, 70-98).

What is needed in the art are new methods of screening for cancer (e.g. diagnosing cancer). The identification of novel biomarkers for cancer may potentially have clinical implications for a large number of patients and would be an important clinical advancement. Here, the inventors provide evidence for a blood and/or urine marker of cancer which can be used in a highly specific (e.g. 98% specificity has been demonstrated) and sensitive assay to detect cancer. Advantageously therefore, such methods are non-invasive and performed on readily obtainable samples, as well as being highly accurate.

The availability of such tests also has value for a number of medical decisions, for example to determine the risk of progression in newly diagnosed cancers; to guide treatment options in cancer patients with uncertain clinical risk; to monitor cancer before and after surgery or drug treatment; to rule out the relapse of the disease during a longer period of time after which a patient is typically declared cured; to assess the occurrence of cancer in a population at risk, such as genetically predisposed individuals or individuals presenting risk factors or individuals presenting symptoms; to ascertain whether a metastasis is due to a particular cancer; to predict recurrence or relapse in patients with early stage cancer; to distinguish lesions suspicious of cancer from non-malignant diseases; to detect certain stages (e.g. early stage) or grades (e.g. low grade) of cancer; to determine the tissue of origin of a cancer; or to screen for cancer in the general population.

The present inventors have identified that the level of certain glycosaminoglycans (GAGs), in particular the level of the protein-free fraction of certain GAGs such as chondroitin sulfate (CS) and heparan sulfate (HS) and/or the chemical compositions of said GAGs, in particular the chemical composition of the protein-free fraction of said GAGs, are found at differential levels in body fluid samples from cancer patients in comparison to control subjects. These differential levels of the GAGs CS or HS, or differential chemical compositions of the GAGs CS or HS (GAG profiles), can act as biomarkers for cancer and thus are useful in screening for cancer in subjects. The present inventors have thus determined that GAG profiles from accessible fluids are suitable to be used as a biomarker/diagnostic marker of cancer.

Surprisingly and advantageously, the present inventors have found that changes in the level of the GAGs CS and/or HS, in particular where the protein-free fraction of the GAGs are analysed, are observed in accessible body fluids of cancer patients and that these GAG profiles are suitable to be used as a biomarker of cancer. The present inventors have also shown that in addition to the overall (total) levels or concentration of CS and/or HS, other changes in the chemical composition, for example the specific disaccharide sulfation patterns of CS and/or HS, in particular where the protein-free fraction of the GAGs are analysed, are also observed between cancer samples and normal samples and can be used very effectively to diagnose cancer.

Surprisingly, it had not before been appreciated that an analysis of the protein-free fraction of the GAGs as desribed herein, for example as opposed to the entire GAG population (which includes both protein-free and protein-bound GAGs), could give rise to an effective diagnosis of cancer. This finding by the present inventors advantageously gives rise to improvements and efficiencies in the GAG processing steps required for GAG-based cancer screening methods. For example, in preferred methods of the present invention, there is no need to contact the samples with a proteolytic agent (or other appropriate agent) in order to “release” or “free” or “convert” the protein-bound fraction of the GAGs to a protein-free (or more protein-free) fraction such that they can be analysed. Thus, in preferred methods of the present invention it is specifically the protein-free fraction (or naturally protein-free fraction) of the GAGs in the body fluid samples that are analysed.

The present inventors have also found that other improvements and efficiencies in the GAG processing steps can made. For example, it has surprisingly been found that methods in which the purification step in which GAGs in a sample are purified based on their negative charge, e.g. using methods such as anion-exchange chromatography or using an anion exchange resin, is omitted, result in improved processing and measurement of the GAGs, whether the protein-free or entire (protein-free plus protein-bound) fraction of GAGs is being analysed.

Clearly the finding that cancer diagnosis can be carried out in an accessible body fluid sample, e.g. blood or urine, from a subject is extremely advantageous. Here, the inventors have observed a systemic alteration of GAG composition that was concomitant with cancer.

Advantageously the present inventors have also shown that the identified markers that are distinctive of occurrence of cancer and that are calculated based on measurements in accessible body fluids are accurate and robust predictors of the disease.

Thus, in one aspect the present invention provides a method of screening for cancer in a subject, said method comprising determining the level and/or chemical composition of the protein-free fraction of one or both of the glycosaminoglycans (GAGs) chondroitin sulfate (CS) and heparan sulfate (HS) in a body fluid sample, wherein said sample has been obtained from said subject.

Preferred methods of the present invention provides a method of screening for a cancer selected from the group consisting of a genitourinary cancer (e.g. kidney cancer, prostate cancer or bladder cancer), a respiratory tract cancer (e.g. lung cancer), a brain tumor, a blood cancer (e.g. a lymphoma), colorectal cancer, uterine cancer, a gastrointestinal-neuroendocrine tumour, a breast cancer, ovarian cancer and head and neck cancer.

In some embodiments, the genitourinary cancers is kidney cancer (e.g. renal cell carcinoma), prostate cancer or bladder cancer.

In some embodiments, the respiratory tract cancer is a lung cancer or a head and neck cancer of the respiratory tract.

In some embodiments the brain tumor is a glioma (e.g. diffuse glioma).

In some embodiments, the blood cancer is a lymphoma.

In some embodiments, the breast cancer is a breast invasive ductal carcinoma.

In some embodiments, the uterine cancer is cervix squamous cell carcinoma or endometrial cancer.

In some embodiments, the head and neck cancer is head and neck squamous cell carcinoma.

In some embodiments, the prostate cancer may be prostate adenocarcinoma.

In some embodiments, the colorectal cancer may be colorectal carcinoma.

In some embodiments, the gastrointestinal-neuroendocrine tumour may be small intestinal neuroendocrine tumour.

In some embodiments, the endometrial cancer may be endometrial adenocarcinoma.

In some embodiments, the ovarian cancer may be ovarian epithelial carcinoma.

Other preferred methods of the present invention provides a method of screening for a cancer selected from the group consisting of: bladder cancer, breast invasive ductal carcinoma, cervix squamous cell carcinoma, chronic lymphoid leukaemia, colorectal cancer, endometrial cancer, diffuse glioma, a gastro-intestinal neuroendocrine tumour, head and neck squamous cell carcinoma, diffuse large B-cell lymphoma, non-small cell lung cancer, ovarian cancer, prostate cancer and renal cell cancer. Diffuse large B-cell lymphoma may also be referred to herein as non-follicular lymphoma.

References to “cancer” herein can refer to any type of cancer, or to one or more of the types of cancer set forth in the paragraphs above.

In some embodiments, a genitourinary cancer is not screened for. In some embodiments, a respiratory tract cancer is not screened for. In some embodiments, a brain tumor is not screened for. In some embodiments, a blood cancer is not screened for. In some embodiments, a genitourinary cancer is not screened for. In some embodiments, colorectal cancer is not screened for. In some embodiments, colorectal carcinoma is not screened for. In some embodiments, a uterine cancer is not screened for. In some embodiments, a gastro-intestinal endocrine tumour is not screened for. In some embodiments, a small intestinal endocrine tumour is not screened for. In some embodiments, breast cancer is not screened for. In some embodiments, head and neck cancer is not screened for. In some embodiments, ovarian cancer is not screened for. In some embodiments, ovarian epithelial carcinoma is not screened for. In some embodiments, renal cell cancer is not screened for. In some embodiments, prostate cancer is not screened for. In some embodiments, prostate adenocarcinoma is not screened for. In some embodiments, bladder cancer is not screened for In some embodiments, lung cancer is not screened for. In some embodiments, a glioma is not screened for. In some embodiments, a lymphoma is not screened for. In some embodiments, breast invasive ductal carcinoma is not screened for. In some embodiments, cervix squamous cell carcinoma is not screened for. In some embodiments, endometrial cancer is not screened for. In some embodiments, endometrial adenocarcinoma is not screened for. In some embodiments, chronic lymphoid leukaemia is not screened for. In some embodiments, diffuse glioma is not screened for. In some embodiments, head and neck squamous cell carcinoma is not screened for. In some embodiments, diffuse large B-cell lymphoma is not screened for. In some embodiments, non-small cell lung cancer is not screened for. In some embodiments, renal cell cancer (or renal cell carcinoma) is not screened for. In some embodiments, thyroid cancer is not screened for. In some embodiments, pancreatic cancer is not screened for. In some embodiments, liver cancer is not screened for. In some embodiments, bile duct cancer is not screened for. In some embodiments, stomach cancer is not screened for. In some embodiments, oesophageal cancer is not screened for. In some embodiments, skin cancer is not screened for. In some embodiments, melanoma is not screened for. In some embodiments, a neuroendocrine tumour is not screened for.

In some embodiments, one or more (or all) of a kidney cancer (e.g. renal cell cancer), a skin cancer (e.g. a melanoma) and prostate cancer are not screened for.

In preferred methods of the invention an altered level and/or chemical composition of chondroitin sulfate (CS) and/or heparan sulfate (HS) in said protein-free fraction in comparison to a control level and/or chemical composition is indicative of cancer in said subject.

In some embodiments of certain methods of the invention, an altered level and/or chemical composition of chondroitin sulfate (CS) and/or heparan sulfate (HS) in comparison to a control level and/or chemical composition is indicative of cancer in said subject.

In preferred methods of the invention both the level and the chemical composition are determined. In other preferred methods of the invention the chemical composition alone is determined, or, in other preferred methods, the level (total level or total concentration) of CS and/or HS alone is determined.

Methods of the present invention comprise determining the level and/or chemical composition of one or both of the glycosaminoglycans (GAGs) chondroitin sulfate (CS) and heparan sulfate (HS) in a body fluid sample. In some embodiments, the level and/or chemical composition of one of said GAGs is determined. In some embodiments, the level and/or chemical composition of chondroitin sulfate (CS) is determined. In some embodiments, the level and/or chemical composition of heparan sulfate (HS) is determined. In some embodiments, the level and/or chemical composition of chondroitin sulfate (CS) and heparan sulfate (HS) is determined.

In some preferred methods of the present invention, the method comprises determining the level and/or chemical composition of the protein-free fraction of one or both of the glycosaminoglycans (GAGs) chondroitin sulfate (CS) and heparan sulfate (HS) in a body fluid sample. Thus, in some embodiments, the level and/or chemical composition of the protein-free fraction of chondroitin sulfate (CS) is determined. In some embodiments, the level and/or chemical composition of the protein-free fraction of heparan sulfate (HS) is determined. In some embodiments, the level and/or chemical composition of the protein-free fraction of chondroitin sulfate (CS) and heparan sulfate (HS) is determined.

In some embodiments, the level and/or chemical composition of hyaluronic acid (HA) is additionally determined.

Glycosaminoglycans (GAGs) are sugar containing molecules which can be attached to proteins on serine residues, i.e. can form a part of a proteoglycan. They are formed from linear or unbranched chains of monosaccharides (i.e. are polysaccharides) which can be sulfated. Heparan sulfate (HS), chondroitin sulfate (CS), keratan sulfate (KS), hyaluronic acid (HA) and heparin are the common types of GAG, of which HS and CS are examples of sulfated GAGs. The different types of GAG are distinguished by different repeating disaccharide units.

When linked or attached to proteins, CS and HS are GAGs that share a common biosynthetic route in the linkage to the core protein, but thereafter they differ in their polymerisation in that the CS repeating disaccharide is made up of repeating N-acetylgalactosamine (GalNAc) and glucuronic acid residues (GlcA), whilst the repeating disaccharide in HS is typically made up of repeating N-acetylglucosamine (GlcNAc) and glucuronic acid (GlcA) residues. Each monosaccharide is attached by a specific enzyme allowing for multiple levels of regulation over GAG synthesis.

Although GAGs can be attached to proteins, i.e. they may be in a protein-bound or proteoglycan form, GAGs can also exist in a “free” form, i.e. they can also exist in a non protein-bound or non-proteoglycan form. Such “free” forms of GAGs are referred to herein as “protein-free GAGs”.

Thus, in body fluids (or body samples) there is typically a protein-free fraction of GAGs (or protein-free pool of GAGs) and a protein-bound fraction of GAGs (or protein-bound pool of GAGs). Together (i.e. protein-free fraction plus protein-bound fraction), the two fractions may be referred to as the entire GAG fraction or entire GAG pool.

As discussed elsewhere herein, in preferred methods of the invention, the level or composition of the protein-free fraction of one or both of the GAGs chondroitin sulfate (CS) and heparan sulfate (HS) in a body fluid sample is determined.

The protein-free fraction GAGs (or disaccharide units derived therefrom as discussed elsewhere herein) for analysis may be obtained by any suitable means.

As discussed elsewhere herein, body fluid samples are typically processed prior to analysis. Such processing typically comprises subjecting the GAGs to a processing step to obtain disaccharide units for analysis. Such a processing step typically comprises contacting said sample (or said GAGs in said sample) with an enzyme (e.g. a GAG lyase such as a chondroitinase or a heparinase) which digests (or fragments) the GAGs into disaccharide units. Without wishing to be bound by theory, such enzymes are not able to access protein-bound GAGs but rather act on (or use as their substrate) only (or essentially only) protein-free GAGs. If proteoglycans (which are proteins with GAGs bound or attached thereto) are contacted with a proteolytic agent (e.g. a protease such as proteinase K) the protein component thereof is digested by the proteolytic agent and the protein-bound GAGs are freed or released, meaning that protein-bound GAGs are (or are converted into) into a “free” or “released” form, which would then be available for digestion (or fragmentation) by an enzyme such as a GAG lyase. As in certain preferred methods of the invention it is specifically the level and/or composition of the protein-free fraction (or naturally protein-free fraction) of one or both of the GAGs CS and HS that is determined, then preferred methods of the invention do not comprise contacting the sample with a proteolytic agent (e.g. a protease such as proteinase K). Omitting a proteolytic agent during processing of a sample for analysis is thus a way to obtain (or obtain only or obtain essentially only) the protein-free fraction of GAGs (or disaccharides subsequently derived therefrom) for analysis. Protein-bound GAGs (proteoglycan GAGs) that have been (or become) “freed” or “released” by the action of a proteolytic agent (e.g. a protease) are not protein-free GAGs in accordance with the present invention.

Thus, protein-free GAGs (or the protein-free fraction of GAGs) are GAGs (or the fraction of GAGs) that are already (or naturally) free (i.e. not protein-bound) in the absence of (or without) the sample having been treated with a proteolytic agent (e.g. a protease). Put another way, the protein-free GAGs (or the protein-free fraction of GAGs) are GAGs (or the fraction of GAGs) that are free (i.e. not protein-bound) in an original, or initial, or unprocessed sample. For example, the protein-free GAGs (or the protein-free fraction of GAGs) can be GAGs (or the fraction of GAGs) that are present in a sample, e.g. an original or unprocessed sample, and are susceptible to, or accessible to, or available for (e.g. are a substrate for) digestion (or fragmentation) into disaccharide units as described elsewhere herein, e.g. using an enzyme such as a lyase enzyme.

As indicated above, protein-free GAGs (or the protein-free fraction of GAGs) may be considered the non-protein bound (or non-protein bound fraction) or non-proteoglycan form (or non-proteoglycan fraction) of GAGs. Put another way, the protein-free GAGs (or the protein-free fraction of GAGs) are GAGs not decorating a proteoglycan in the original, or initial, or unprocessed sample.

Thus, in some preferred embodiments, methods comprise determining the level and/or chemical composition of the protein-free fraction of one or both of the glycosaminoglycans (GAGs) chondroitin sulfate (CS) and heparan sulfate (HS) in a body fluid sample, wherein the sample is subjected to processing prior to determining said level and/or composition and wherein said processing does not comprise contacting said sample with a proteolytic agent (e.g. a protease such as proteinase K), or other agent which can release protein-free GAGs from protein-bound GAGs.

Alternatively viewed, in some preferred embodiments, methods comprise determining the level and/or chemical composition of the protein-free fraction of one or both of the glycosaminoglycans (GAGs) chondroitin sulfate (CS) and heparan sulfate (HS) in a body fluid sample, wherein the sample is subjected to processing prior to determining said level and/or composition and wherein said processing does not comprise contacting said sample with a proteolytic agent (e.g. a protease such as proteinase K), or other agent which can release GAGs (or GAG chains) from proteoglycans.

Thus, in some preferred embodiments, methods comprise determining the level and/or chemical composition of the protein-free fraction of one or both of the glycosaminoglycans (GAGs) chondroitin sulfate (CS) and heparan sulfate (HS) in a body fluid sample, wherein said sample has been obtained from said subject and has been subjected to processing prior to determining said level and/or chemical composition, wherein said processing
(a) comprises fragmenting said one or both GAGs into disaccharide units; and
(b) does not comprise prior to (a) contacting said sample with a proteolytic agent.

As indicated above, protein-bound GAGs (or the protein-bound fraction of GAGs) may be considered proteoglycan GAGs (or the proteoglycan fraction of GAGs). Alternatively viewed, protein-bound GAGs (or the protein-bound fraction of GAGs) may be considered as GAGs that typically require the protein to which they are bound to be contacted with a proteolytic agent (e.g. a protease such as a non-specific protease) in order for them to be (or become) freed or released.

In some other methods of the invention the level and/or chemical composition of the entire fraction (or entire pool) of one or both of the GAGs CS and HS in a body fluid sample may be determined (i.e. protein-free GAGs plus protein-bound GAGs). In such embodiments, the sample is typically contacted with a proteolytic agent during processing of the sample.

The “level” of HS or CS as referred to herein generally refers to the total level or amount (e.g. concentration) of the HS or CS present in the sample. The level of CS and/or HS in a sample can be measured or determined by any appropriate method which would be well-known and described in the art. Some methods involve electrophoresis, in particular capillary electrophoresis, e.g. capillary electrophoresis with fluorescence detection, e.g. laser-induced fluorescence detection. Other suitable methods are gel electrophoresis, e.g. agarose gel electrophoresis (e.g. FACE, fluorophore-assisted carbohydrate electrophoresis) or mass spectrometry or liquid chromatography, e.g. HPLC, optionally in combination with mass spectrometry (HPLC-MS). Preferred methods involve high performance liquid chromatography (HPLC), preferably ultra-HPLC (UHPLC), in combination with mass spectrometry, e.g. MS/MS or triple quadropole mass spectrometry. Preferred methods comprise ultra-high-performance liquid chromatography (UHPLC) coupled with electrospray ionization triple-quadrupole mass spectrometry system.

Conveniently these levels can be measured as a concentration (e.g. a real or absolute level or concentration), for example, as a number of microgram per ml (μg/ml). However, again, any appropriate measure of level may be used.

In the preferred methods of the present invention the levels of HS and/or CS, preferably the levels of the protein-free fraction of HS and/or CS, are determined separately or individually. In other words the methods do not involve the measurement of total GAG levels in a sample or the total levels of all the GAGs present in combination, e.g. in the protein-free GAG fraction, but involve the measurement of the levels of one or more of the individual GAGs HS or CS.

In particular embodiments, the level (e.g. total level, or concentration) of CS and/or HS, e.g. the level of the protein-free fraction of CS and/or HS, can be determined in, for example, blood or urine samples.

In some embodiments of the invention, an increased level (or concentration) of CS in a body fluid sample and/or an increased level (or concentration) of HS in a body fluid sample and/or an increased level (or concentration) is indicative of cancer in said subject and can be used to screen, diagnose, etc., subjects as described elsewhere herein.

In some embodiments of the invention, an increased level (or concentration) of CS in a blood (e.g. plasma) sample is indicative of a cancer selected from the group consisting of: uterine cancer (e.g. cervix squamous cell carcinoma or endometrial cancer), blood cancer (e.g. a lymphoma such as chronic lymphoid leukaemia), brain tumor (e.g. diffuse glioma), a gastro-intestinal endocrine tumour, head and neck cancer (e.g. head and neck squamous cell carcinoma), lung cancer (e.g. non-small cell lung cancer), ovarian cancer, bladder cancer, prostate cancer, and kidney cancer (e.g. renal cell cancer) in said subject and can be used to screen, diagnose, etc., subjects as described elsewhere herein.

In some embodiments, an increased level (or concentration) of CS in a urine sample is indicative of a cancer selected from the group consisting of: lung cancer (e.g. non-small cell lung cancer), head and neck cancer (e.g. head and neck squamous cell carcinoma), kidney cancer (e.g. renal cell cancer) and bladder cancer in said subject and can be used to screen, diagnose, etc., subjects as described elsewhere herein.

In some embodiments, an increased level (or concentration) of HS in a urine sample is indicative of a cancer selected from the group consisting of: lung cancer (e.g. non-small cell lung cancer), head and neck cancer (e.g. head and neck squamous cell carcinoma) and kidney cancer (e.g. renal cell cancer) in said subject and can be used to screen, diagnose, etc., subjects as described elsewhere herein.

The individual monosaccharide units making up the CS and HS can have different sulfation patterns in terms of the position of the sulfate molecules and the amount/number of sulfate molecules. For CS, sulfation may most commonly occur at one or more of position 2 of the GlcA and positions 4 and 6 of the GalNAc. For HS, sulfation may occur at one or more of position 2 of the GlcA after epimerization to IdoA (iduronic acid), positions 3 and 6 of the GlcNAc, and N-sulfation of the GlcNAc. Thus, each individual disaccharide in the GAG chain may have 0 (i.e. be unsulfated), 1, 2, 3 or 4 (only in HS) sulfation forms and this in turn gives rise to different overall chemical compositions of GAG chains in terms of sulfation levels and specific disaccharide sulfation patterns.

As described elsewhere herein, preferred embodiments of the invention involve the determination of the chemical composition of one or both of CS and HS, in particular the chemical composition of the protein-free fraction of one or both of CS and HS. The term “chemical composition” as used herein can refer to both the levels of the GAGs as well as the disaccharide sulfation composition of the GAGs. In particular, this term includes a determination of one or more particular forms, e.g. sulfation forms, of the disaccharides making up the CS or HS GAGs. Put another way, the term “chemical composition” refers to the amount or level of one or more of the various sulfated and/or unsulfated forms of CS or HS disaccharides, as well as, for example, some other properties of the individual GAGs present, such as total HS or CS GAG levels, or other properties related to GAG sulfation such as HS charge or CS charge as described further elsewhere herein. Such a chemical composition which is analysed or determined in the present invention can also be referred to herein as a GAG profile, GAG forms, GAG features, GAG properties, GAGome, GAGome features.

Thus, for example, the term “chemical composition” as used herein may refer to a determination or analysis of the sulfation patterns (e.g. one or more of the sulfation forms) of the disaccharides making up CS and/or HS.

For example, for CS, there are 8 main sulfated and unsulfated forms (sulfation patterns, disaccharide sulfation forms) which are: 0s CS (also referred to as unsulfated CS or CS O unit), 2s CS (also referred to as chondroitin-2-sulfate), 4s CS (also referred to as chondroitin-4-sulfate or CS A unit), 6s CS (also referred to as chondroitin-6-sulfate or CS C unit), 2s4s CS (also referred to as chondroitin-2-4-sulfate), 2s6s CS (also referred to as chondroitin-2-6-sulfate or CS D unit), 4s6s CS (also referred to as chondroitin-4-6-sulfate or CS E unit) and Tris CS (also referred to as chondroitin-2-4-6-sulfate or trisulfated CS).

Each of the above is a form of CS GAG (a CS GAG form or property) which may be measured in the methods of the present invention. One or more of these forms may be measured, for example up to 8, e.g. 1, 2, 3, 4, 5, 6, 7 or all 8 of these sulfation forms may be measured. In some embodiments, measurement of all 8 of these sulfation forms is preferred.

Another GAG property for CS which may be measured in the methods of the present invention is the total concentration of CS (also referred to herein as CS tot or Tot CS or Total CS) or the total level of CS. This is typically measured as a concentration, e.g. an absolute concentration, e.g. in μg/ml, as described elsewhere herein. In some embodiments, the total CS is measured by summing the level of all measured CS disaccharide forms listed above. In embodiments of the invention where the total concentration of CS is measured as one of the GAG properties, then it is preferred that at least one other GAG property or CS property is measured, e.g. a property that is not based on the total level of the other individual GAGs present (e.g. not total HS or total HA). In some embodiments the total concentration of CS is not measured. In some embodiments, the measurement of one or more CS GAG properties is preferred.

“Charge CS” is another GAG form or property which may be measured in the present invention, e.g. as part of the GAG profile. “Charge CS” refers to the total fraction of sulfated disaccharides of CS, i.e. the fraction of sulfated disaccharides of CS present or measured in a sample out of the total CS disaccharides present or measured in a sample (i.e. sulfated CS disaccharides/sulfated+ unsulfated CS disaccharides). In some embodiments, “Charge CS” refers to the weighted sum of the concentration of all CS disaccharides divided by the total CS, where the weight is the count of sulfo groups in that disaccharide, i.e. 0 for 0s CS, 1 for 4s CS, 6s CS, and 2s CS, 2 for 2s6s CS, 4s6s CS, and 2s4s CS, and 3 for Tris CS (and this is the definition of “Charge CS” used in connection with the term “Charge CS” in the Example section herein).

As the measurement of “charge CS” is dependent on the measurement of other properties, i.e. the measurement of levels of sulfated and unsulfated CS disaccharides, this property is not referred to herein as an independent GAG property or CS property. Thus, up to 9 independent CS properties can be measured in the methods of the present invention, which are the 8 sulfated and unsulfated forms listed above, together with the total CS. In some embodiments all 9 of these independent CS properties are measured. In some embodiments only the 8 sulfated and unsulfated forms of CS disaccharides listed above are measured.

In some embodiments it is preferred to measure up to 8 (e.g. 1, 2, 3, 4, 5, 6, 7 or 8) or all 8 of the CS sulfation forms (i.e. the sulfated and unsulfated forms), together with total CS and charge CS. In preferred embodiments, at least one (or at least 2, 3, 4, 5, 6, 7 or 8) CS sulfation form is measured.

In some embodiments, one or more (or all) of the following GAG properties may be measured or determined: the relative level of 4s CS with respect to 6s CS (e.g. the ratio 4s CS/6s CS or the inverse ratio 6s CS/4s CS), the relative level of 6s CS with respect to 0s CS (e.g. the ratio 6s CS/0s CS or the inverse ratio 0s CS/6s CS) or the relative level of 4s CS with respect to 0s CS (e.g. the ratio 4s CS/0s CS or the inverse ratio 0s CS/4s CS). In some embodiments, one or more of the following GAG properties may be measured or determined: the relative level of 6s CS with respect to 0s CS (e.g. the ratio 6s CS/0s CS or the inverse ratio 0s CS/6s CS) or the relative level of 4s CS with respect to 0s CS (e.g. the ratio 4s CS/0s CS or the inverse ratio 0s CS/4s CS). In some embodiments, the relative level of 4s CS with respect to 6s CS (e.g. the ratio 4s CS/6s CS or the inverse ratio 6s CS/4s CS) is not measured or determined.

For example, for HS, there are 8 main sulfated and unsulfated forms (sulfation patterns, disaccharide sulfation forms) which are: 0s HS (also referred to as unsulfated HS), 2s HS (which is sulfated at the 2-position of GICA), Ns HS (which is sulfated at the N-position of the GlcNAc), 6s HS (which is sulfated at the 6-position of the GlcNAc), 2s6s HS (which is sulfated at the 2-position of GlcA and the 6-position of the GlcNAc), Ns6s HS (which is sulfated at the 6-position and N-position of GlcNAc), Ns2s HS (which is sulfated at the 2-position of GICA and the N-position of GlcNAc), Tris HS (which is sulfated at the 2-position of GICA and 6-position and N-position of GlcNAc, also referred to as trisulfated HS). Note that sulfation in position 3 of the GlcNAc is also possible but rarely observed.

Each of the above is a form of HS GAG (an HS GAG form or property) which may be measured or determined in the methods of the present invention. However, due to its rareity, in preferred embodiments of the invention, the sulfation form with sulfation in position 3 of the GlcNAc is not measured. Thus, in the methods of the invention, one or more (or all) of these 9 (or preferably 8) forms may be measured, for example up to 9 (or preferably up to 8), e.g. 1, 2, 3, 4, 5, 6, 7, 8 or all 9 of these sulfation forms may be measured. In some embodiments, measurement of all 8 of these sulfation forms (excluding the sulfation form with sulfation in position 3 of the GlcNAc) is preferred.

Another GAG property for HS which may be measured in the methods of the present invention is the total concentration of HS (also referred to herein as HS tot or Tot HS or Total HS) or the total level of HS. This is typically measured as a concentration, e.g. an absolute concentration, e.g. in μg/ml, as described elsewhere herein. In some embodiments, the total HS is measured by summing the level of all measured HS disaccharide forms listed above. In embodiments of the invention where the total concentration of HS is measured as one of the GAG properties, then it is preferred that at least one other GAG property or HS property is measured, e.g. a property that is not based on the total level of the other individual GAGs present (e.g. not total CS or total HA). In some embodiments the total concentration of HS is not measured. The measurement of one or more HS GAG properties is preferred in the methods of the invention, e.g. where the sample is blood or urine, in particular where the sample is a urine sample.

“Charge HS” is another GAG form or property which may be measured in the present invention, e.g. as part of the GAG profile. “Charge HS” refers to the total fraction of sulfated disaccharides of HS, i.e. the fraction of sulfated disaccharides of HS present or measured in a sample out of the total HS disaccharides present or measured in a sample (i.e. sulfated HS disaccharides/sulfated+ unsulfated HS disaccharides). In some embodiments, “Charge HS” refers to the weighted sum of the concentration of all HS disaccharides divided by the total HS, where the weight is the count of sulfo groups in that disaccharide, i.e. 0 for 0s HS, 1 for Ns HS, 6s HS, 2s HS, 2 for 2s6s HS, Ns6s HS, and Ns2s HS, and 3 for Tris HS (and this is the definition of “Charge HS” used in connection with the term “Charge HS” in the Example section herein).

As the measurement of “charge HS” is dependent on the measurement of other properties, i.e. the measurement of sulfated and unsulfated HS disaccharides, this property is not referred to herein as an independent GAG property or HS property. Thus, up to 10 independent HS properties can be measured in the methods of the present invention, which are the 9 sulfated and unsulfated forms listed above (preferably excluding the sulfation form with sulfation in position 3 of the GlcNAc), together with the total HS. Thus, in some embodiments 9 independent HS properties are measured (the 8 main sulfated and unsulfated HS forms plus total HS). In some embodiments only the 8 sulfated and unsulfated forms of HS disaccharides listed above are measured.

In some embodiments it is preferred to measure up to 8 (e.g. 1, 2, 3, 4, 5, 6, 7 or 8) or all 8 of the HS main sulfation forms (i.e. the sulfated and unsulfated forms listed above excluding the sulfation form with sulfation in position 3 of the GlcNAc), together with total HS and charge HS. In preferred embodiments, at least one (or at least 2, 3, 4, 5, 6, 7 or 8) HS sulfation form is measured.

In some embodiments 9 independent HS properties are measured (the 8 main sulfated and unsulfated HS forms plus total HS) and the 9 independent CS properties are measured, i.e. 18 independent GAG properties.

As described elsewhere herein, in some embodiments the level and/or chemical composition of hyaluronic acid (HA) may be additionally determined in a body fluid sample. Hyaluronic acid (HA) is typically non-sulfated. Accordingly, when HA is measured in accordance with the invention, it is typically and preferably the level (total level or total concentration) of HA that is measured (also referred to herein as HA tot or Tot HA or Total HA). This is typically measured as a concentration, e.g. in μg/ml, as described elsewhere herein. In some embodiments of the invention where the total concentration of HA is measured as one of the GAG properties, then at least one other GAG property (e.g. CS and/or HS property) is measured, e.g. a property that is not based on the total level of the other individual GAGs present (e.g. not total CS or total HS). In some embodiments, HA is not measured. In some embodiments, HA is not measured in urine. In some embodiments, HA is not measured in blood.

In some embodiments 9 independent HS properties are measured (the 8 main sulfated and unsulfated HS forms plus total HS) and the 9 independent CS properties are measured (the 8 main sulfated and unsulfated CS forms plus total CS) and total HA are measured, i.e. 19 independent GAG properties.

In some embodiments 8 independent HS properties are measured (the 8 main sulfated and unsulfated HS forms) and the 8 independent CS properties are measured (the 8 main sulfated and unsulfated CS forms) and total HA is measured, i.e. 17 independent GAG properties.

In some embodiments 8 independent HS properties are measured (the 8 main sulfated and unsulfated HS forms) and the 8 independent CS properties are measured (the 8 main sulfated and unsulfated CS forms), i.e. 16 independent GAG properties. CS (total CS) and HS (total HS) is typically measured in terms of an absolute concentration, e.g. in μg/ml, as described elsewhere herein.

The various CS sulfation forms and HS sulfation forms may also be measured in terms of an absolute concentration, e.g. in μg/ml. Thus, in some embodiments the level (or concentration) of a given CS sulfation form or a given HS sulfation form is an absolute level or absolute concentration of a given CS sulfation form or a given HS sulfation form.

However, the various CS sulfation forms and HS sulfation forms may alternatively, or additionally, be measured in terms of a relative concentration (or relative level). Thus, in some embodiments the level (or concentration) of a given CS sulfation forms or a given HS sulfation form is the relative level or relative concentration.

Thus the “level” or “concentration” of GAG sulfation forms may be its absolute concentration or its relative concentration. In some embodiments, both the absolute concentration and the relative concentration of one or more sulfated GAG forms is measured (or determined).

The “relative concentration” may be considered the mass fraction (e.g. in %) of a given CS sulfation form or a given HS sulfation form that is obtained (or calculated or determined) by normalizing its absolute concentration to the total concentration of the relevant GAG class, i.e. by normalizing its absolute concentration to the total CS concentration or total HS concentration (as appropriate).

Thus, the relative concentration of a given CS sulfation form may be considered the mass fraction (e.g. in %) of said given CS sulfation form that is obtained (or calculated or determined) by normalizing its absolute concentration by the total CS concentration.

The relative concentration of a given HS sulfation form may be considered the mass fraction (e.g. in %) of said given HS sulfation form that is obtained (or calculated or determined) by normalizing its absolute concentration by the total HS concentration. Relative concentration may be expressed in terms of a percentage (%).

Thus, in some embodiments the level (or concentration) of a given CS sulfation form or a given HS sulfation form may be the absolute concentration and/or the relative concentration of the given CS sulfation form or the given HS sulfation form. In some embodiments the level (or concentration) of a given CS sulfation form or a given HS sulfation form is the absolute concentration of the given CS sulfation form or the given HS sulfation form. In some embodiments the level (or concentration) of a given CS sulfation form or a given HS sulfation form is the relative concentration of the given CS sulfation form or the given HS sulfation form.

A relative concentration may be alternatively viewed as a “fraction” or “mass fraction” or “proportion” or “relative measurement” as discussed below.

As discussed above, GAG properties or GAG forms, e.g. disaccharide sulfation forms (with the exception of total CS or total HS) may be measured as a fraction size or fraction or mass fraction (e.g. μg/μg) or proportion or relative measurement, rather than as absolute levels or concentrations, for example are given a value of less than 1 or are normalised to 1 depending on the levels of all the sulfation forms for the relevant GAG class (or all the main sulfation forms for the relevant GAG class) measured in the sample (or are expressed in terms of a %). In other words, the level of each of the desired sulfation forms is measured independently and then normalised to 1. In other words, the level of each of the desired sulfation forms is measured independently and then its mass fraction or volume fraction or mole fraction is computed. These fractions may also be expressed as percentage. In other words, these fractions may also be normalised to 100. For example, in some embodiments, the fraction size of a given sulfated CS form or unsulfated CS form may be determined by measuring the level of the given sulfated CS form or unsulfated CS form and dividing this by the sum of the levels of all of the CS sulfation forms (or all of the main sulfation forms) and the unsulfated CS form measured (or present) in the sample. In some embodiments, the fraction size of a given sulfated HS form or unsulfated HS form may be determined by measuring the level of the given sulfated HS form or unsulfated HS form and dividing this by the sum of the levels of all of the HS sulfation forms (or main sulfation forms) and the unsulfated HS form measured (or present) in the sample. When calculating such fractions, it is preferred that at least the main sulfation forms of CS or HS are measured in order to be able to normalise the fraction of the particular individual sulfation form to 1.

In some embodiments, preferably at least the unsulfated forms of HS or CS are measured as the main sulfation form.

Relative measurements may be more easy to interpret, for example, a measurement of 0s CS of 0.6 indicates that 60% of the measured CS disaccharides are unsulfated. However, absolute levels can also be measured. Indeed, in some embodiments it is preferred to measure absolute concentrations of one or more sulfation forms.

In some preferred embodiments of the invention, the disaccharide composition (for example the specific sulfation patterns (e.g. sulfation forms)) of one or more (or all) of the disaccharides making up CS and/or HS is measured or determined. In more preferred embodiments one or more (or all) sulfation properties or forms of CS and/or HS such as those outlined above (e.g. 0s CS, 2s CS, etc), are measured or determined. Appropriate methods of doing this would be well known to a skilled person in the art and any of these could be used. However, a convenient method to achieve such quantification of disaccharide composition or the appropriate properties or forms of CS or HS (and separation of the disaccharide forms) is to use electrophoresis, in particular capillary electrophoresis, e.g. capillary electrophoresis with fluorescence detection, e.g. capillary electrophoresis with laser-induced fluorescence detection (CE-LIF). An alternative method is liquid chromatography, preferably HPLC (high-performance liquid chromatography), for example SAX HPLC. Preferably mass spectrometry is also used (e.g. HPLC-MS), for example electrospray ionization mass spectrometry (ESI-MS). Alternatively, mass spectrometry can be used without chromatography, e.g. liquid chromatography. One example is capillary electophoresis with laser-induced fluorescence detection. Another example is HPLC ESI-MS. Preferred methods involve high performance liquid chromatography (HPLC), preferably ultra-HPLC (UHPLC), in combination with mass spectrometry, e.g. MS/MS or triple quadropole mass spectrometry. Preferred methods comprise ultra-high-performance liquid chromatography (UHPLC) coupled with electrospray ionization triple-quadrupole mass spectrometry. Particularly preferred methods are outlined in the Examples.

In some methods of the invention where the levels of one or more individual disaccharide forms are measured, the GAGs are subjected to a processing step, for example a step of fragmentation or cleavage or digestion, e.g. by chemical digestion or enzyme treatment in order to obtain the disaccharide units which are then analysed. The enzyme may be a GAG lyase, e.g. a chondroitinase or a heparinase, or a combination of chondrotinases, or a combination of heparinases, or a combination of one or more chondroitinases and one or more heparinases. Preferably, the chondroitinase is Chondroitinase ABC or Chondroitinase B. Preferably the heparinase is Heparinase I-II-III. In preferred embodiments one or more chondrotinases and one or more heparinases are used, preferably Chondroitinase ABC and Heparinase I-II-III.

In some methods of the invention the GAGs in the sample are subjected to a step of extraction (e.g. using a proteolytic agent such as a protease, e.g. a non-specific protease, e.g. proteinase K) and/or purification, e.g. using an anion-exchange resin (or other means to purify GAGs based on the negative charge of the GAGs).

However, in preferred methods of the invention one or both of these steps is not carried out (i.e. there is no such extraction and/or no such purification). For example, in preferred embodiments of the invention, e.g. where the level and/or composition of the protein-free fraction of one or more GAGs is determined, then no such protein digestion (extraction) step is carried out. In other words, said methods do not involve a processing step in which samples are contacted with a proteolytic agent, such as for example a protease, e.g. proteinase K. As discussed elsewhere herein, omitting a processing step in which samples are contacted with a proteolytic agent means that the protein-free fraction of GAGs can be specifically analysed.

In other preferred embodiments of the invention, said methods do not involve a processing step in which the GAGs (e.g. one or both of the GAGs CS and HS) are purified from the sample based on the negative charge of said GAGs, e.g. using an anion-exchange resin. Without wishing to be bound by theory, omitting a processing step in which the GAGs (e.g. one or both of the GAGs CS and HS) are purified from the sample based on the negative charge of said GAGs (e.g. using an anion-exchange resin) simplifies the method and can lead to efficiencies in terms of the yield of GAGs obtained during processing of the body fluid sample.

In preferred methods of the present invention, methods do not involve a processing step in which samples are contacted with a proteolytic agent, such as for example a protease, e.g. proteinase K, and do not involve a processing step in which the GAGs (e.g. one or both of the GAGs CS and HS) are purified from the sample based on the negative charge of said GAGs, e.g. using an anion-exchange resin (or other means to purify GAGs based on the negative charge of the GAGs).

In some methods of the invention the GAGs in the sample (e.g. various different GAG forms in the sample) are subjected to a step of separation and/or quantification, as described elsewhere herein. For example, as discussed elsewhere herein, HPLC in combination with mass spectrometry may be used in preferred embodiments. Particularly preferred methods comprise ultra-high-performance liquid chromatography (UHPLC) coupled with electrospray ionization triple-quadrupole mass spectrometry.

Other methods which might be used are known in the art. However, examples are analytical techniques involving the use of antibodies to various GAG forms, e.g. techniques such as Western blot, ELISA or FACS, or methods involving agarose gel electrophoresis (e.g. fluorophore-assisted carbohydrate electrophoresis (FACE)) or polyacrylamide gel electrophoresis (PAGE).

Preferred methods of the invention provide a method of screening for cancer in a subject, said method comprising determining or measuring the amount or level and/or chemical composition, preferably in the protein-free fraction of GAGs, in a body fluid sample, wherein said determination comprises determining the level, preferably in said protein-free fraction, of one or more GAG properties selected from the group consisting of: one or more (or all) of the specific sulfated or unsulfated forms of CS or HS disaccharides, charge HS, charge CS, the total concentration of CS or the total concentration of HS.

Preferred methods of the invention provide a method of screening for cancer in a subject, said method comprising determining or measuring the amount or level and/or chemical composition, preferably in the protein-free fraction of GAGs, in a body fluid sample, wherein said determination comprises determining the level, preferably in said protein-free fraction, of one or more (or all) GAG properties selected from the group consisting of: one or more (or all) of the GAG properties selected from the group consisting of: the specific sulfated or unsulfated forms of CS or HS disaccharides selected from the group consisting of: 0s CS, 2s CS, 6s CS, 4s CS, 2s6s CS, 2s4s CS, 4s6s CS, Tris CS, 0s HS, 2s HS, 6s HS, 2s6s HS, Ns HS, Ns2s HS, Ns6s HS, Tris HS, the ratio of 4s CS to 6s CS, the ratio of 6s CS to 0s CS and the ratio of 4s CS to 0s CS; charge HS; charge CS; the total concentration of CS (CS tot); and the total concentration of HS (HS tot).

As mentioned above, the level (or concentration) of the specific sulfated or unsulfated forms of CS and HS may be an absolute concentration or a relative concentration.

Thus, in some embodiments GAG properties selected from the group consisting of 0s CS, 2s CS, 4s CS, 6s CS, 2s4s CS, 2s6s CS, 4s6s CS, Tris CS, charge CS, Total CS, 0s HS, 2s HS, Ns HS, 6s HS, 2s6s HS, Ns6s HS, Ns2s HS, Tris HS, HS sulfated at position 3 of the GlcNAc, Total HS, charge HS, can be measured or determined in methods of the invention. As described elsewhere herein, preferably HS sulfated at position 3 of the GlcNAc is not measured or determined. Thus, in some embodiments GAG properties selected from the group consisting of 0s CS, 2s CS, 4s CS, 6s CS, 2s4s CS, 2s6s CS, 4s6s CS, Tris CS, charge CS, Total CS, 0s HS, 2s HS, Ns HS, 6s HS, 2s6s HS, Ns6s HS, Ns2s HS, Tris HS, Total HS, charge HS can be measured or determined in methods of the invention. In some embodiments the level and/or chemical composition of hyaluronic acid (HA) may be additionally determined in a body fluid sample.

In some embodiments, for example where levels of various GAGs are determined in the protein-free fractions, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 6s CS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; the ratio of 4s CS to 0s CS; CS tot; HS tot; charge CS.

In some embodiments, for example where levels of various GAGs are determined in the protein-free fractions, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 6s CS, absolute concentration of 2s6s CS, relative concentration of 4s CS, relative concentration of 2s6s CS, relative concentration of 6s CS; CS tot; HS tot; charge CS.

In some embodiments, for example where levels of various GAGs are determined in the protein-free fractions, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS, absolute concentration of 6s CS; the ratio of 6s CS to 0s CS; the ratio of 4s CS to 0s CS; CS tot; HS tot; charge CS.

In some embodiments, for example where levels of various GAGs are determined in the protein-free fractions, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; the ratio of 4s CS to 0s CS; CS tot; HS tot; charge CS.

In some embodiments, for example where levels of various GAGs are determined in the protein-free fractions, and wherein said body fluid sample is urine, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 6s CS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; CS tot; HS tot; charge CS.

In some embodiments, for example where levels of various GAGs are determined in the protein-free fractions, and wherein said body fluid sample is urine, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, absolute concentration of 0s Cs, relative concentration of 0s CS, absolute concentration of 0s HS, absolute concentration of Ns HS, absolute concentration of 6s CS, absolute concentration of 2s6s CS, relative concentration of 2s6s CS, relative concentration of 6s CS; CS tot; HS tot; charge CS.

In some embodiments, for example where levels of various GAGs are determined in the protein-free fractions, and wherein said body fluid sample is urine, the methods of the invention comprise measurement or determination of one or more (or all) of the GAG properties selected from the group consisting of: the specific sulfated or unsulfated forms of CS or HS disaccharides selected from the group consisting of: absolute concentration of 4s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; CS tot; HS tot.

In some embodiments, for example where levels of various GAGs are determined in the protein-free fractions, and wherein said body fluid sample is urine, the methods of the invention comprise measurement or determination of one or more (or all) of the GAG properties selected from the group consisting of: the specific sulfated or unsulfated forms of CS or HS disaccharides selected from the group consisting of: absolute concentration of 0s HS, relative concentration of 6s CS, relative concentration of Ns HS, relative concentration of 0s HS, relative concentration of 2s6s CS, relative concentration of 4s CS, relative concentration of 0s CS, absolute concentration of 4s CS, absolute concentration of 6s CS, absolute concentration of 0s CS; the ratio of 6s CS to 0s CS; CS tot; HS tot.

In some embodiments, for example where levels of various GAGs are determined in the protein-free fractions, and wherein said body fluid sample is blood, e.g. plasma, the methods of the invention comprise measurement or determination of one or more (or all) of the GAG properties selected from the group consisting of: the specific sulfated or unsulfated forms of CS or HS disaccharides selected from the group consisting of: absolute concentration of 0s CS, relative concentration of 0s CS, absolute concentration of 4s CS, relative concentration of 4s CS; the ratio of 4s CS to 0s CS; CS Tot; charge CS.

In some embodiments, for example where levels of various GAGs are determined in the protein-free fractions, and wherein said body fluid sample is blood, e.g. plasma, the methods of the invention comprise measurement or determination of one or more (or all) of the GAG properties selected from the group consisting of: the specific sulfated or unsulfated forms of CS or HS disaccharides selected from the group consisting of: absolute concentration of 0s CS, relative concentration of 0s CS, absolute concentration of 4s CS, relative concentration of 4s CS; CS Tot; charge CS.

In some embodiments, for example where levels of various GAGs are determined in the protein-free fractions, and wherein said body fluid sample is blood, e.g. plasma, the methods of the invention comprise measurement or determination of one or more (or all) of the GAG properties selected from the group consisting of: the specific sulfated or unsulfated forms of CS or HS disaccharides selected from the group consisting of: absolute concentration of 0s CS, relative concentration of 4s CS; the ratio of 4s CS to 0s CS.

In some embodiments, for example where levels of various GAGs are determined in the protein-free fractions, GAG properties can be determined from both blood, e.g. plasma, and urine. Preferred and exemplary GAG properties are as outlined in the paragraphs above, and any combination of these blood and urine properties can be used. Such determinations, involving both blood, e.g. plasma, and urine samples, can be referred to as combined determinations, and for example can give rise to combined scores.

In some embodiments, for example where levels of various GAGs are determined in the protein-free fractions, and wherein both blood, e.g. plasma, and urine body fluid samples are used, the methods of the invention comprise
measurement or determination in said urine sample of one or more (or all) of the GAG properties selected from the group consisting of: relative concentration of 0s CS, absolute concentration of 0s HS, absolute concentration of Ns HS, relative concentration of 2s6s CS, relative concentration of 0s HS, relative concentration of Ns HS; HS tot; and charge CS; and
measurement or determination in said blood, e.g. plasma, sample of one or more (or all) of the GAG properties selected from the group consisting of: absolute concentration of 0s CS; relative concentration of 4s CS; and CS tot.

In some embodiments, for example where levels of various GAGs are determined in the protein-free fractions, and wherein both blood, e.g. plasma, and urine body fluid samples are used, the methods of the invention comprise
measurement or determination in said urine sample of one or more (or all) of the GAG properties selected from the group consisting of: absolute concentration of 0s HS, relative concentration of Ns HS, relative concentration of 0s CS, relative concentration of 0s HS, absolute concentration of 4s CS, absolute concentration of 0s CS, relative concentration of 4s CS, relative concentration of 6s CS, absolute concentration of 6s CS; the ratio of 6s CS to 0s CS; CS tot; and charge CS; and
measurement or determination in said blood, e.g. plasma, sample of one or both of the GAG properties selected from the group consisting of: the ratio of 4s CS to 0s CS; and CS tot.

In some embodiments, for example where levels of various GAGs are determined in the protein-free fractions, the methods of the invention comprise measurement or determination in a urine sample of one or more (or all) of the GAG properties selected from the group consisting of: absolute concentration of 0s HS, absolute concentration of Ns HS, absolute concentration of 4s CS, absolute concentration of 6s CS. In some such embodiments, an increase in a urine sample of one or both of: absolute concentration of 0s HS or absolute concentration of 4s CS, for example in comparison to a control level, is indicative of cancer in said subject. In some such embodiments, a decrease in a urine sample of one or both of: absolute concentration of Ns HS or absolute concentration of 6s CS, for example in comparison to a control level, is indicative of cancer in said subject.

In some embodiments, for example where levels of various GAGs are determined in the protein-free fractions, the methods of the invention comprise measurement or determination in a blood (e.g. plasma) sample of: absolute concentration of 0s CS. In some embodiments, in methods of screening for cancer an increase in a blood (e.g. plasma) sample of the absolute concentration of 0s CS, for example in comparison to a control level, is indicative of cancer in said subject.

In some embodiments, for example where levels of various GAGs are determined in the protein-free fractions, and wherein both blood, e.g. plasma, and urine body fluid samples are used, the methods of the invention comprise measurement or determination in said urine sample of one or more (or all) of the GAG properties selected from the group consisting of: absolute concentration of 0s HS, absolute concentration of Ns HS, absolute concentration of 4s CS, absolute concentration of 6s CS; and measurement or determination in said blood, e.g. plasma, sample of: absolute concentration of 0s CS. In some such embodiments, an increase in a urine sample of one or both of: absolute concentration of 0s HS or absolute concentration of 4s CS, for example in comparison to a control level, is indicative of cancer in said subject. In some such embodiments, a decrease in a urine sample of one or both of: absolute concentration of Ns HS or absolute concentration of 6s CS, for example in comparison to a control level, is indicative of cancer in said subject. In some embodiments, in methods of screening for cancer an increase in a blood (e.g. plasma) sample of the absolute concentration of 0s CS, for example in comparison to a control level, is indicative of cancer in said subject.

In some embodiments, for example where levels of various GAGs are determined in the protein-free fractions, preferred methods of the invention comprise measurement or determination of the level of one or both of the GAG properties selected from the group consisting of: the absolute concentration of 0s CS and CS Tot.

In some embodiments, for example where levels of various GAGs are determined in the protein-free fractions, an increase in the level of one or more (or all) of the GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 6s CS, absolute concentration of 2s6s CS, relative concentration of 4s CS, relative concentration of 2s6s CS; CS tot; HS tot, e.g. in comparison to a control level, is indicative of cancer (preferred cancers can be derived from elsewhere herein e.g. Table A).

In some embodiments, for example where levels of various GAGs are determined in the protein-free fractions, a decrease in the level of one or more (or all) of the GAG properties selected from the group consisting of: Charge CS and relative concentration of 6s CS, e.g. in comparison to a control level, is be indicative of cancer (preferred cancers can be derived from elsewhere herein e.g. Table A).

In some embodiments, for example where levels of various GAGs are determined in the protein-free fractions, wherein said body fluid sample is blood, e.g. plasma, an increase in the level of one or more (or all) of the GAG properties selected from the group consisting of: the specific sulfated or unsulfated forms of CS or HS disaccharides selected from the group consisting of: absolute concentration of 0s CS, relative concentration of 0s CS, absolute concentration of 4s CS, relative concentration of 4s CS; CS Tot, e.g. in comparison to a control level, is indicative of cancer (preferred cancers can be derived from elsewhere herein e.g. Table A).

In some embodiments, for example where levels of various GAGs are determined in the protein-free fractions, wherein said body fluid sample is blood, e.g. plasma, a decrease in Charge CS, e.g. in comparison to a control level, is indicative of cancer (preferred cancers can be derived from elsewhere herein e.g. Table A).

In some embodiments, for example where levels of various GAGs are determined in the protein-free fractions, wherein said body fluid sample is urine, an increase in the level of one or more (or all) of the GAG properties selected from the group consisting of: the specific sulfated or unsulfated forms of CS or HS disaccharides selected from the group consisting of: absolute concentration of 4s CS, absolute concentration of 0s Cs, relative concentration of 0s CS, absolute concentration of 0s HS, absolute concentration of Ns HS, absolute concentration of 6s CS, absolute concentration of 2s6s CS, relative concentration of 2s6s CS; CS tot; HS tot, e.g. in comparison to a control level, is indicative of cancer (preferred cancers can be derived from elsewhere herein e.g. Table A).

In some embodiments, for example where levels of various GAGs are determined in the protein-free fractions, wherein said body fluid sample is urine, a decreased in the level of one or both of the GAG properties Charge CS or the relative concentration of 6s CS, e.g. in comparison to a control level, is indicative of cancer (preferred cancers can be derived from elsewhere herein e.g. Table A).

Although the methods of the invention can involve the determination of the level of only one of the listed GAG properties, preferably said methods comprise determining the level of more than one of said GAG properties, more preferably said methods comprise determining the level of two or more, three or more, four or more, or all, of said GAG properties. In some embodiments said methods comprise determining the level of up to 8, or all 8, of the sulfated and unsulfated CS forms: 0s CS, 2s CS, 6s CS, 4s CS, 2s6s CS, 2s4s CS, 4s6s CS and Tris CS, optionally together with total CS and/or charge CS; and/or determining the level of up to 8, or all 8, of the sulfated and unsulfated HS forms: 0s HS, 2s HS, 6s HS, 2s6s HS, Ns HS, Ns2s HS, Ns6s HS and Tris HS, optionally together with total HS and/or charge HS.

In some embodiments, particularly preferred GAG forms to be measured or determined in the methods of the invention are one or more (or all) of: absolute concentration of 0s CS, CS Tot, absolute concentration of 4s CS, and absolute concentration of 0s HS. In some such embodiments, an alteration, preferably an increase, in the level of one or more (or all) of said GAG forms, e.g. in comparison to a control level, is indicative of cancer.

In some embodiments, particularly preferred GAG forms to be measured or determined in the methods of the invention are one or both of absolute concentration of 0s CS and CS Tot. In some such embodiments, an alteration, preferably an increase, in the level of one or both of said GAG forms, e.g. in comparison to a control level, is indicative of cancer. Typically and preferably, the level of said one or both GAG forms is the level in blood (e.g. plasma) or urine. Thus, in some embodiments the level of one or both of said GAG forms is the level in a blood (e.g. plasma) sample. Thus, in some embodiments the level of one or both of said GAG forms is the level in a urine sample.

In some embodiments in which one or both of absolute concentration of 0s CS and CS Tot are determined, preferably the cancer is selected from the group consisting of uterine cancer (e.g. cervix squamous cell carcinoma or endometrial cancer), blood cancer (e.g. a lymphoma such as chronic lymphoid leukaemia), brain tumor (e.g. diffuse glioma), a gastro-intestinal endocrine tumour, lung cancer (e.g. non-small cell lung cancer), ovarian cancer, and kidney cancer (e.g. renal cell cancer).

In some embodiments in which one or both of absolute concentration of 0s CS and CS Tot are determined and in which the body fluid sample is blood (e.g. plasma), preferably the cancer is selected from the group consisting of: uterine cancer (e.g. cervix squamous cell carcinoma or endometrial cancer), blood cancer (e.g. a lymphoma such as chronic lymphoid leukaemia), brain tumor (e.g. diffuse glioma), a gastro-intestinal endocrine tumour, head and neck cancer (e.g. head and neck squamous cell carcinoma), lung cancer (e.g. non-small cell lung cancer), ovarian cancer, and kidney cancer (e.g. renal cell cancer).

In some embodiments in which one or both of absolute concentration of 0s CS and CS Tot are determined and in which the body fluid sample is urine, preferably the cancer is selected from the group consisting of: lung cancer (e.g. non-small cell lung cancer), bladder cancer, and kidney cancer (e.g. renal cell cancer).

In preferred embodiments an increase in the level of one or both of absolute concentration of 0s CS and CS Tot, e.g. in comparison to a control level, is indicative of cancer (e.g. one or more of said cancer types mentioned in the paragraphs above in connection with embodiments in which one or both of absolute concentration of 0s CS and CS Tot are determined).

In some embodiments, a particularly preferred GAG form to be measured or determined in the methods of the invention is the absolute concentration of 0s CS. In some such embodiments, an alteration, preferably an increase, in the level of said GAG form, e.g. in comparison to a control level, is indicative of cancer. Typically and preferably, the level of said GAG form is the level in blood (e.g. plasma) or urine. Thus, in some embodiments the level of said GAG form is the level in a blood (e.g. plasma) sample. Thus, in some embodiments the level said GAG form is the level in a urine sample.

In some embodiments in which the absolute concentration of 0s CS is determined, preferably the cancer is selected from the group consisting of: breast cancer, colorectal cancer, uterine cancer (e.g. cervix squamous cell carcinoma or endometrial cancer), blood cancer (e.g. a lymphoma such as chronic lymphoid leukaemia or diffuse large B-cell lymphoma), brain tumor (e.g. diffuse glioma), a gastro-intestinal endocrine tumour, lung cancer (e.g. non-small cell lung cancer), ovarian cancer, and kidney cancer (e.g. renal cell cancer).

In some embodiments in which the absolute concentration of 0s CS is determined and in which the body fluid sample is blood (e.g. plasma), preferably the cancer is selected from the group consisting of: breast cancer, colorectal cancer, uterine cancer (e.g. cervix squamous cell carcinoma or endometrial cancer), blood cancer (e.g. a lymphoma such as chronic lymphoid leukaemia or diffuse large B-cell lymphoma), brain tumor (e.g. diffuse glioma), a gastro-intestinal endocrine tumour, head and neck cancer (e.g. head and neck squamous cell carcinoma), lung cancer (e.g. non-small cell lung cancer), ovarian cancer, and kidney cancer (e.g. renal cell cancer).

In some embodiments in which the absolute concentration of 0s CS is determined and in which the body fluid sample is urine, preferably the cancer is selected from the group consisting of: lung cancer (e.g. non-small cell lung cancer), bladder cancer, and kidney cancer (e.g. renal cell cancer).

In preferred embodiments an increase in the absolute concentration of 0s CS, e.g. in comparison to a control level, is indicative of cancer (e.g. one or more of said cancer types mentioned in the paragraphs above in connection with embodiments in which absolute concentration of 0s CS is determined).

In some embodiments, a particularly preferred GAG form to be measured or determined in the methods of the invention is CS Tot. In some such embodiments, an alteration, preferably an increase, in the level of said GAG form, e.g. in comparison to a control level, is indicative of cancer. Typically and preferably, the level of said GAG form is the level in blood (e.g. plasma) or urine. Thus, in some embodiments the level of said GAG form is the level in a blood (e.g. plasma) sample. Thus, in some embodiments the level said GAG form is the level in a urine sample.

In some embodiments in which CS Tot is determined, preferably the cancer is selected from the group consisting of: uterine cancer (e.g. cervix squamous cell carcinoma or endometrial cancer), blood cancer (e.g. a lymphoma such as chronic lymphoid leukaemia), brain tumor (e.g. diffuse glioma), a gastro-intestinal endocrine tumour, head and neck cancer (e.g. head and neck squamous cell carcinoma), lung cancer (e.g. non-small cell lung cancer), ovarian cancer, bladder cancer, and kidney cancer (e.g. renal cell cancer).

In some embodiments in which CS Tot is determined and in which the body fluid sample is blood (e.g. plasma), preferably the cancer is selected from the group consisting of: uterine cancer (e.g. cervix squamous cell carcinoma or endometrial cancer), blood cancer (e.g. a lymphoma such as chronic lymphoid leukaemia), brain tumor (e.g. diffuse glioma), a gastro-intestinal endocrine tumour, head and neck cancer (e.g. head and neck squamous cell carcinoma), lung cancer (e.g. non-small cell lung cancer), ovarian cancer, bladder cancer, prostate cancer, and kidney cancer (e.g. renal cell cancer).

In some embodiments in which CS Tot is determined and in which the body fluid sample is urine, preferably the cancer is selected from the group consisting of: head and neck cancer (e.g. head and neck squamous cell carcinoma), lung cancer (e.g. non-small cell lung cancer), bladder cancer, and kidney cancer (e.g. renal cell cancer).

In preferred embodiments an increase in CS Tot, e.g. in comparison to a control level, is indicative of cancer (e.g. one or more of said cancer types mentioned in the paragraphs above in connection with embodiments in which CS Tot is determined).

In some embodiments, a particularly preferred GAG form to be measured or determined in the methods of the invention is the absolute concentration of 4s CS. In some such embodiments, an alteration, preferably an increase, in the level of said GAG form, e.g. in comparison to a control level, is indicative of cancer. Typically and preferably, the level of said GAG form is the level in blood (e.g. plasma) or urine. Thus, in some embodiments the level of said GAG form is the level in a blood (e.g. plasma) sample. Thus, in some embodiments the level said GAG form is the level in a urine sample.

In some embodiments in which the absolute concentration of 4s CS is determined, preferably the cancer is a genitourinary cancer or a respiratory tract cancer.

In some embodiments in which the absolute concentration of 4s CS is determined, preferably the cancer is selected from the group consisting of: head and neck cancer (e.g. head and neck squamous cell carcinoma), lung cancer (e.g. non-small cell lung cancer) and bladder cancer.

In some embodiments in which the absolute concentration of 4s CS is determined and in which the body fluid sample is blood (e.g. plasma), preferably the cancer is a genitourinary cancer or a respiratory tract cancer.

In some embodiments in which the absolute concentration of 4s CS is determined and in which the body fluid sample is blood (e.g. plasma), preferably the cancer is selected from the group consisting of: head and neck cancer (e.g. head and neck squamous cell carcinoma), lung cancer (e.g. non-small cell lung cancer), bladder cancer, prostate cancer, and kidney cancer (e.g. renal cell cancer).

In some embodiments in which the absolute concentration of 4s CS is determined and in which the body fluid sample is urine, preferably the cancer is selected from the group consisting of: head and neck cancer (e.g. head and neck squamous cell carcinoma), lung cancer (e.g. non-small cell lung cancer) and bladder cancer.

In preferred embodiments an increase in the absolute concentration of 4s CS, e.g. in comparison to a control level, is indicative of cancer (e.g. one or more of said cancer types mentioned in the paragraphs above in connection with embodiments in which absolute concentration of 4s CS is determined).

In some embodiments, a particularly preferred GAG form to be measured or determined in the methods of the invention is the absolute concentration of 0s HS. In some such embodiments, an alteration, preferably an increase, in the level of said GAG form, e.g. in comparison to a control level, is indicative of cancer.

In some embodiments in which the absolute concentration of 0s HS is determined in which the body fluid sample is urine, preferably the cancer is selected from the group consisting of: head and neck cancer (e.g. head and neck squamous cell carcinoma), lung cancer (e.g. non-small cell lung cancer), and kidney cancer (e.g. renal cell cancer).

In preferred embodiments an increase in the absolute concentration of 0s HS, e.g. in comparison to a control level, is indicative of cancer (e.g. one or more of said cancer types mentioned in the paragraphs above in connection with embodiments in which 0s HS is determined).

In some preferred embodiments, particularly preferred GAG forms to be measured or determined in the methods of the invention are unsulfated GAG forms, 0s CS and/or 0s HS, typically the absolute concentration of said GAG forms. Preferably an increase in the level of one or both of said GAG forms, e.g. in comparison to a control level, is indicative of cancer (e.g. a cancer as set out elsewhere herein. Typically and preferably, the level of said unsulfated GAG forms is the level in blood (e.g. plasma) or urine. Thus, in some embodiments the level of said unsulfated GAG forms is the level in a blood (e.g. plasma) sample. Thus, in some embodiments the level said unsulfated GAG forms is the level in a urine sample.

In some embodiments, particularly preferred GAG forms to be measured or determined in the methods of the invention are one or more (or all) of absolute concentration of 4s CS, absolute concentration of 0s CS and CS Tot (e.g. in blood, e.g. plasma, samples). In some embodiments, particularly preferred GAG forms to be measured or determined in the methods of the invention are one or more (or all) of absolute concentration of 4s CS and absolute concentration of 0s CS (e.g. in blood, e.g. plasma, samples). In some such embodiments, an alteration, preferably an increase, in the level of one or more (or all) of said GAG forms, e.g. in comparison to a control level, is indicative of cancer (e.g. one or more of said cancer types mentioned in the paragraphs above in connection with embodiments in which absolute concentration of 4s CS or absolute concentration of 0s CS or CS Tot is determined).

In some embodiments, GAG forms to be measured or determined in method of the invention are one or more of the GAG forms set forth in Table A herein. In some embodiments, an increase (preferably in comparison to a control level) in the level of one or more of the GAG forms reported in Table A herein as being increased in cancer samples as compared to healthy samples is indicative of cancer. In some embodiments, a decrease (preferably in comparison to a control level) in the level of one or more of the GAG forms reported in Table A herein as being decreased in cancer samples as compared to healthy samples is indicative of cancer.

In some embodiments, a particularly preferred GAG form to be measured or determined in the methods of the invention is the absolute concentration of 4s CS. In some such embodiments, an alteration, preferably an increase, in the level of said GAG form, e.g. in comparison to a control level, is indicative of cancer.

In some embodiments in which the level of 0s CS is determined (or in which the screening e.g. diagnosis is based on an altered level of 0s CS) it may be preferred that the cancer is not kidney cancer (e.g. renal cell cancer) and/or is not prostate cancer.

In some embodiments in which the level of 0s CS is determined (or in which the screening e.g. diagnosis is based on an altered level of 0s CS) and in which the body fluid is urine, it may be preferred that the cancer is not kidney cancer (e.g. renal cell cancer) and/or is not prostate cancer.

In some embodiments in which the level of 0s CS is determined (or in which the screening e.g. diagnosis is based on an altered level of 0s CS) and in which the body fluid is plasma, it may be preferred that the cancer is not prostate cancer.

In some embodiments in which the level of 4s CS is determined (or in which the screening e.g. diagnosis is based on an altered level of 4s CS) and in which the body fluid is plasma, it may be preferred that the cancer is not kidney cancer (e.g. renal cell cancer) and/or is not skin cancer (e.g. melanoma).

In any embodiments which refer to the determination of the level of one or more from a certain list of GAG forms (or GAG properties), in some such embodiments the level of all the listed GAG forms (or GAG properties) may be determined.

In some embodiments, the level of a single GAG form (GAG property) is determined.

In one embodiment of methods of screening for cancer, the method comprises determining the level in a sample of one or more GAG features (GAG properties) that are identified in Table A herein as being significantly altered between cancer and healthy samples, i.e. those features with a “% in ROPE” (Region of Practical Equivalence) of less than 5.00 (which corresponds to a value of less than 0.05 in the “ROPE_Percentage” column of Table A, preferably the value in the “ROPE_Percentage” column of Table A may be less than 0.04. 0.03, 0.02, 0.01 or even a value of 0.00).

In other embodiments of the present invention, the level of more than one of the GAG forms (GAG properties) is determined (e.g. the level of two or more GAG forms, or three or more GAG forms, or four or more GAG forms, or five or more GAG forms is determined). By “more than one” is meant 2, 3, 4, 5, 6, 7, 8, 9, 10, 11, 12, 13, etc. In any list of markers or GAG properties provided herein, it is a preferred embodiment that all are measured. Also, a determination of the level of each and every possible combination of the GAG forms can be performed.

Thus, in some embodiments multi-marker methods are performed. Determining the level of multiple of the GAG forms (biomarker multiplexing) may improve screening (e.g. diagnostic) accuracy.

Thus, although markers (GAG forms) can be used in the methods of the invention individually, they can also be used in combination, e.g. in the form of a multi-marker assay.

In some embodiments that comprise determining the level in a sample of more than one GAG feature (GAG property), each of said more than one GAG features may be identified in Table A herein as being significantly altered between cancer and healthy samples, i.e. those features with a “% in ROPE” (Region of Practical Equivalence) of less than 5.00 (which corresponds to a value of less than 0.05 in the “ROPE_Percentage” column of Table A, preferably the value in the “ROPE_Percentage” column of Table A may be less than 0.04. 0.03, 0.02, 0.01 or even a value of 0.00).

In some embodiments, the level of a single GAG form (GAG property) is used for the basis of the screening for cancer, e.g. a diagnosis, prognosis may be made on the basis of the level of a single GAG form in some embodiments. In other embodiments, more than one (e.g. 2, 3, 4, 5, 6, 7, 8, 9, 10, 11, 12, 13, etc.) GAG form is used for the basis of the screening for cancer, e.g. a diagnosis, prognosis may be made on the basis of the level of more than one GAG form in some embodiments, e.g. on the basis of any one of the groups (or sub-groups) of GAG forms set out herein. In some embodiments, where one GAG form or a group (or sub-group or subset) of GAG forms is used for the basis of the screening for cancer (e.g. diagnosis or prognosis), the level of one or more (or all) of the other GAG forms (or GAG properties) described herein may be additionally determined or measured.

Based on the observed alterations in the levels of various GAG forms in cancer patients versus healthy patients, if desired, scoring methods, scoring systems, markers or formulas can be designed which use such levels of various GAG forms in order to arrive at an indication, e.g. in the form of a value or score, which can then be used for screening (e.g. diagnosis, etc.). Appropriate scoring systems and parameters (e.g. GAG forms) to be measured can readily be designed based for example on the data described herein, for example the data in Table A, for example, based on one or more of the individual GAG features or properties which show significant differences in particular samples (blood (e.g. plasma) or urine) as indicated in Table A. In particular, GAG properties in Table A that are most different between cancer samples and healthy samples (e.g. preferably one or more or all properties which have a % in ROPE value of 0.00 or close to 0.00, e.g. one or more or all properties which have a % in ROPE value equal to or less than 5.00 or 4.00 or 3.00 or 2.00 or 1.00) may be selected. A % in ROPE value equal to or less than 5.00 or 4.00 or 3.00 or 2.00 or 1.00 corresponds to a value of equal to or less than 0.05, equal to or less than 0.04, equal to or less than 0.03, equal to or less than 0.02, equal to or less than 0.01 in the “ROPE_Percentage” column of Table A.

In some embodiments in accordance with the present invention, the chemical composition may be expressed in terms of score (or GAG score), said score being based on the measured level of one or more (preferably more than one) or all of the GAG properties (or groups of GAG properties) described herein.

In some embodiments, a score (or GAG score) may be based on (or derived or calculated or computed using) one or more (or all) measured (or determined) GAG properties selected from the group consisting of: the specific sulfated or unsulfated forms of CS or HS disaccharides selected from the group consisting of: absolute concentration of 4s CS, absolute concentration of 0s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS, absolute concentration of 6s CS; the ratio of 6s CS to 0s CS; the ratio of 4s CS to 0s CS; CS tot; HS tot; charge CS.

In some embodiments, a score (or GAG score) may be based on (or derived or calculated or computed using) one or more (or all) measured (or determined) GAG properties selected from the group consisting of: absolute concentration of 4s CS, absolute concentration of 0s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; the ratio of 4s CS to 0s CS; CS tot; HS tot; charge CS.

In some embodiments, when the body fluid sample is a blood (e.g. plasma) sample, a score (or GAG score or blood GAG score or plasma GAG score) may be based on (or derived or calculated or computed using) one or more (or all) measured (or determined) GAG properties selected from the group consisting of: absolute concentration of 0s CS, relative concentration of 4s CS; the ratio of 4s CS to 0s CS.

In some embodiments, when the body fluid sample is a urine sample, a score (or GAG score or urine GAG score) may be based on (or derived or calculated or computed using) one or more (or all) measured (or determined) GAG properties selected from the group consisting of: absolute concentration of 4s CS, relative concentration of 0s CS, absolute concentration of 0s HS, absolute concentration of Ns HS, absolute concentration of 2s6s CS, relative concentration of 4s CS, relative concentration of 2s6s CS, relative concentration of 6s CS, relative concentration of 0s HS, relative concentration of Ns HS; the ratio of 6s CS to 0s CS; CS tot; HS tot.

In some embodiments, when the body fluid sample is a urine sample, a score (or GAG score or urine GAG score) may be based on (or derived or calculated or computed using) one or more (or all) measured (or determined) GAG properties selected from the group consisting of: the specific sulfated or unsulfated forms of CS or HS disaccharides selected from the group consisting of: absolute concentration of 0s HS, relative concentration of 6s CS, relative concentration of Ns HS, relative concentration of 0s HS, relative concentration of 2s6s CS, relative concentration of 4s CS, relative concentration of 0s CS, absolute concentration of 4s CS, absolute concentration of 6s CS, absolute concentration of 0s CS; the ratio of 6s CS to 0s CS; CS tot; HS tot.

In some embodiments, a score may be based on (or derived or calculated or computed using) measured levels of one or more GAG properties in more than one type of body fluid sample (e.g. blood (e.g. plasma) and urine). In some such embodiments, the GAG properties measured in each type of body fluid may be the same or they may be different.

In some embodiments, a score (or GAG score or combined GAG score) may be based on (or derived or calculated or computed using) (i) one or more (or all) measured (or determined) GAG properties in a blood (e.g. plasma) sample selected from the group consisting of: absolute concentration of 0s CS; relative concentration of 4s CS; and CS tot; and (ii) one or more (or all) measured (or determined) GAG properties in a urine sample selected from the group consisting of: relative concentration of 0s CS, absolute concentration of 0s HS, absolute concentration of Ns HS, relative concentration of 2s6s CS, relative concentration of 0s HS, relative concentration of Ns HS; HS tot; and charge CS.

In some embodiments, a score (or GAG score or combined GAG score) may be based on (or derived or calculated or computed using) (i) one or both measured (or determined) GAG properties in a blood (e.g. plasma) sample selected from the group consisting of: the ratio of 4s CS to 0s CS; and CS tot; and (ii) one or more (or all) measured (or determined) GAG properties in a urine sample selected from the group consisting of: absolute concentration of 0s HS, relative concentration of Ns HS, relative concentration of 0s CS, relative concentration of 0s HS, absolute concentration of 4s CS, absolute concentration of 0s CS, relative concentration of 4s CS, relative concentration of 6s CS, absolute concentration of 6s CS; the ratio of 6s CS to 0s CS; CS tot; and charge CS.

In some embodiments, a score (or GAG score or combined GAG score) may be based on (or derived or calculated or computed using) (i) the measured (or determined) absolute concentration of 0s CS in a blood (e.g. plasma) sample; and (ii) one or more (or all) measured (or determined) GAG properties in a urine sample selected from the group consisting of: absolute concentration of 0s HS, absolute concentration of Ns HS, absolute concentration of 4s CS, absolute concentration of 6s CS.

A score based on one or more GAG properties measured in a urine sample may be referred to as a urine score (or a urine GAG score). A score based on one or more GAG properties measured in a blood (e.g. plasma) sample may be referred to as a blood score or plasma score, as appropriate (or a blood GAG score or a plasma GAG score). A score based on one or more GAG properties measured in more than one type of body fluid sample (e.g. blood (e.g. plasma) and urine) may be referred to as a combined score (or a combined GAG score).

In some embodiments, a preferred blood (e.g. plasma) score (or blood GAG score or plasma GAG score) may be based on (or derived or calculated or computed using) the measured (or determined) GAG properties described in connection with the plasma score described in the Example section herein. In some embodiments, a preferred urine score (or urine GAG score) may be based on (or derived or calculated or computed using) the measured (or determined) GAG properties described in connection with the urine scores described in the Example section herein. In some embodiments, a preferred combined score (or combined GAG score) may be based on (or derived or calculated or computed using) the measured (or determined) GAG properties described in connection with the combined scores described in the Example section herein.

In some embodiments, appropriate threshold or cut-off values (e.g. used to declare a sample positive or negative) for use with scores can be designed by a person skilled in the art. By way of example, a cut-off (or threshold) value may be calculated based on ROC curves. In embodiments in which the aim is to categorise a subject as having a high risk cancer (e.g. having poor or poorer overall survival prospects) or having a low risk cancer (e.g. having good or better overall survival prospects) one example of a cut-off (or threshold) value may be the median score across a population of cancer subjects or across a plurality of cancer samples. Alternatively, maximally selected rank statistics may be used to identify a cut-off value (or cut-off score).

In some embodiments, screening (e.g. diagnosis, etc.) may be done by comparing a given score for a sample (or subject) to be diagnosed with a threshold or cut-off value, and assigning a result (e.g. positive or negative diagnosis etc.) based on whether the score determined is above or below (e.g. significantly above or significantly below) a cut-off value.

In some cases, a high score gives rise to a positive screening for, diagnosis of etc., the presence of cancer. Where the aim is to provide a scoring system in which a high score is indicative of the presence of cancer then conveniently a scoring system can be designed as a ratio or fraction, where the numerator is the sum of the values associated with one or more GAG properties (GAG forms) associated with cancer and the denominator is the sum of the values associated with one or more GAG properties (GAG forms) associated with the healthy state.

Of course, alternative scoring systems (or formulae) could equally be designed where for example the numerator is the sum of the values associated with one or more GAG properties (GAG forms) associated with the healthy state and the denominator is the sum of the values associated with one or more GAG properties (GAG forms) associated with cancer, and a low score is indicative of the presence of cancer.

Any scoring methods, scoring systems, markers or formulas can be used that comprise any appropriate combination of the GAG properties in order to arrive at an indication, e.g. in the form of a value or score, which can then be used for screening (e.g. diagnosis) of cancer. For example, said methods etc., can be an algorithm that comprises any appropriate combination of the GAG properties as input, to e.g. perform pattern recognition of the samples, in order to arrive at an indication, e.g. in the form of a value or score, which can then be used for screening (e.g. diagnosis) of cancer. Non-limiting examples of such algorithms include machine learning or deep learning algorithms that implement classification (algorithmic classifiers), such as linear classifiers (e.g. Fisher's linear discriminant, logistic regression, naive Bayes classifier, perceptron); support vector machines (e.g. least squares support vector machines); quadratic classifiers; kernel estimation (e.g. k-nearest neighbor); boosting; decision trees (e.g. random forests); neural networks; learning vector quantization.

The use of such classifiers, e.g. machine learning classifiers, e.g. random forest classifiers, would be within the skill of a person skilled in the art. For example, such classifiers can conveniently be trained on GAG properties from a training set of samples and then tested in terms of accuracy on a test set of samples. Classifiers may generate a black-box model that is trained on the most important GAG properties and can thus be used to identify the most important GAG properties which can be used to arrive at accurate screening for (e.g. diagnosis of) cancer.

In some embodiments, a multivariable logistic regression model is used to compute the GAG score. In such embodiment, the GAG score is equal to the log-odds for cancer vs. controls (the response or dependent variable) given one or more GAG properties used as predictors (i.e. explanatory or independent variables or regressors) in the logistic regression model. In some embodiments, one GAG property can be used as explanatory variables. In some embodiments, two or more GAG properties can be used as explanatory variables. In some embodiments, only plasma GAG properties are used as explanatory variables. In such case, a plasma GAG score is derived. In some embodiments, only urine GAG properties are used as explanatory variables. In such case, a urine GAG score is derived. In some embodiments, both plasma and urine GAG properties are used as explanatory variables. In such case, a combined GAG score is derived. The GAG properties used as predictors can be selected from the totally of GAG properties measured in a sample using appropriate feature selection methods. Examples of feature selection methods include filter methods, wrapper methods, embedded methods and are known to of a person skilled in the art. In some embodiments, the multivariable logistic regression model with embedded feature selection is developed using predictive projection modelling (e.g. as per Piironen, J., Paasiniemi, M. and Vehtari, A. ‘Projective inference in high-dimensional problems: Prediction and feature selection’, Electronic Journal of Statistics, 14, 2155-2197 (2020)), available at: https://arxiv.org/abs/1810.02406.). In some embodiments, the response computed from the linear predictor developed using predictive projection modelling is the GAG score. In some embodiments, for a given GAG score, a certain GAG property selected as explanatory variable in the underlying logistic regression model is positively associated with cancer if the corresponding coefficient is positive—conditional to the value of all other GAG property selected as explanatory variables. Conversely, in some embodiments, a certain GAG property selected as explanatory variable in the underlying logistic regression model is negatively associated with cancer if the corresponding coefficient is negative-conditional to the value of all other GAG property selected as explanatory variables.

As described elsewhere herein, screening for cancer in accordance with the present invention may involve using a score (or a GAG score, e.g. a blood (e.g. plasma) GAG score, a urine GAG score or a combined GAG score), or expressing the level and/or chemical composition using a score (or GAG score). As described elsewhere herein, in some such embodiments, an altered score (e.g. increased or decreased as the case may be, in some embodiments preferably increased) in comparison to a control score (or cut-off level or threshold level) is indicative of cancer in said subject.

As indicated above, a preferred cancer in accordance with the invention is prostate cancer (PCa).

In some embodiments, in methods of screening for prostate cancer, particularly preferred GAG forms to be measured or determined in the methods of the invention are one or more (or all) of: Total CS, relative concentration of 4s CS, absolute concentration of 4s CS and relative concentration of 2s6s CS. In some such embodiments, an alteration in the level of one or more (or all) of said GAG forms, e.g. in comparison to a control level, is indicative of prostate cancer.

In some embodiments, in methods of screening for prostate cancer an increase in a blood sample of one or more (or all) of: Total CS, relative concentration of 4s CS and absolute concentration of 4s CS, for example in comparison to a control level, is indicative of prostate cancer in said subject.

In some embodiments, in methods of screening for prostate cancer an increase in a urine sample in the relative concentration of 2s6s CS, for example in comparison to a control level, is indicative of prostate cancer in said subject.

As indicated above, a preferred cancer in accordance with the invention is colorectal cancer (CRC).

In some embodiments, in methods of screening for colorectal cancer, particularly preferred GAG forms to be measured or determined in the methods of the invention are one or more (or all) of: absolute concentration of 0s CS, charge CS and relative concentration of 0s CS. In some such embodiments, an alteration in the level of one or more (or all) of said GAG forms, e.g. in comparison to a control level, is indicative of colorectal cancer.

In some embodiments, in methods of screening for colorectal cancer an increase in a blood sample of one or both of: absolute concentration of 0s CS and relative concentration of 0s CS, for example in comparison to a control level, is indicative of colorectal cancer in said subject.

In some embodiments, in methods of screening for colorectal cancer a decrease in a blood sample in charge CS, for example in comparison to a control level, is indicative of colorectal cancer in said subject.

As indicated above, a preferred cancer in accordance with the invention is a neuroendocrine cancer (e.g. a gastro-instestinal endocrine tumour, GNET).

In some embodiments, in methods of screening for neuroendocrine cancer (e.g. a gastro-instestinal endocrine tumour), particularly preferred GAG forms to be measured or determined in the methods of the invention are one or both of: absolute concentration of 0s CS and Total CS. In some such embodiments, an alteration in the level of one or both of said GAG forms, e.g. in comparison to a control level, is indicative of neuroendocrine cancer (e.g. a gastro-instestinal endocrine tumour).

In some embodiments, in methods of screening for neuroendocrine cancer (e.g. a gastro-instestinal endocrine tumour) an increase in a blood sample of one or both of: absolute concentration of 0s CS and Total CS, for example in comparison to a control level, is indicative of neuroendocrine cancer (e.g. a gastro-instestinal endocrine tumour) in said subject.

As indicated above, a preferred cancer in accordance with the invention is blood cancer (e.g. chronic lymphoid leukaemia, LL; or diffuse large B-Cell lymphoma, NHL).

In some embodiments, in methods of screening for blood cancer (e.g. chronic lymphoid leukaemia or diffuse large B-Cell lymphoma), a particularly preferred GAG form to be measured or determined in the methods of the invention is the absolute concentration of 0s CS. In some such embodiments, an alteration in the level of said GAG form, e.g. in comparison to a control level, is indicative of blood cancer (e.g. chronic lymphoid leukaemia) or diffuse large B-Cell lymphoma).

In some embodiments, in methods of screening for blood cancer (e.g. chronic lymphoid leukaemia or diffuse large B-Cell lymphoma) an increase in a blood sample of the absolute concentration of 0s CS, for example in comparison to a control level, is indicative of blood cancer (e.g. chronic lymphoid leukaemia or diffuse large B-Cell lymphoma) in said subject.

In some embodiments, in methods of screening for chronic lymphoid leukaemia, particularly preferred GAG forms to be measured or determined in the methods of the invention are one or both of: absolute concentration of 0s CS and Total CS. In some such embodiments, an alteration in the level of one or both of said GAG forms, e.g. in comparison to a control level, is indicative of chronic lymphoid leukaemia.

In some embodiments, in methods of screening for chronic lymphoid leukaemia an increase in a blood sample of one or both of: absolute concentration of 0s CS and Total CS, for example in comparison to a control level, is indicative of chronic lymphoid leukaemia in said subject.

In some embodiments, in methods of screening for diffuse large B-Cell lymphoma, particularly preferred GAG forms to be measured or determined in the methods of the invention are one or both of: absolute concentration of 0s CS and Charge CS. In some such embodiments, an alteration in the level of one or both of said GAG forms, e.g. in comparison to a control level, is indicative of diffuse large B-Cell lymphoma.

In some embodiments, in methods of screening for diffuse large B-Cell lymphoma an increase in a blood sample of the absolute concentration of 0s CS, for example in comparison to a control level, is indicative of diffuse large B-Cell lymphoma in said subject.

In some embodiments, in methods of screening for diffuse large B-Cell lymphoma a decrease in a blood sample of Charge CS, for example in comparison to a control level, is indicative of diffuse large B-Cell lymphoma in said subject.

As indicated above, a preferred cancer in accordance with the invention is bladder cancer (BCa).

In some embodiments, in methods of screening for bladder cancer, particularly preferred GAG forms to be measured or determined in the methods of the invention are one or more (or all) of: Total CS, relative concentration of 4s CS, absolute concentration of 4s CS, absolute concentration of 0s CS, absolute concentration of 4s CS, relative concentration of 6s CS and relative concentration of 0s CS. In some such embodiments, an alteration in the level of one or more (or all) of said GAG forms, e.g. in comparison to a control level, is indicative of bladder cancer.

In some embodiments, in methods of screening for bladder cancer an increase in a blood sample of one or more (or all) of: Total CS, relative concentration of 4s CS and absolute concentration of 4s CS, for example in comparison to a control level, is indicative of bladder cancer in said subject.

In some embodiments, in methods of screening for bladder cancer an increase in a urine sample of one or more (or all) of: Total CS, absolute concentration of 0s CS, absolute concentration of 4s CS and relative concentration of 0s CS, for example in comparison to a control level, is indicative of bladder cancer in said subject.

In some embodiments, in methods of screening for bladder cancer a decrease in a urine sample of the relative concentration of 6s CS, for example in comparison to a control level, is indicative of bladder cancer in said subject.

As indicated above, a preferred cancer in accordance with the invention is breast cancer (e.g. breast invasive ductal carcinoma, BC).

In some embodiments, in methods of screening for breast cancer (e.g. breast invasive ductal carcinoma, BC), a particularly preferred GAG form to be measured or determined in the methods of the invention is the absolute concentration of 0s CS, In some such embodiments, an alteration in the level said GAG form, e.g. in comparison to a control level, is indicative of breast cancer.

In some embodiments, in methods of screening for breast cancer (e.g. breast invasive ductal carcinoma, BC) an increase in a blood sample of the absolute concentration of 0s CS, for example in comparison to a control level, is indicative of breast cancer in said subject.

As indicated above, a preferred cancer in accordance with the invention is ovarian cancer (OV).

In some embodiments, in methods of screening for ovarian cancer, particularly preferred GAG forms to be measured or determined in the methods of the invention are one or both of: Total CS and absolute concentration of 0s CS. In some such embodiments, an alteration in the level of one or both of said GAG forms, e.g. in comparison to a control level, is indicative of ovarian cancer.

In some embodiments, in methods of screening for ovarian cancer an increase in a blood sample of one or both of: Total CS and absolute concentration of 0s CS, for example in comparison to a control level, is indicative of ovarian cancer in said subject.

As indicated above, a preferred cancer in accordance with the invention is uterine cancer (e.g. endometrial cancer, EC; or cervical cancer, preferably cervical squamous cell carcinoma, CST).

In some embodiments, in methods of screening for uterine cancer (e.g. endometrial cancer, EC; or cervical cancer, preferably cervical squamous cell carcinoma, CST), particularly preferred GAG forms to be measured or determined in the methods of the invention are one or both of: Total CS and absolute concentration of 0s CS. In some such embodiments, an alteration in the level of one or both of said GAG forms, e.g. in comparison to a control level, is indicative of uterine cancer (e.g. endometrial cancer, EC; or cervical cancer, preferably cervical squamous cell carcinoma, CST).

In some embodiments, in methods of screening for uterine cancer (e.g. endometrial cancer, EC; or cervical cancer, preferably cervical squamous cell carcinoma, CST) an increase in a blood sample of one or both of: Total CS and absolute concentration of 0s CS, for example in comparison to a control level, is indicative of uterine cancer (e.g. endometrial cancer, EC; or cervical cancer, preferably cervical squamous cell carcinoma, CST) in said subject.

As indicated above, a preferred cancer in accordance with the invention is brain tumor (e.g. diffuse glioma, DG).

In some embodiments, in methods of screening for brain tumor (e.g. diffuse glioma, DG), particularly preferred GAG forms to be measured or determined in the methods of the invention are one or both of: Total CS and absolute concentration of 0s CS. In some such embodiments, an alteration in the level of one or both of said GAG forms, e.g. in comparison to a control level, is indicative of brain tumor (e.g. diffuse glioma, DG).

In some embodiments, in methods of screening for brain tumor (e.g. diffuse glioma, DG) an increase in a blood sample of one or both of: Total CS and absolute concentration of 0s CS, for example in comparison to a control level, is indicative of brain tumor (e.g. diffuse glioma, DG) in said subject.

As indicated above, a preferred cancer in accordance with the invention is lung cancer (e.g. non-small cell lung cancer, NSCLC).

In some embodiments, in methods of screening for lung cancer (e.g. non-small cell lung cancer), particularly preferred GAG forms to be measured or determined in the methods of the invention are one or more (or all) of: Total CS, absolute concentration of 0s CS, absolute concentration of 4s CS, absolute concentration of Ns HS, Total HS, absolute concentration of 0s HS, absolute concentration of 2s6s CS, absolute concentration of 4s CS, relative concentration of 6s CS and absolute concentration of 6s CS. In some such embodiments, an alteration in the level of one or more (or all) of said GAG forms, e.g. in comparison to a control level, is indicative of lung cancer (e.g. non-small cell lung cancer).

In some embodiments, in methods of screening for lung cancer (e.g. non-small cell lung cancer) an increase in a blood sample of one or more (or all) of: Total CS, absolute concentration of 0s CS, absolute concentration of 4s CS, for example in comparison to a control level, is indicative of lung cancer (e.g. non-small cell lung cancer) in said subject.

In some embodiments, in methods of screening for lung cancer (e.g. non-small cell lung cancer) an increase in a urine sample of one or more (or all) of: absolute concentration of Ns HS, Total CS, Total HS, absolute concentration of 0s CS, absolute concentration of 0s HS, absolute concentration of 2s6s CS, absolute concentration of 4s CS and absolute concentration of 6s CS, for example in comparison to a control level, is indicative of lung cancer (e.g. non-small cell lung cancer) in said subject.

In some embodiments, in methods of screening for lung cancer (e.g. non-small cell lung cancer) a decrease in a urine sample of the relative concentration of 6s CS, for example in comparison to a control level, is indicative of lung cancer (e.g. non-small cell lung cancer) in said subject.

As indicated above, a preferred cancer in accordance with the invention is kidney cancer (e.g. renal cell cancer, RCC).

In some embodiments, in methods of screening for kidney cancer (e.g. renal cell cancer), particularly preferred GAG forms to be measured or determined in the methods of the invention are one or more (or all) of: Total CS, absolute concentration of 0s CS, relative concentration of 4s CS, absolute concentration of 4s CS, Charge CS, Total HS, relative concentration of 0s CS, absolute concentration of 0s HS and relative concentration of 6s CS. In some such embodiments, an alteration in the level of one or more (or all) of said GAG forms, e.g. in comparison to a control level, is indicative of kidney cancer (e.g. renal cell cancer).

In some embodiments, in methods of screening for kidney cancer (e.g. renal cell cancer) an increase in a blood sample of one or more (or all) of: Total CS, absolute concentration of 0s CS, relative concentration of 4s CS, absolute concentration of 4s CS, for example in comparison to a control level, is indicative of kidney cancer (e.g. renal cell cancer) in said subject.

In some embodiments, in methods of screening for kidney cancer (e.g. renal cell cancer) an increase in a urine sample of one or more (or all) of: Total HS, relative concentration of 0s CS, absolute concentration of 0s CS, absolute concentration of 0s HS, Total CS, for example in comparison to a control level, is indicative of kidney cancer (e.g. renal cell cancer) in said subject.

In some embodiments, in methods of screening for kidney cancer (e.g. renal cell cancer) a decrease in a urine sample of one or both of: Charge CS and the relative concentration of 6s CS, for example in comparison to a control level, is indicative of kidney cancer (e.g. renal cell cancer) in said subject.

As indicated above, a preferred cancer in accordance with the invention is head and neck cancer (e.g. head and neck squamous cell carcinoma, HN).

In some embodiments, in methods of screening for head and neck cancer (e.g. head and neck squamous cell carcinoma), particularly preferred GAG forms to be measured or determined in the methods of the invention are one or more (or all) of: Total CS, absolute concentration of 4s CS, absolute concentration of 0s CS, Total HS, absolute concentration of Ns HS and absolute concentration of 0s HS. In some such embodiments, an alteration in the level of one or more (or all) of said GAG forms, e.g. in comparison to a control level, is indicative of head and neck cancer (e.g. head and neck squamous cell carcinoma).

In some embodiments, in methods of screening for head and neck cancer (e.g. head and neck squamous cell carcinoma) an increase in a blood sample of one or more (or all) of: Total CS, absolute concentration of 4s CS and absolute concentration of 0s CS, for example in comparison to a control level, is indicative of head and neck cancer (e.g. head and neck squamous cell carcinoma) in said subject.

In some embodiments, in methods of screening for head and neck cancer (e.g. head and neck squamous cell carcinoma) an increase in a urine sample of one or more (or all) of: Total HS, absolute concentration of 4s CS, absolute concentration of Ns HS, Total CS and absolute concentration of 0s HS, for example in comparison to a control level, is indicative of head and neck cancer (e.g. head and neck squamous cell carcinoma) in said subject.

As discussed herein, methods of the present invention may comprise determining or measuring one or more specific GAG forms (or groups of GAG forms) “selected from the group consisting of” certain specific GAG forms (or groups of GAG forms) set forth herein. For the avoidance of doubt, in some embodiments in which one or more of the specific GAG forms (or groups of GAG forms) or one or more of the specific genes (or groups of genes) discussed herein is measured or determined, one or more other (or distinct) GAG forms or one or more other (or distinct) genes and/or one or more other biomarkers may additionally be measured or determined. Thus, “selected from the group consisting of” may be an “open” term. In some embodiments, only one or more of the specific GAG forms (or groups of GAG forms) discussed herein is measured or determined (e.g. other GAG forms or other biomarkers are not measured or determined).

As discussed above, the present invention provides a method of screening for cancer in a subject. Alternatively viewed, the present invention provides a method of diagnosing cancer in a subject. Alternatively viewed, the present invention provides a method for the prognosis of cancer in a subject (prognosis of the future severity, course and/or outcome of cancer). Alternatively viewed, the present invention provides a method of predicting for the occurrence of cancer in a subject over a certain time period. Alternatively viewed, the present invention provides a method of estimating the risk of occurrence of cancer in a subject over a certain time period. Alternatively viewed, the present invention provides a method of monitoring for the occurrence of cancer in a subject at risk. Alternatively viewed, the present invention provides a method for monitoring the progression of cancer in a subject. Alternatively viewed, the present invention provides a method of determining the clinical severity of cancer in a subject. Alternatively viewed, the present invention provides a method of determining or estimating the stage of a cancer (e.g. Stage I, Stage II, Stage III or Stage IV cancer). Alternatively viewed, the present invention provides a method of determining or estimating the grade of a cancer (e.g. low grade, or high grade; grade 1 or grade 2 or grade 3 or grade 4). Alternatively viewed, the present invention provides a method of determining the risk of progression of cancer in a subject. Alternatively viewed, the present invention provides a method of guiding treatment based on the risk assessment of cancer in a subject. Alternatively viewed, the present invention provides a method for predicting the response of a subject to therapy for cancer. Alternatively viewed, the present invention provides a method of determining the efficacy of a therapeutic or surgical regime for cancer in a subject. Alternatively viewed, the present invention provides a method for detecting the recurrence or relapse of cancer (e.g. in patients with early stage cancer). Alternatively viewed, the present invention provides a method of patient selection or treatment selection, for example as it provides a means of distinguishing patients with small cancerous masses which are not cancer, e.g. are non-malignant, benign or indolent masses (but which can display some problematic symptoms, and may be suspected to be cancer) from patients with cancer. Thus, alternatively viewed, the present invention provides a method for distinguishing cancer from non-malignant diseases. In some embodiments, the present invention provides a method for determining whether a metastasis is due to a particular cancer. Alternatively viewed, the present invention provides a method for predicting whether metastasis is expected from a given cancer. Alternatively viewed, the present invention provides a method of screening for cancer in the general population. Alternatively viewed, the present invention provides a method of screening for cancer in a population at risk of having or developing cancer (e.g. genetically predisposed individuals or individuals presenting risk factors or individuals presenting symptoms). Alternatively viewed, the present invention provides a method for predicting or determining the tissue of origin of a cancer. Alternatively viewed, the present invention provides a method for estimating the prognosis of a patient with cancer. Alternatively viewed, the present invention provides a method for estimating the prognosis of a patient with a given cancer. Alternatively viewed, the present invention provides a method for predicting or determining whether a cancer is metastatic.

Thus, the method of screening for cancer in accordance with the present invention can be used, for example, for diagnosing cancer, for the prognosis of cancer, for predicting the occurrence of cancer, for estimating the risk of occurrence of cancer, for monitoring for the occurrence of cancer in a subject at risk, for monitoring the progression of cancer, for determining the clinical severity of cancer, for determining or estimating the stage of a cancer (e.g. Stage I, Stage II, Stage III or Stage IV cancer), for determining or estimating the grade of a cancer (e.g. low grade, or high grade; grade 1 or grade 2 or grade 3 or grade 4), for predicting the response of a subject to therapy for cancer, for determining the efficacy of a therapeutic or surgical regime being used to treat cancer, for detecting the recurrence or relapse of cancer, for patient selection or treatment selection, for distinguishing small cancerous masses suspicious of cancer from other non malignant diseases, for determining whether a metastasis is due to a given cancer, for screening for cancer in the general population, for screening for cancer in a population at risk of having or developing cancer (e.g. genetically predisposed individuals or individuals presenting risk factors or individuals presenting symptoms), for predicting or determining whether a cancer is metastatic, or for predicting or determining the tissue of origin of a cancer.

Thus, in one aspect the present invention provides a method for diagnosing cancer in a subject. In some embodiments, a positive diagnosis (i.e. the presence of cancer) is made if the level of one or more of the GAG forms in the sample is altered (increased or decreased as the case may be) in comparison to a control level. GAG forms for which an increased level is indicative of (e.g. diagnostic of) cancer are described elsewhere herein. GAG forms for which a decreased level is indicative of (e.g. diagnostic of) cancer (e.g. prostate cancer) are described elsewhere herein. Alternatively, a number of different GAG forms or properties are analysed as described elsewhere herein to arrive at a diagnosis, e.g. using a scoring system or method (e.g. a GAG score). When a scoring system (e.g. a GAG score) is used, a positive diagnosis (i.e. the presence of cancer) may be made if the score is altered (or significantly altered) in comparison to a control score or cut-off (or threshold) level.

In another aspect, the present invention provides a method for the prognosis of cancer in a subject. In such methods the level of one or more of the GAG forms (or a score) discussed above in the sample is indicative of the future severity, course and/or outcome of cancer. For example, an alteration (increase or decrease as the case may be) in the level of one or more of the GAG forms in the sample (or a score) in comparison to a control level (or control score or cut-off level) may indicate a poor prognosis. A highly altered level (or score if a score is used), e.g. compared to control levels (or scores or cut-off levels), may indicate a particularly poor prognosis. Thus, in some embodiments, an increased level of one or more of the GAG forms for which an increased level is indicative of cancer is suggestive of a poor prognosis. In some embodiments, a decreased level of one or more of the GAG forms for which a decreased level is indicative of cancer is suggestive of (i.e. indicative of) a poor prognosis. Conversely, if one or more GAG forms has an unaltered level (or an essentially unaltered level) that can be indicative of a good prognosis. In some embodiments, when a scoring system is used, an increased score for which an increased score is indicative of cancer is suggestive of a poor prognosis. In some embodiments, when a scoring system is used, a decreased score for which a decreased score is indicative of cancer is suggestive of (i.e. indicative of) a poor prognosis. Conversely, if one or more GAG scores is unaltered (or essentially unaltered) that can be indicative of a good prognosis.

Serial (periodic) measuring of the level of one or more of the GAG forms (biomarkers) in accordance with the present invention may also be used for prognostic purposes looking for either increasing or decreasing levels (or scores) over time. In some embodiments, an altering level or score (increase or decrease, as appropriate) of one or more of the GAG forms or scores over time (in comparison to a control level or score or cut-off level, e.g. a level or score moving further away from the control level or score or cut-off level) may indicate a worsening prognosis. In some embodiments, an altering level (increase or decrease, as appropriate) of one or more of the GAG forms or scores over time (in comparison to a control level or score or cut-off level, e.g. a level moving closer to the control level or score or cut-off level) may indicate an improving prognosis.

In some embodiments, an alteration (an increase or decrease as the case may be) in the level and/or chemical composition of one or both of the glycosaminoglycans (GAGs) chondroitin sulfate (CS) and heparan sulfate (HS) in a body fluid sample (or score) (e.g. in comparison to a control level or composition or score), may be indicative of prognosis.

Thus, prognostic methods of the invention may be used to predict (or estimate) the prognosis, e.g. whether a subject has a good (or a better) prognosis or whether a subject has a poor (or worse) prognosis. In some embodiments, an alteration (an increase or decrease as the case may be) in the level and/or chemical composition of one or both of the glycosaminoglycans (GAGs) chondroitin sulfate (CS) and heparan sulfate (HS) in a body fluid sample (or score) may be indicative of a good (or better) prognosis in relation to the prognosis for a control subject or control population (or average (e.g. median) control subject or level in a control or reference population) from which a control level (or score) was obtained (or derived). In some embodiments, an alteration (an increase or decrease as the case may be) in the level and/or chemical composition of one or both of the glycosaminoglycans (GAGs) chondroitin sulfate (CS) and heparan sulfate (HS) in a body fluid sample (or score) may be indicative of a poor (or worse) prognosis in relation to the prognosis for a control subject or control population (or average (e.g. median) control subject or level in a control or reference population) from which the control level (or score) was obtained (or derived).

Prognosis may be considered as an assessment of the survival prospects for a subject (e.g. over a given time period, e.g. 3, 6, 9, 12, 15, 18, 21, 24, 27, 30 or 33 months, or e.g. 5-year survival). Thus, alternatively viewed, the present invention provides in one aspect a method of predicting (or determining) the survival prospects for a subject having cancer. In some embodiments, “survival” may be calculated as the time (e.g. in days) between the date of sampling (the date a sample is obtained from a subject) and the time of an “event”. In some embodiments, “survival” may be calculated from start of cancer treatment, e.g the start of pharmaceutical therapy or the day of surgery. Alternatively viewed, prognosis may be considered as an assessment of the mortality prospects or risk of progression to a worse condition for a subject. Thus, the present invention provides in one aspect a method of predicting (or determining) the risk of death or of progression for a subject having cancer.

In one embodiment, “survival” is “overall survival” (OS). In such embodiments, the “event” is the date of death. Thus, in some embodiments, prognostic methods of the invention may be used to predict (or estimate) the probability of death occuring in a particular future time period (e.g. 3, 6, 9, 12, 15, 18, 21, 24, 27, 30 or 33 months, or e.g. 5-year survival). In some embodiments, prognostic methods of the invention may be used to predict or estimate the amount of time before death. In some embodiments, “survival” is “progression-free survival” or “disease-free survival” or “recurrence-free survival” or “treatment-free survival”.

In one aspect the present invention provides a method for monitoring for the occurrence of cancer in a subject at risk of developing cancer. Such methods and the GAG forms (or scores) which are measured are similar to the diagnostic methods as described herein, but are carried out on subjects that are at particular risk for developing cancer and thus may benefit from closer monitoring. Such “at risk” subjects would be readily identified by a person skilled in the art but would include for example subjects with a family history of cancer or a genetic predisposition to cancer, or subjects in remission from cancer, or subjects with recognized risk factors for cancer. For example, for some cancers, a recognized risk factor for is age, for example over 40 years old, or over 50 years old, or over 55 years old, or preferably over 65 years old, e.g. 40-65, or 50-65, or 55-65, or 60-65, or 70-75, or 40-75, or 50-75, or 55-75, or 60-75, or 65-75 (e.g. 66-71), or 70-75, or 40-85, or 50-85, or 55-85, or 60-85, or 65-85, or 70-85. In prostate cancer for example, males in such age ranges may be particularly at risk.

In this way, it can be seen that in some embodiments of the invention, the methods can be carried out on “healthy” patients (subjects) or at least patients (subjects) which are not manifesting any clinical symptoms of cancer, for example, patients with very early or pre-clinical stage cancer, e.g. patients where the primary tumor is so small that it cannot be assessed or detected or patients in which cells are undergoing pre-cancerous changes associated with cancer but have not yet become malignant. Thus, in another aspect the present invention provides a method to predict the occurrence of cancer in a subject (e.g. over a given time period, e.g. 3, 6, 9, 12, 15, 18, 21, 24, 27, 30, 33, 36, 39, 42, 45, 48, 51, 54, 57 or 60 months). In another aspect the present invention provides a method to estimate the risk of occurrence of cancer in a subject (e.g. over a given time period, e.g. 3, 6, 9, 12, 15, 18, 21, 24, 27, 30, 33, 36, 39, 42, 45, 48, 51, 54, 57 or 60 months).

Thus, the methods of the present invention can also be used to monitor disease progression. Such monitoring can take place before, during or after treatment of cancer by surgery or therapy, e.g. pharmaceutical therapy. Thus, in another aspect the present invention provides a method for monitoring the progression of cancer in a subject. In some embodiments of methods for monitoring the progression of cancer (e.g. kidney cancer or other cancer described herein) in a subject, the level of one or more of the GAG forms (or a score or a composition) is indicative of the progression of cancer. In some preferred embodiments, the level of the absolute concentration of 0s CS (or a chemical composition determined that includes the absolute concentration of 0s CS or a score used that uses (or comprises) the absolute concentration of 0s CS) is indicative of cancer progression, preferably with high (or higher) or increased (or increasing) levels (e.g. as measured over time e.g. by taking serial measurements) being indicative of cancer progression (cancer worsening).

Methods of the present invention can also be used to predict (or determine) whether a particular cancer (e.g. kidney cancer or other cancer discussed herein) is metastatic. In some preferred embodiments, the level of the absolute concentration of 0s CS (or a chemical composition determined that includes the absolute concentration of 0s CS or a score used that uses (or comprises) the absolute concentration of 0s CS) is indicative of metastasis, preferably with high (or higher) or increased (or increasing) levels (e.g. as measured over time e.g. by taking serial measurements) being indicative of metastatis. Thus, in some embodiments, the absolute concentration of 0s CS (e.g. in a blood, e.g. plasma, sample, or in a urine sample) may be indicative of metastatic cancer.

Methods of the present invention can be used in the active monitoring of patients which have not been subjected to surgery or therapy, e.g. to monitor the progress of cancer in untreated patients. Again, serial measurements can allow an assessment of whether or not, or the extent to which, the cancer is worsening, thus, for example, allowing a more reasoned decision to be made as to whether therapeutic or surgical intervention is necessary or advisable.

As discussed above, monitoring can also be carried out, for example, in an individual, e.g. a healthy individual, who is thought to be at risk of developing cancer, in order to obtain an early, and ideally pre-clinical, indication of cancer.

In another aspect, the present invention provides a method for determining the clinical severity of cancer in a subject. In such methods the level of one or more of the GAG forms in the sample (or a score derived therefrom) shows an association with the severity of the cancer. Thus, the level of one or more of the GAG forms (or a score) is indicative of the severity of the cancer. In some embodiments, the more altered (more increased or more decreased as the case may be) the level of one or more of the GAG forms (or score derived therefrom) in comparison to a control level (or score), the greater the likelihood of a more severe form of cancer. In some embodiments the methods of the invention can thus be used in the selection of patients for therapy.

Serial (periodical) measuring of the level of one or more of the GAG forms (biomarkers) (or score derived therefrom) may also be used to monitor the severity of cancer looking for either increasing or decreasing levels over time. Observation of altered levels (increase or decrease as the case may be) may also be used to guide and monitor therapy, both in the setting of subclinical disease, i.e. in the situation of “watchful waiting” before treatment or surgery, e.g. before initiation of pharmaceutical therapy or surgery, or during or after treatment to evaluate the effect of treatment and look for signs of therapy failure.

Thus, the present invention also provides a method for predicting the response of a subject to therapy or surgery. For example, a subject with a less severe form or an early stage of cancer (e.g. prostate cancer), as determined by the level of one or more of the GAG forms in a sample in accordance with the present invention (or a score derived therefrom), is generally more likely to be responsive to therapy or surgery, in particular surgery. In such methods the choice of therapy or surgery may be guided by knowledge of the level of one or more of the GAG forms in the sample (or the score derived therefrom). In some embodiments of methods for predicting the response of a subject to therapy or surgery, the level of HA is not measured or determined.

The present invention also provides a method of patient selection or treatment selection as it provides a means of distinguishing patients with high risk cancer from patients with low risk cancer. Thus, alternatively viewed, the methods of the present invention provide a method for distinguishing high and low risk cancer and may guide appropriate treatment.

In some embodiments, the invention provides a method of distinguishing between high (or higher) risk cancer (e.g. prostate cancer e.g. with a Gleason score of 8 or more) and low (or lower) risk cancer (e.g. prostate cancer e.g. with a Gleason score of 6 or less) in subjects that have been diagnosed (e.g. recently diagnosed e.g. <1 month or <6 months or <1 year since diagnosis) with cancer (e.g. prostate cancer). High (or higher) risk may mean a subject has a poor (or worse) prognosis and low (or lower) risk may mean a subject has a good (or better) prognosis. Subjects of intermediate risk (e.g. prostate cancer subjects with a Gleason score of 7) may also be identified. Methods of the invention may be used to assess the severity, aggressiveness, metastatic potential or risk level in a subject diagnosed (e.g. recently diagnosed) with cancer (e.g. prostate cancer).

In some embodiments, the invention provides a method of monitoring (e.g. continuously monitoring or performing active surveillance of) a subject having cancer (e.g. a subject being treated for cancer). Such monitoring may guide which treatment to use or whether no treatment should be given.

In some embodiments, the more altered (more increased or decreased as the case may be) the level (or score) of one or more of the GAG forms in comparison to a control level (or score or cut-off level), the greater the likelihood of high risk cancer (or the lesser the likelihood of low risk cancer). Conversely, in some embodiments, the less altered (less increased or decreased as the case may be) the level (or score) of one or more of the GAG forms in comparison to a control level (or score cut-off level), the lesser the likelihood of high risk cancer (or the greater the likelihood of low risk cancer).

In some embodiments, low (or lower) risk patients may be put under watchful waiting or active surveillance and may not be given treatment (e.g. pharmaceutical therapy or surgery). In some embodiments, high (or higher) risk patients may be given treatment, e.g. resection (e.g. prostatectomy in the case of prostate cancer), radiation therapy, hormone therapy or other treatment (e.g. as detailed elsewhere herein).

The present invention also provides a method of determining (or monitoring) the efficacy of a therapeutic regime being used to treat cancer, in other words following or monitoring a response to treatment. In such methods, an alteration (increase or decrease as the case may be) in the level (or scores) of one or more of the GAG forms in accordance with the present invention indicates the efficacy of the therapeutic regime being used. For example, if the level of one or more of the GAG forms (or a score derived therefrom (or based thereon)) for which an increased level (or score) is indicative of cancer is reduced during (or after) therapy, this is indicative of an effective therapeutic regime. Conversely, for example, if the level of one or more of the GAG forms (or a score derived therefrom) for which a decreased level (or score) is indicative of cancer is increased during (or after) therapy, this is indicative of an effective therapeutic regime. In such methods, serial (periodical) measuring of the level of one or more of the GAG forms (biomarkers) over time can also be used to determine the efficacy of a therapeutic regime being used. Similar methods can be used to provide a method of determining (or monitoring) the efficacy of a surgical regime being used to treat cancer.

The present invention also provides a method for detecting the recurrence (relapse) of cancer, for example in a subject that has previously had cancer but been successfully treated, e.g. by surgery or therapy (e.g. pharmaceutical therapy) such that they are judged to be in remission or cured, or for example to predict metastatic relapse in patients during follow-up. Such subjects form an “at risk” category and may well benefit from regular monitoring for cancer. Such methods for detecting the recurrence (or relapse) of cancer use the diagnostic methods as described herein in order to detect the presence or absence of cancer.

The present invention also provides a method of patient selection or treatment selection as it provides a means of distinguishing patients with cancer from patients with non-malignant diseases, e.g. non-malignant prostate diseases. Thus, alternatively viewed, the methods of the present invention provide a method for distinguishing cancer from non-malignant diseases.

Such methods of patient selection or treatment selection or methods for distinguishing cancer from non-malignant diseases use the diagnostic methods as described herein in order to detect the presence or absence of cancer.

The present invention also provides a method of predicting or determining the tissue of origin of a cancer. In some such methods the level and/or composition of one or more of the GAG forms described herein (or a score) may be indicative of the tissue of origin of cancer. Predicting or determining a cancer's tissue of origin may guide further diagnostic and/or therapeutic and/or surgical follow-up (e.g. by recommending a particular type of scan (e.g. CT scan or MRI scan) on a particular part of the body (e.g. chest CT scan versus abdominal CT scan versus brain MRI scan)). Predicting or determining tissue of origin may be done as described in the Example section herein. Predicting or determining tissue of origin may employ any suitable modelling tool or modelling method using the levels of measured GAG forms as inputs, e.g. a multiclass classification machine learning method could be used with the levels of measured GAG forms as inputs. Non-limiting examples of machine learning methods are linear classifiers (e.g. Fisher's linear discriminant, logistic regression, naive Bayes classifier, perceptron); support vector machines (e.g. least squares support vector machines); quadratic classifiers; kernel estimation (e.g. k-nearest neighbor); boosting; decision trees (e.g. random forests); neural networks; deep neural networks; learning vector quantization.

The features and discussion herein in relation to the method of screening for cancer (e.g. in relation to preferred GAG forms or combinations thereof or scores for measurement) apply, mutatis mutandis, to the other related methods of present invention (e.g. to a method of diagnosing cancer, etc.).

In some embodiments, the invention provides the use of the methods of the invention (e.g. screening, diagnostic or prognostic methods, etc., as described herein) in conjunction with other known screening, diagnostic or prognostic methods for cancer, such as radiological imaging (e.g. computed tomography, CT, or positron emission tomography, PET, scan) or magnetic resonance imaging (MRI scan), or histological assessment, e.g. using a tumor biopsy. By way of an example, in the case of prostate cancer, the PSA (prostate specific antigen) test or the DRE (digital rectal examination) test or the Prostate Core Mitomic Test™ may be used in conjunction with a method of the present invention. Thus, for example, the methods of the invention can be used to confirm a diagnosis of cancer in a subject. In some embodiments the methods of the present invention are used alone.

The level of the GAG form in question can be determined or measured by analyzing the sample which has been obtained from or removed from the subject by an appropriate means. The determination is typically carried out in vitro.

Levels of one or more of the GAG forms in the sample can be measured (determined) by any appropriate assay or technique or method, a number of which are well known and documented in the art. Electrophoresis, e.g. agarose gel electrophoresis or capillary electrophoresis (in particular capillary electrophoresis with fluorescence detection such as CE-LIF) is a technique that can be used for measuring (determining) the levels of one or more of the GAG forms in accordance with the invention. Liquid chromatography, in particular HPLC (high-performance liquid chromatography) in combination with mass spectrometry (MS) are preferred techniques for measuring (determining) the levels of one or more of the GAG forms in accordance with the present invention.

Suitable electrophoresis, e.g. capillary electrophoresis, and liquid chromatography, e.g. HPLC techniques for GAG form analysis, together with appropriate mass spectrometry methods (and associated data processing techniques) are well known and documented in the art.

One method that may be used in the invention is capillary electrophoresis with laser-induced fluorescence detection, CE-LIF (e.g. as described in Galeotti et al., 2014, Electrophoresis 35: 811-818; and Kottler et al., 2013, Electrophoresis 34: 2323-2336). HPLC combined with post column derivatization and fluorimetric detection can also be used, e.g. as described in Volpi 2006, Curr Pharm Des 12:639-658, as can HPLC combined with ESI-MS (electrospray ionization-mass spectrometry), e.g. as described in Volpi and Linhardt, 2010, Nature protocols 5:993-1004, also with fluorimetric detection, e.g. as described in Galeotti and Volpi, 2011, Anal Chem 83:6770-6777, or Volpi et al., 2014, Nature Protocols 9:541-558. Agarose gel electrophoresis can also be used, e.g. FACE (fluorophore assisted carbohydrate electrophoresis) as described in Volpi and Maccari, 2006, Analyt Technol Biomed Life Sci, 834:1-13; and Volpi and Maccari, 2002, Electrophoresis 23:4060-4066. A particularly preferred method for determining the level of one or more of the GAG forms in the sample is described herein in the Examples. Thus, preferred methods may involve high performance liquid chromatography (HPLC), preferably ultra-HPLC (UHPLC), in combination with mass spectrometry, e.g. MS/MS or triple quadropole mass spectrometry. Particularly preferred methods comprise ultra-high-performance liquid chromatography (UHPLC) coupled with electrospray ionization triple-quadrupole mass spectrometry system. An example of such methods is described in Tamburro et al., 2012, bioRxiv, doi: 10.1101/2021.02.04.429694.

Certain methods of sample preparation (or processing), e.g. GAG extraction and purification are also known and described in the art, for example Volpi and Maccari, 2005, Biomacromolecules 6:3174-3180 and Clin Chim Acta 356: 125-133, Coppa et al., 2011 Glycobiology 21:295-303. However, such reported art based methods of sample preparation (or processing) involve a protease treatment (protease extraction) and purification step based on using an anion-exchange resin. In some methods of the present invention such a protease treatment step and/or purification step using an anion-exchange resin may be performed. However, as discussed elsewhere herein, in preferred methods a step of protease treatment and/or a purification step using an anion-exchange resin is not performed. In particular, in preferred methods where the protein-free fraction of the GAGs is analysed, then a step of protease treatment is not performed.

In some embodiments HPLC and mass spectrometry (and associated data processing techniques) is used to obtain a fraction of the level of one or more particular GAG forms (e.g. the sulfated or unsulfated disaccharide forms) in the sample in comparison to the total amount. For example, after sample preparation, GAGs can be digested using enzymes, separated in an HPLC column and characterized using MS. As described elsewhere herein, the quantities of one or more individual GAG forms (e.g. a particular sulfated or unsulfated disaccharide form) may be conveniently normalised (i.e. divided) by the sum of all the quantities of individual GAG forms measured, to yield fractions (or proportions or relative concentrations). However, absolute concentrations (or absolute levels) of individual GAG forms (e.g. GAG sulfation forms) may alternatively, or additionally, be measured.

In accordance with the present invention, a quantitative, semi-quantitative or qualitative assessment (determination) of the level of one or more of the GAG forms can be made.

Appropriate methods of doing this would be well known to a skilled person in the art and any of these could be used. However, a convenient method to achieve such quantification of disaccharide composition or the appropriate properties or forms of CS or HS (and separation of the disaccharide forms) is to use electrophoresis, in particular capillary electrophoresis, e.g. capillary electrophoresis with fluorescence detection, e.g. capillary electrophoresis with laser-induced fluorescence detection (CE-LIF) (e.g. as described in Galeotti 2014, supra, or Kottler 2013, supra). An alternative method, which is preferred in some embodiments, is to use liquid chromatography, preferably HPLC (high-performance liquid chromatography), for example SAX HPLC or for example as described in Volpi 2006, supra, Galeotti and Volpi 2011, supra, Volpi et al., 2014, supra or Volpi and Linhardt, 2010, supra. Preferably mass spectrometry is also used (HPLC-MS), for example electrospray ionization mass spectrometry (ESI-MS), e.g. HPLC ESI-MS. Particularly preferred methods are outlined in the Examples. One example of a particular method is capillary electrophoresis (e.g. for example, capillary electophoresis with laser-induced fluorescence detection). Another particularly preferred example would be HPLC followed by MS (HPLC-MS), e.g. HPLC ESI-MS. Preferred HPLC-MS methods are discussed elsewhere herein.

Thus, in preferred methods of the invention said level or chemical composition of said GAG or GAG property is determined by HPLC and mass spectrometry. Preferably said HPLC is ultra-HPLC and/or said mass spectrometry is triple-quadropole mass spectrometry. In certain preferred methods, said level or chemical composition of said GAG or GAG property is determined by high performance liquid chromatography (HPLC), preferably ultra-HPLC (UHPLC), in combination with mass spectrometry, e.g. MS/MS or triple quadropole mass spectrometry. Preferred methods comprise ultra-high-performance liquid chromatography (UHPLC) coupled with (or in combination with) electrospray ionization triple-quadrupole mass spectrometry.

Generally, the determination of the GAG properties or forms in accordance with the present invention does not involve the measurement of GAG molecules in the exact same form as found in the body fluid of a subject (e.g. does not involve the measurement of a naturally occurring form of GAG). For example, such native or naturally occurring GAG molecules are often found in biological samples, e.g. body fluid samples, in the form of long sugar chains which can either be attached to proteins (also referred to herein as protein-bound GAGs or proteoglycan GAGs), or not attached to proteins (also referred to herein as free GAGs or protein-free GAGs).

In some embodiments, methods of the invention may include a step of processing a sample. In some embodiments, the methods of the invention may thus be performed on such processed samples or materials derived from such processed samples. Thus, generally the methods of the invention are carried out on samples which have been processed in some way (e.g. are man-made rather than native samples).

Processing steps include, but are not limited to, extraction or purification of GAGs from the sample, steps of fragmentation or cleavage or digestion of proteins present in the sample, e.g. as a means of separating or extracting or removing GAGs from the protein to which they are attached, e.g. through the use of a protease such as proteinase K, purification of GAGs, e.g. using an anion-exchange resin, isolating cells from the sample, isolating cell components from the sample, extracting (e.g. isolating or purifying) proteins/peptides from the sample. Said processing steps thus also include steps carried out on a body fluid sample to prepare it for analysis, e.g. in the case of a blood sample, such steps might include the steps to prepare an appropriate blood component for analysis, e.g. plasma or serum, or, in the case of a urine sample, the removal of cells or other impurities. A processing step may involve one or more of digestion, extraction, purification, boiling, filtration, lyophilization, fractionation, centrifugation, concentration, dilution, inactivation of interfering components, addition of reagents, derivatization, complexation and the like. Exemplary processing steps are described in the Examples.

Although in certain methods of the invention steps of fragmentation or cleavage or digestion of proteins present in the sample (e.g. as a means of separating or extracting or removing GAGs from the protein to which they are attached, e.g. through the use of a protease such as proteinase K) and/or purification of GAGs (e.g. using an anion-exchange resin) may be done, as is evident from the discussion elsewhere herein in certain preferred methods a step of fragmentation/cleavage/digestion of proteins and/or a step of purification (e.g. using an anion-exchange resin) is not performed. In particular, in preferred methods where the level and/or chemical composition of the protein-free fraction of the GAGs is determined, a step of fragmentation/cleavage/digestion of proteins is not performed.

In general, the GAG containing body fluid sample that has been obtained from a subject is subjected to at least one processing step prior to determining the level and/or chemical composition in accordance with the methods of the present invention. In particular, in methods wherein the levels of one or more of the specific sulfated or unsulfated forms of CS or HS disaccharides are determined, the GAGs are preferably subjected to a processing step to obtain the disaccharide units for analysis.

In some such methods of the invention where the levels of certain individual disaccharide forms are measured, the GAGs, e.g. the full length GAG molecules, or polymerised polysaccharide chains of GAGs, or chains of repeating disaccharide units of GAGs, are subjected to a processing step, for example a step of fragmentation or cleavage or digestion, e.g. by chemical digestion or enzyme treatment. Appropriate methods of digestion or enzyme treatment would be known to a person skilled in the art, e.g. the use of one or more GAG lyase enzymes, e.g. one or more chondroitinase enzymes such as Chondroitinase ABC or Chondroitinase B, and/or the use of one or more heparinase enzymes such as Heparinase I-II-III, in order to obtain the disaccharide units which are then analysed.

Other methods to determine levels or compositions of GAGs which might be used are known in the art. However, examples are analytical techniques involving the use of antibodies to various GAG forms, e.g. techniques such as Western blot, ELISA or FACS, or methods involving agarose gel electrophoresis (e.g. fluorophore-assisted carbohydrate electrophoresis (FACE)) or polyacrylamide gel electrophoresis (PAGE). In some embodiments, the level of one or more GAG forms (e.g. specific sulfated or unsulfated forms of CS or HS disaccharides, which have for example been derived from the full length GAG molecule or a chain of repeating disaccharide units of a GAG molecule by fragmentation, cleavage or digestion) in association with (e.g. physical association with or in complex with or derivitized with or labelled with) a reagent (e.g. 2-aminoacridone) that is being used to detect the GAG form is determined. Thus, in some embodiments the level of a complex of a GAG form and the reagent used to detect the GAG form is determined. Reagents suitable for detecting particular GAG forms are discussed elsewhere herein, but include antibodies, or some kind of fluorophore (or other detectable label or dye) attached to (or used to derivitize) the GAG form in question, for example to make it detectable by a fluorimeter (or other detection device). Thus, purely by way of example, in some embodiments the level of a GAG form in association with (e.g. in complex with or derivitized with) an antibody or fluorophore or the like may be determined. In some embodiments the level of a GAG form in association with (e.g. in complex with or derivitized with) 2-aminoacridone may be determined.

As preferred methods of the invention comprise the step of determining the level and/or chemical composition of the protein-free fraction of one or both of the glycosaminoglycans (GAGs) chondroitin sulfate (CS) and heparan sulfate (HS) in a body fluid sample, advantageously there is no need for the GAG molecules to be separated or extracted from the proteins to which they are attached. Instead, the protein-free fraction (only the protein-free fraction) of the GAGs in a body fluid sample can be analysed from the sample without any need for such processing to separate the GAGs from the protein, e.g. by digesting the protein. Thus, preferred methods do not comprise a processing step in which said samples are contacted with a proteolytic agent such as a protease.

Other preferred methods do not comprise a step in which the GAGs are purified from the sample based on the negative charge of said GAGs (e.g. using an anion-exchange resin).

Thus, in preferred methods of the present invention, said sample has been obtained from said subject and has been subjected to processing prior to determining said level and/or chemical composition,
wherein said processing
   (a) comprises fragmenting said one or both GAGs into disaccharide units; and
   (b) does not comprise prior to (a) at least one of:
      (i) contacting said sample with a proteolytic agent; and
      (ii) purifying said one or both GAGs in said sample based on the negative charge of said GAGs.

A yet further aspect of the invention provides a method of screening for cancer in a subject, said method comprising determining the level and/or chemical composition of one or both of the glycosaminoglycans (GAGs) chondroitin sulfate (CS) and heparan sulfate (HS) in a body fluid sample,
wherein said sample has been obtained from said subject and has been subjected to processing prior to determining said level and/or chemical composition,
   wherein said processing
      (a) comprises fragmenting said one or both GAGs into disaccharide units; and
      (b) does not comprise prior to (a) at least one of:
         (i) contacting said sample with a proteolytic agent; and
         (ii) purifying said one or both GAGs in said sample based on the negative charge of said GAGs.

Embodiments of other aspects of the invention described elsewhere herein may be applied, mutatis mutandis, to this aspect of the invention (e.g. preferred GAG forms (or groups of GAG forms), preferred cancers (or groups of cancers), preferred processing steps, preferred body fluids, etc.).

In preferred methods, said fragmenting of (a) is conveniently performed by contacting said one or both GAGs with one or more GAG lyase enzymes (e.g. as discussed elsewhere herein). For example, said fragmenting of (a) may be performed by contacting said one or both GAGs with one or more chondroitinase enzymes and/or one or more heparinase enzymes.

In art based methods, said contacting step of (b)(i) is conveniently performed by contacting said sample with one or more protease enzymes, e.g. proteinase K. Thus, in preferred methods of the invention such a step is not carried out. The proteolytic agent of (b)(i) may be a protease (e.g. a non-specific protease such as proteinase K). Thus, preferably methods of the invention do not comprise prior to (a) a step of contacting the sample with a protease (e.g. a non-specific protease e.g. proteinase K).

In art based methods, said purifying step of (b)(ii) is conveniently performed by using an anion-exchange resin. Thus, in preferred methods of the invention such a step is not carried out. Thus, preferably methods of the invention do not comprise prior to (a) a step of purifying said one or both GAGs in said sample using an anion-exchange resin.

In preferred methods of the invention, the method does not comprise the contacting of (b)(i).

In preferred methods of the invention, the method does not comprise the purifying of (b)(ii).

In other preferred methods of the invention neither step (b)(i) nor (b)(ii) are carried out. Thus, in particularly preferred embodiments the method does not comprise the contacting of (b)(i) or the purifying of (b)(ii).

A yet further aspect of the invention provides a method of screening for cancer in a subject, said method comprising determining the level and/or chemical composition of the non-proteoglycan fraction of one or both of the glycosaminoglycans (GAGs) chondroitin sulfate (CS) and heparan sulfate (HS) in a body fluid sample, wherein said sample has been obtained from said subject. Embodiments of other aspects of the invention described elsewhere herein may be applied, mutatis mutandis, to this aspect of the invention (e.g. preferred GAG forms (or groups of GAG forms), preferred cancers (or groups of cancers), preferred processing steps, preferred body fluids, etc.).

A yet further aspect of the invention provides a method of screening for cancer in a subject, said method comprising determining the level and/or chemical composition of one or both of the glycosaminoglycans (GAGs) chondroitin sulfate (CS) and heparan sulfate (HS) in a body fluid sample, wherein said determining the level and/or chemical composition comprises analysing disaccharide units that have been derived from said GAGs that are non-protein bound in said body fluid sample and does not comprise analysing disaccharide units that have been derived from GAGs that are protein-bound in said body fluid sample. Embodiments of other aspects of the invention described elsewhere herein may be applied, mutatis mutandis, to this aspect of the invention (e.g. preferred GAG forms (or groups of GAG forms), preferred cancers (or groups of cancers), preferred processing steps, preferred body fluids, etc.).

A yet further aspect of the invention provides a method of screening for cancer in a subject, said method comprising determining the level and/or chemical composition of one or both of the glycosaminoglycans (GAGs) chondroitin sulfate (CS) and heparan sulfate (HS) in a body fluid sample, wherein said determining the level and/or chemical composition comprises analysing a population of disaccharide units consisting essentially of disaccharide units that have been derived from the non-proteoglycan fraction (or protein-free fraction) of said GAGs in said body fluid sample. Embodiments of other aspects of the invention described elsewhere herein may be applied, mutatis mutandis, to this aspect of the invention (e.g. preferred GAG forms (or groups of GAG forms), preferred cancers (or groups of cancers), preferred processing steps, preferred body fluids, etc.).

In another aspect, the present invention provides a method of screening for cancer in a subject, said method comprising determining the level and/or chemical composition of one or both of the glycosaminoglycans (GAGs) chondroitin sulfate (CS) and heparan sulfate (HS) in a body fluid sample, wherein said sample has been obtained from said subject. In preferred embodiments an altered level and/or chemical composition of chondroitin sulfate (CS) and/or heparan sulfate (HS) in said sample in comparison to a control level and/or chemical composition is indicative of cancer in said subject. Embodiments of other aspects of the invention described elsewhere herein may be applied, mutatis mutandis, to this aspect of the invention (e.g. GAG forms (or groups of GAG forms), cancers (or groups of cancers), processing steps, body fluids, fractions of GAGs to analyse, types of method, etc.). Any features (e.g. preferred features) described elsewhere herein in relation to any other aspects of the invention may be applied, mutatis mutandis, to this aspect of the invention.

Thus, although in some embodiments of certain preferred methods of the invention the method comprises determining the level and/or chemical composition of the protein-free fraction of one or both of the glycosaminoglycans (GAGs) chondroitin sulfate (CS) and heparan sulfate (HS), it is not essential in all aspects of the invention for the level and/or chemical composition specifically of the “protein-free fraction” to be determined.

In some embodiments, the present invention provides a method of screening for cancer in a subject, said method comprising determining the absolute concentration of 0s CS in a body fluid sample (e.g. a blood, e.g. plasma, sample or a urine sample), wherein said sample has been obtained from said subject. In preferred embodiments an altered absolute concentration of 0s CS, preferably an increased absolute concentration of 0s CS, in said sample in comparison to a control level is indicative of cancer in said subject. Preferred cancers are described elsewhere herein.

In some embodiments, the only GAG form used for the basis of the screening is 0s CS, (i.e. in some embodiments the level of a single GAG form (GAG property) is used for the basis of the screening for cancer, e.g. a diagnosis, prognosis may be made on the basis of the level of a single GAG form, and that single GAG fom is the absolute concentration of 0s CS). In other embodiments, more than one (e.g. 2, 3, 4, 5, 6, 7, 8, 9, 10, 11, 12, 13, etc.) GAG form is used for the basis of the screening for cancer, e.g. a diagnosis, prognosis may be made on the basis of the level of more than one GAG form in some embodiments wherein one GAG form used is the absolute concentration of 0s CS, e.g. in some embodiments screening may be on the basis of one of any one of the groups (or sub-groups) of GAG forms set out herein that comprise the absolute concentration of 0s CS. In some embodiments, where one GAG form or a group (or sub-group or subset) of GAG forms is used for the basis of the screening for cancer (e.g. diagnosis or prognosis), the level of one or more (or all) of the other GAG forms (or GAG properties) described herein may be additionally determined or measured.

An altered level (or composition or score) of one or more of the GAG forms (GAG properties) as described herein includes any measurable alteration or change of the GAG form (biomarker) (or score) in question when the GAG form in question is compared with a control level. An altered level (or score) includes an increased or decreased level (or score). Preferably, the level (or score) is significantly altered, compared to the level (or score or cut-off level) found in an appropriate control (e.g. control sample or subject or population). More preferably, the significantly altered levels or compositions or scores are statistically significant, preferably with a p-value of <0.05 or a % in ROPE value of ≤5.00.

In some embodiments, an alteration in level (or score) of ≥2%, ≥3%, ≥5%, ≥ 10%, ≥25%, ≥50%, ≥75%, ≥100%, ≥200%, ≥300%, ≥400%, ≥500%, ≥600%, ≥700%, ≥800%, ≥900%, ≥1000%, ≥2000%, ≥5000%, or ≥10,000% compared to the level (or score) found in an appropriate control sample or subject or population (i.e. when compared to a control level) may be indicative of the presence of cancer.

The “increase” in the level or “increased” level of one or more of the GAG forms (GAG properties) or scores as described herein includes any measurable increase or elevation of the GAG form (biomarker) (or score) in question when the GAG form (or score) in question is compared with a control level (or control score or cut-off level). Preferably, the level (or score) is significantly increased, compared to the level (or score or cut-off level) found in an appropriate control (e.g. control sample or subject or population). More preferably, the significantly increased levels (or scores) are statistically significant, preferably with a p-value of <0.05 or a % in ROPE value of ≤5.00.

In some embodiments, an increase in level (or score) of ≥2%, ≥3%, ≥5%, ≥ 10%, ≥25%, ≥50%, ≥75%, ≥100%, ≥200%, ≥300%, ≥400%, ≥500%, ≥600%, ≥700%, ≥800%, ≥900%, ≥1000%, ≥2000%, ≥5000%, or ≥10,000% compared to the level (or score) found in an appropriate control sample or subject or population (i.e. when compared to a control level or control score or cut-off level) may be indicative of the presence of cancer.

The “decrease” in the level or “decreased” level of one or more of the GAG forms (GAG properties) or scores as described herein includes any measurable decrease or reduction of the GAG form (biomarker) (or score) in question when the GAG form in question is compared with a control level (or control score or cut-off level). Preferably, the level (or score) is significantly decreased, compared to the level (or score or cut-off level) found in an appropriate control (e.g. control sample or subject or population). More preferably, the significantly decreased levels (or scores) are statistically significant, preferably with a p-value of <0.05 or a % in ROPE value of ≤5.00.

In some embodiments, a decrease in level (or score) of ≥2%, ≥3%, ≥5%, ≥ 10%, ≥25%, ≥50%, ≥75%, ≥100%, ≥200%, ≥300%, ≥400%, ≥500%, ≥600%, ≥700%, ≥800%, ≥900%, ≥1000%, ≥2000%, ≥5000%, or ≥10,000% compared to the level (or score) found in an appropriate control sample or subject or population (i.e. when compared to a control level or control score or cut-off level) is indicative of the presence of cancer.

A “control level” is the level of a GAG form (GAG property) in a control subject or population (e.g. in a sample that has been obtained from a control subject or population). Appropriate control subjects or samples for use in the methods of the invention would be readily identified by a person skilled in the art, for example an appropriate control group is as described in the Examples. Such subjects might also be referred to as “normal” subjects or as a reference population. Examples of appropriate populations of control subjects would include healthy subjects, for example, individuals who have no history of any form of cancer and no other concurrent disease. Other preferred control subjects would include individuals who are not suffering from, and preferably have no history of, cancer (e.g. not suffering from, and preferably have no history of, any of the types of cancer referred to herein). Where specific cancers are concerned, appropriate control subjects would include individuals who have no history of any form of disease, e.g. cancer, in the organ or tissue concerned for that particular cancer, and preferably no other concurrent disease. Preferably such control subjects are also not suffering from inflammatory pathologies. Preferably control subjects are not regular users of any medication. In a preferred embodiment control subjects are healthy subjects.

The control level may correspond to the level of the equivalent GAG form in appropriate control subjects or samples, e.g. may correspond to a cut-off or threshold level or range found in a control or reference population. Alternatively, said control level may correspond to the level of the marker (GAG form) in question in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements of GAG form(s) in individuals, either healthy or ill, are taken looking for changes in the levels of the GAG form(s). In this regard, an appropriate control level will be the individual's own baseline, stable, nil, previous or dry value (as appropriate) as opposed to a control or cut-off level found in the general control population. Control levels may also be referred to as “normal” levels or “reference” levels. The control level may be a discrete figure or a range.

Although the control level for comparison could be derived by testing an appropriate set of control subjects, the methods of the invention would not necessarily involve carrying out active tests on control subjects as part of the methods of the present invention but would generally involve a comparison with a control level which had been determined previously from control subjects and was known to the person carrying out the methods of the invention.

A “control chemical composition” is the chemical composition in a control subject or population (e.g. in a sample that has been obtained from a control subject or population). The discussion above in relation to a “control level” (e.g. appropriate control subjects, control samples, control populations, etc.) may be applied, mutatis mutandis, to “a control chemical composition”.

As described elsewhere herein, screening for cancer in accordance with the present invention may involve using a score (or a GAG score), or expressing the level and/or chemical composition using a score (or GAG score). In some such embodiments, an altered score (e.g. increased or decreased as the case may be) in comparison to a control score (or cut-off level or threshold level) is indicative of cancer in said subject. The discussion above in relation to a “control level” (e.g. appropriate control subjects, control samples, control populations, etc.) may be applied, mutatis mutandis, to “a control score”.

As described elsewhere herein, in some preferred methods of the present invention, the method comprises determining the level and/or chemical composition of the protein-free fraction of one or both of the GAGs chondroitin sulfate (CS) and heparin sulfate (HS) (or a score based thereon). In some such embodiments, an altered level and/or chemical composition of the protein-free fraction (or a score based thereon) in comparison to a level and/or chemical composition of the protein-free fraction of said one or both GAGs in a control (e.g. control sample or control score or cut-off level) is indicative of cancer. The discussion above in relation to a “control level” or “control chemical composition” or “control score” (e.g. appropriate control subjects, control samples, control populations, etc.) may be applied, mutatis mutandis, to embodiments of the invention that comprise determining the level and/or chemical composition of the protein-free fraction of one or both of said GAGs (or a score based thereon).

The methods of screening, diagnosis etc., of the present invention are for cancer. Some of the screening, etc., methods of the invention are generally applicable to a wide variety of cancers and can, if desired be used to screen, etc., for the presence or likelihood of cancer in a subject as opposed to any specific type of cancer. Once the methods of the invention have indicated the possible presence of cancer, follow up tests may be carried out, if desired, to identify the specific cancer that is present in the individual subject.

Also described herein are methods of screening, etc., for specific cancers.

The methods of the present invention can be carried out on any stage of cancer, for example can be used for early or initial stages of cancer or advanced or late stage cancer disease. Alternatively viewed, methods of the present invention can be carried out on any grade of cancer, for example can be used for low grade cancers, intermediate grade cancers or high grade cancers.

Thus, the methods of the present invention can be carried out for cancers selected from the group consisting of a stage I cancer, a stage II cancer, a stage III cancer, a stage IV cancer and a cancer of an unspecified stage.

In some embodiments, and advantageously, the methods of the invention can be used to screen for early stage or stage I cancer or low grade cancer. In some embodiments, the methods of the invention can be used to screen for a stage II cancer. In some embodiments, the methods of the invention can be used to screen for a stage III cancer. In some embodiments, the methods of the invention can be used to screen for a stage IV cancer or high grade cancer.

The classification of cancer at a given stage may be in accordance with any art recognised and accepted definition.

For example, depending on the type of cancer, a stage I or low grade cancer may be categorised as FIGO stage I, Ann Arbor stage 1, Binet stage A, Breast cancer grade 1-2, Low-grade (non glioblastoma) glioma, Gleason grade sum <7, TNM stage I or ENETS grade 1.

Depending on the type of cancer, a stage II cancer may be categorised as FIGO stage II, Ann Arbor stage 2, Binet stage B, or TNM stage II or IIA or IIB.

Depending on the type of cancer, a stage III cancer may be categorised as FIGO stage III, Ann Arbor stage 3, or TNM stage III or IIIA or IIIB or IIIC.

Depending on the type of cancer, a stage IV or high grade cancer may be categorised as FIGO stage IV, Ann Arbor stage 4, Binet stage C, Breast cancer grade 3, High-grade (glioblastoma) glioma, Gleason grade sum >=7, TNM stage IV or IVA or IVB or IVC, or ENETS grade 2.

The skilled person is familiar with which staging/grading systems or conventions are appropriate for which types of cancer and an appropriate staging/grading systems can be readily employed based on the type of cancer.

The classification of cancer at a given risk can be carried out by any art recognised and accepted definition. For example, risk may be assessed by determining the stage (e.g. using a staging system as set out above) of cancer at clinical diagnosis or by determining the stage (e.g. using a staging system as set out above) of a cancer at pathological diagnosis or by evaluating biopsy results (e.g. by evaluating tumor size, and/or tumor grade and/or tumor volume) or by assessing the results of other tests (x-rays, CT and/or MRI scans, or bone scans) or any combination of the above. A person skilled in the art can readily determine risk based on one or more of the assessments above. By way of an example, prostate cancer could be deemed low risk if the Gleason score is 6 or less, intermediate risk if the Gleason score is 7, and high risk if the Gleason score is 8 or more.

In some embodiments, the cancer may be a non-metastatic form of cancer. In some embodiments, the cancer may be a metastatic form of cancer (as opposed to localized or confined cancer).

The methods of the present invention can be carried out on any appropriate body fluid sample. In this regard, although the present invention is exemplified with blood (e.g. plasma) and urine, appropriate GAG forms to be measured in other types of body fluid sample could be determined by a skilled person following the teaching as provided herein. Typically the sample has been obtained from (removed from) a subject (e.g. as described elsewhere herein), preferably a human subject. In other aspects, the method further comprises a step of obtaining a sample from the subject.

Reference herein to “body fluid” includes reference to all fluids derived from the body of a subject. Exemplary fluids include blood (including all blood derived components, for example plasma, serum, etc.), urine, saliva, tears, bronchial secretions or mucus. Preferably, the body fluid is a circulatory fluid (especially blood or a blood component) or urine. Especially preferred body fluids are blood (e.g. plasma) or urine. Particularly preferred body fluids are plasma or urine. In some preferred embodiments the sample is a blood sample (e.g. a plasma or serum sample). In some preferred embodiments the sample is a plasma sample. In some embodiments a plasma sample is a platelet-poor plasma sample. In some preferred embodiments the sample is a serum sample. In some preferred embodiments the sample is a urine sample. In some embodiments, the sample is not a urine sample. In some embodiments, the sample is not a blood sample (e.g. not a plasma sample). The body fluid or sample may be in the form of a liquid biopsy.

The term “sample” also encompasses any material derived by processing a body fluid sample (e.g. derived by processing a blood (e.g. plasma) or urine sample). Processing of biological samples to obtain a test sample may involve one or more of: digestion, boiling, filtration, distillation, centrifugation, lyophilization, fractionation, extraction, concentration, dilution, purification, inactivation of interfering components, addition of reagents, derivatization, complexation and the like, e.g. as described elsewhere herein. Suitable processing steps can be selected depending on the features of the method being performed.

Any suitable method for isolating body fluid samples (e.g. urine or blood (e.g. serum or plasma) samples) may be employed.

Any sample that is directly or indirectly affected by the suspected cancer (e.g. tumour) may be used. Samples (e.g. original or unprocessed samples) typically comprise a protein-free fraction of GAGs and a protein-bound fraction of GAGs (as discussed elsewhere herein). In preferred methods of the present invention, the level and/or chemical composition of the protein-free fraction of one or both of the GAGs chondroitin sulfate (CS) and heparan sulfate (HS) is determined. Thus, in preferred embodiments a body fluid sample has been processed (or is processed) such that only (or essentially only) the protein-free fraction of said GAGs (typically disaccharide units derived therefrom) is subsequently analysed (i.e. processed such that the level and/or chemical composition of the protein-free fraction of said one or both GAGs (typically disaccharide units derived therefrom) is subsequently determined). In other methods of the invention, the sample has been processed (or is processed) such that the entire (protein-free plus protein-bound) fraction or pool of said one or both GAGs is subsequently analysed (i.e. processed such that the level and/or chemical composition of the entire (protein-free plus protein-bound) fraction or pool of said one or both GAGs (typically disaccharide units derived therefrom) is subsequently determined).

In some embodiments, a sample may comprise circulating tumour cells (e.g. metastatic tumour cells).

The term “sample” also encompasses any material derived by processing (e.g. as described above) a biological sample. Derived materials include disaccharide units (or a population of disaccharide units) derived by processing GAGs (e.g. as described elsewhere herein).

In some embodiments, methods of the invention may include a step of processing a sample. In some embodiments, methods of the invention may thus be performed on such processed samples or materials derived from such processed samples. In some embodiments, methods of the invention may thus be performed on samples that have been processed. Processing steps include, but are not limited to, isolating cells from the sample, isolating cell components from the sample, and extracting (e.g. isolating or purifying) proteins/peptides (although as preferred methods of the invention involve the determination of the protein-free GAG fraction, the extraction of proteins or the removal of the protein component from the proteoglycans (protein-bound GAGs) present in the sample, e.g. by digesting or otherwise removing the protein component is preferably not carried out). A processing step may involve one or more of filtration, distillation, centrifugation, extraction, concentration, dilution, purification, inactivation of interfering components, addition of reagents, derivatization, amplification, adapter ligation, and the like.

Samples can be used immediately or can be stored for later use (e.g. at −80° C.).

The methods of the invention as described herein can be carried out on any type of subject which is capable of suffering from cancer. The methods are generally carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs). Preferably the subject is a human. In the case of prostate cancer screening, typically the subject is a male mammal, e.g. a male human.

In one embodiment, the subject (e.g. a human) is a subject at risk of developing cancer or at risk of the occurrence of cancer, e.g. a healthy subject or a subject not displaying any symptoms of disease, or any other appropriate “at risk” subject as described elsewhere herein. In another embodiment the subject is a subject having, or suspected of having (or developing), or potentially having (or developing) cancer.

In some aspects, a method of the invention may further comprise an initial step of selecting a subject (e.g. a human subject) at risk of developing cancer or at risk of the occurrence of cancer, or having or suspected of having (or developing) cancer, or potentially having (or developing) cancer. The subsequent method steps can be performed on a sample from such a selected subject.

In some aspects, methods of the invention are provided which further comprise a step of treating cancer by therapy (e.g. pharmaceutical therapy) or surgery. For example, if the result of a method of the invention is indicative of cancer in the subject (e.g. a positive diagnosis of cancer is made), then an additional step of treating the cancer by therapy or surgery can be performed. For example, if the result of a method of the invention is indicative of high risk (or high grade) cancer in the subject (e.g. a positive diagnosis of high risk cancer is made), then an additional step of treating the cancer by therapy or surgery can be performed. For example, if the result of a method of the invention is indicative of low risk (or low grade) cancer in the subject (e.g. a positive diagnosis of low risk cancer is made), then an additional step of watchful waiting or active surveillance can be performed. Methods of treating cancer by therapy or surgery are known in the art. For example, one surgical option for prostate cancer is prostatectomy, which is aimed at eradication of the tumour and can be either radical (total removal) or partial. Pharmaceutical treatment can include standard chemotherapy and immunotherapy and hormone therapy, in addition to therapies which are subject to ongoing clinical trials. Pharmaceutical treatment can include standard chemotherapy (e.g. gemcitabine, vinblastine, floxuridine, 5-fluorouracil, or capecitabine), targeted therapies (including tyrosine kinase inhibitors, mTOR pathway inhibitors, VEGF pathway inhibitors, Janus kinase inhibitors, ALK inhibitors, Bcl-2 inhibitors, PARP inhibitors, PI3K inhibitors, Braf inhibitors, MEK inhibitors, CDK inhibitors, Hsp90 inhibitors or more specific examples such as imatinib, gefitinib, erlotinib, sorafenib, sunitinib, dasatinib, lapatinib, nilotinib, bortezomib, tamoxifen, tofacitinib, crizotinib, navitoclax, gossypol, iniparib, olaparib, perifosine, apatinib, zoptarelin doxorubicin (AN-152), doxorubicin linked to [D-Lys(6)]-LHRH, vemurafenib, dabrafenib, LGX818, trametinib, pazopanib, cabozantinib, axitinib, temsirolimus, everolimus, vemurafenib, dabrafenib, lenvatinib), immunotherapy (such as interferon-gamma, interleukin-2, interferon-alpha, or PD-1 or PD-L1 blockers such as pembrolizumab, ipilimumab, nivolumab), other monoclonal antibody therapies (such as rituximab, trastuzumab, alemtuzumab, cetuximab, panitumumab, bevacizumab), and hormone treatment (such as orchiectomy, luteinizing hormone-releasing hormone agonists or antagonists, anti-androgens, estrogen, or ketoconazole). Other forms of treatment include radiation therapy and cryotherapy and vaccine treatment (such as chimeric antigen receptors engineered (CAR) T cells).

Thus, in some embodiments, methods of the invention (e.g. screening or diagnosis methods) which further comprise a step of treating cancer may comprise administering to the subject a therapeutically effective amount of one or more agents selected from the group consisting of a chemotherapeutic agent, for example selected from gemcitabine, vinblastine, floxuridine, 5-fluorouracil, or capecitabine; an agent for targeted therapy, for example selected from tyrosine kinase inhibitors, mTOR pathway inhibitors, VEGF pathway inhibitors, Janus kinase inhibitors, ALK inhibitors, Bcl-2 inhibitors, PARP inhibitors, PI3K inhibitors, Braf inhibitors, MEK inhibitors, CDK inhibitors, Hsp90 inhibitors or more specific examples such as imatinib, gefitinib, erlotinib, sorafenib, sunitinib, dasatinib, lapatinib, nilotinib, bortezomib, tamoxifen, tofacitinib, crizotinib, navitoclax, gossypol, iniparib, olaparib, perifosine, apatinib, zoptarelin doxorubicin (AN-152), doxorubicin linked to [D-Lys(6)]-LHRH, vemurafenib, dabrafenib, LGX818, trametinib, pazopanib, cabozantinib, axitinib, temsirolimus, everolimus, vemurafenib, dabrafenib, lenvatinib; or an agent for immunotherapy, for example selected from interferon-gamma, interleukin-2, interferon-alpha, or PD-1 or PD-L1 blockers such as pembrolizumab, ipilimumab, nivolumab, or an agent for hormone treatment, for example selected from luteinizing hormone-releasing hormone agonists (for example leuprolide, goserelin, triptorelin, histrelin) or antagonists (for example degarelix or CYP17 inhibitors), anti-androgens (for example flutamide, bicalutamide, nilutamide) or estrogen or ketoconazole.

Alternatively, methods of the invention are provided which further comprise a step of carrying out an additional diagnostic procedure, e.g. a CT scan (or a PSA test in the case of prostate cancer).

Thus, in some embodiments, methods of the invention (e.g. screening or diagnosis methods) which further comprise a step of treating cancer may comprise administering to the subject a therapeutically effective amount of one or more agents selected from the group consisting of a chemotherapeutic agent, an agent for targeted therapy, or an agent for immunotherapy, or an agent for hormone therapy, or a dose of radiation therapy, or a dose of cryotherapy.

In some embodiments, if the level of one or more GAG properties in a sample, or a score based on these levels, is altered by a particular degree in comparison to a control level or score (or cut-off level), then a further step of administering a therapeutically effective amount of a pharmaceutical agent (e.g. a chemotherapeutic agent etc., as described above) to the patient is performed and/or surgery is performed. Preferred degrees of alteration are discussed elsewhere herein.

In some embodiments, if a subject is already undergoing pharmaceutical therapy (e.g. chemotherapeutic therapy or other therapy as described above) and the level of one or more GAG properties in a sample, or a score based on these levels, is altered (or indeed not altered) by a particular degree in comparison to a control level (e.g. in comparison to a previously recorded level or score for the same subject), then this may be indicative that the current therapeutic agent is not being effective and that a therapeutic agent other than the previous therapeutic agent should be used. Thus, a step of administering a therapeutically effective amount of a therapeutic agent (e.g. a chemotherapeutic agent etc. as described above) other than the therapeutic agent previously administered to the subject may be performed.

In some embodiments, if a method of the invention reveals that a current treatment regimen is ineffective, e.g. if serial or periodic measurements of one or more GAG properties in a sample, or a score based on these levels, reveal treatment is being ineffective, a step of altering (e.g. increasing) the dosage of the therapeutic agent may be performed.

More specifically for example, in some such aspects of the invention, methods are provided which comprise determining the level of one or more GAG properties in a sample and if one or more levels, or a score based on these levels, are determined to be greater than an appropriate cut-off level, e.g. a cut-off level pre-specified to maximise the accuracy for a positive diagnosis of cancer, then said methods may comprise the further step of performing a surgery or performing an additional diagnostic procedure (e.g. a CT scan), or administering a therapeutically-effective amount of a recommended drug agent for the treatment of cancer. Agents for example may comprise standard chemotherapy (e.g. gemcitabine, vinblastine, floxuridine, 5-fluorouracil, or capecitabine), targeted therapies (including tyrosine kinase inhibitors, mTOR pathway inhibitors, VEGF pathway inhibitors, Janus kinase inhibitors, ALK inhibitors, Bcl-2 inhibitors, PARP inhibitors, PI3K inhibitors, Braf inhibitors, MEK inhibitors, CDK inhibitors, Hsp90 inhibitors or more specific examples such as imatinib, gefitinib, erlotinib, sorafenib, sunitinib, dasatinib, lapatinib, nilotinib, bortezomib, tamoxifen, tofacitinib, crizotinib, navitoclax, gossypol, iniparib, olaparib, perifosine, apatinib, zoptarelin doxorubicin (AN-152), doxorubicin linked to [D-Lys(6)]-LHRH, vemurafenib, dabrafenib, LGX818, trametinib, pazopanib, cabozantinib, axitinib, temsirolimus, everolimus, vemurafenib, dabrafenib, lenvatinib), immunotherapy (such as interferon-gamma, interleukin-2, interferon-alpha, or PD-1 or PD-L1 blockers such as pembrolizumab, ipilimumab, nivolumab), other monoclonal antibody therapies (such as rituximab, trastuzumab, alemtuzumab, cetuximab, panitumumab, bevacizumab), or hormone therapies, for example luteinizing hormone-releasing hormone agonists (for example leuprolide, goserelin, triptorelin, histrelin) or antagonists (for example degarelix or CYP17 inhibitors), anti-androgens (for example flutamide, bicalutamide, nilutamide) or estrogen or ketoconazole.

Conversely, methods are provided which comprise determining the level of one or more GAG properties in a sample and if one or more levels, or a score based on these levels, are determined to be lower than an appropriate cut-off level, e.g. a cut-off level pre-specified to maximise the predictive value for a negative diagnosis of cancer, then said methods may comprise the further step of not performing a surgery, or performing an additional diagnostic procedure (e.g. a CT scan) or altering the current dosage of drug agent(s) or administering a therapeutically-effective amount of a distinct recommended drug agent for cancer (e.g. prostate cancer) to the one already being used. Agents for example may comprise standard chemotherapy (e.g. gemcitabine, vinblastine, floxuridine, 5-fluorouracil, or capecitabine), targeted therapies (including tyrosine kinase inhibitors, mTOR pathway inhibitors, VEGF pathway inhibitors, Janus kinase inhibitors, ALK inhibitors, Bcl-2 inhibitors, PARP inhibitors, PI3K inhibitors, Braf inhibitors, MEK inhibitors, CDK inhibitors, Hsp90 inhibitors or more specific examples such as imatinib, gefitinib, erlotinib, sorafenib, sunitinib, dasatinib, lapatinib, nilotinib, bortezomib, tamoxifen, tofacitinib, crizotinib, navitoclax, gossypol, iniparib, olaparib, perifosine, apatinib, zoptarelin doxorubicin (AN-152), doxorubicin linked to [D-Lys(6)]-LHRH, vemurafenib, dabrafenib, LGX818, trametinib, pazopanib, cabozantinib, axitinib, temsirolimus, everolimus, vemurafenib, dabrafenib, lenvatinib), immunotherapy (such as interferon-gamma, interleukin-2, interferon-alpha, or PD-1 or PD-L1 blockers such as pembrolizumab, ipilimumab, nivolumab), other monoclonal antibody therapies (such as rituximab, trastuzumab, alemtuzumab, cetuximab, panitumumab, bevacizumab), or hormone therapies, for example luteinizing hormone-releasing hormone agonists (for example leuprolide, goserelin, triptorelin, histrelin) or antagonists (for example degarelix or CYP17 inhibitors), anti-androgens (for example flutamide, bicalutamide, nilutamide) or estrogen or ketoconazole.

A yet further aspect provides a kit for screening for cancer (e.g. for diagnosing or for determining severity or prognosis of cancer), which comprises one or more agents suitable for determining the level of one or more of the GAG properties (GAG forms) described herein, in a sample. A yet further aspect provides a kit for screening for cancer (e.g. for diagnosing or for determining severity or prognosis of cancer), which comprises one or more reagents (or components) for processing a body fluid sample that comprises GAGs whose level and/or chemical composition is determined in accordance with the invention. In preferred aspects said kits are for use in the methods of the invention as described herein. Preferably said kits comprise instructions for use of the kit components, for example in screening (e.g. diagnosis).

In one aspect, the present invention provides a method of detecting (or determining) the level and/or chemical composition of the protein-free fraction of one or both of the glycosaminoglycans (GAGs) chondroitin sulfate (CS) and heparan sulfate (HS) in a body fluid sample, wherein said sample has been obtained from said subject.

In one aspect, the present invention provides a method of detecting (or determining) the level and/or chemical composition of the protein-free fraction of one or both of the glycosaminoglycans (GAGs) chondroitin sulfate (CS) and heparan sulfate (HS), said method comprising:
(a) obtaining a body fluid sample from a human patient; and
(b) detecting (or determining) the level and/or chemical composition of the protein-free fraction one or both of the glycosaminoglycans (GAGs) chondroitin sulfate (CS) and heparan sulfate (HS) in said sample.

A yet further aspect of the invention provides a method of detecting (or determining) the level and/or chemical composition of one or both of the glycosaminoglycans (GAGs) chondroitin sulfate (CS) and heparan sulfate (HS) in a body fluid sample,
wherein said sample has been obtained from said subject and has been subjected to processing prior to determining said level and/or chemical composition,
   wherein said processing
      (a) comprises fragmenting said one or both GAGs into disaccharide units; and
      (b) does not comprise prior to (a) at least one of:
         (i) contacting said sample with a proteolytic agent; and
         (ii) purifying said one or both GAGs in said sample based on the negative charge of said GAGs.

The features and discussion herein in relation to the method of screening for cancer (e.g. method of diagnosing, method for prognosis etc.), for example in relation to preferred GAG forms or combinations thereof for measurement, can be applied, mutatis mutandis, to methods of detecting of the present invention.

Where the terms “comprise”, “comprises”, “comprising”, “has” or “having”, or other equivalent terms are used herein, then in some more specific embodiments these terms include the term “consists of” or “consists essentially of”, or other equivalent terms.

The invention will be further described with reference to the following non-limiting Example with reference to the following drawings in which:

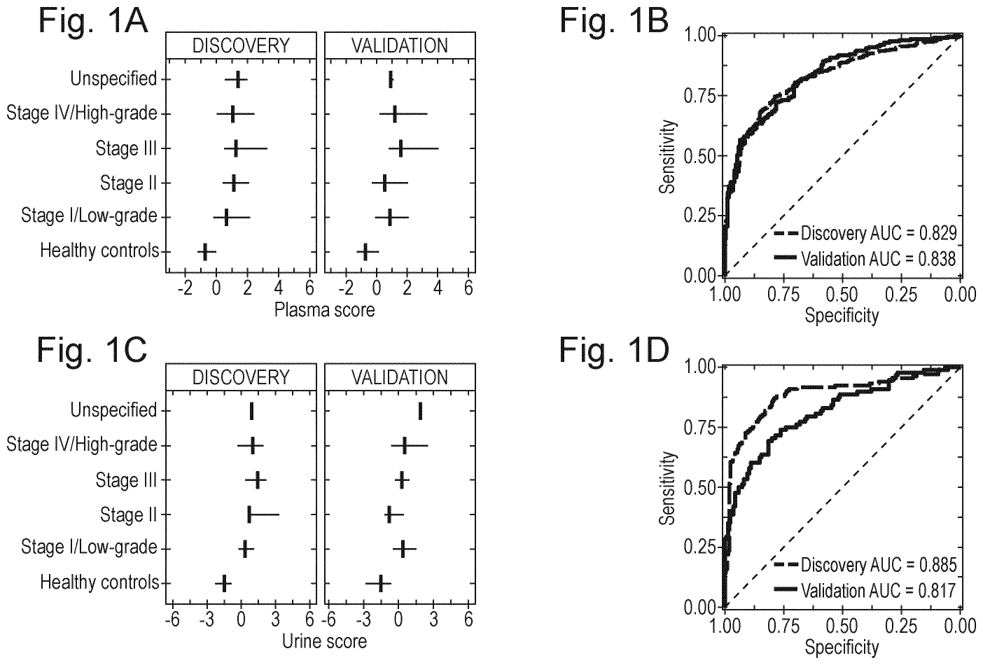

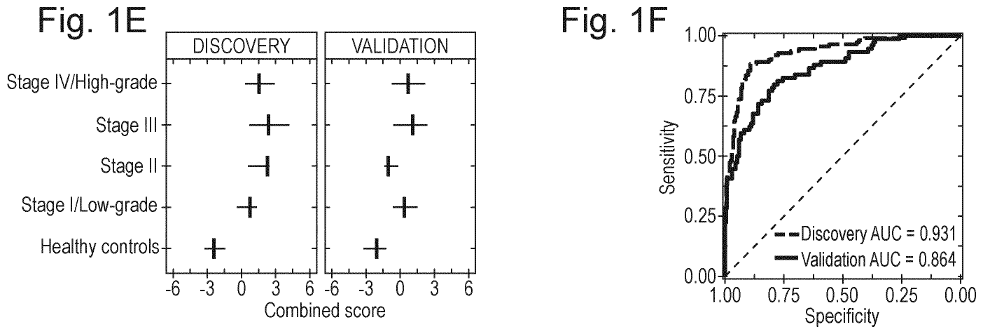

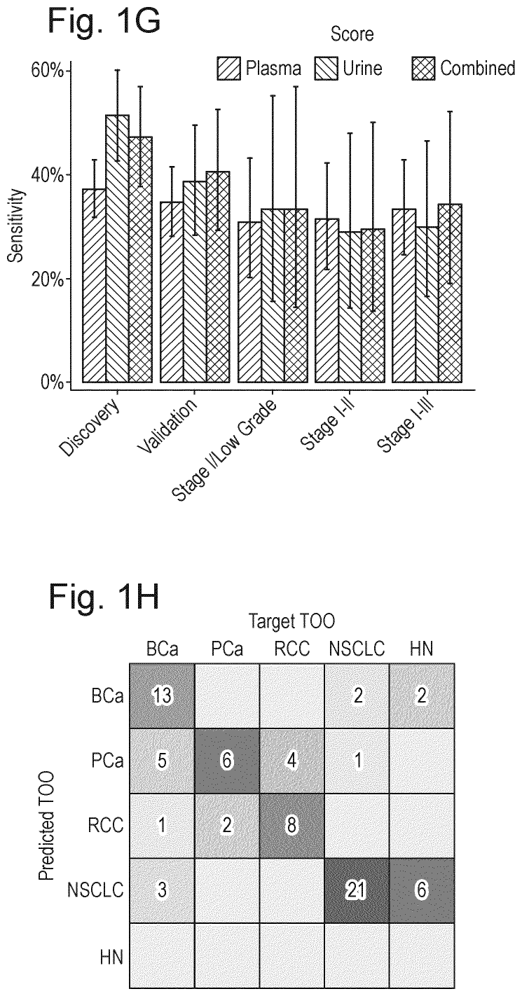

FIG. 1: A) Plasma pan-cancer GAG scores across different stage/grade groups and B) ROC curve in the discovery (60% samples) and validation sets (40% samples) for the plasma pan-cancer GAG score. C)-D) as A) and B) for the urine pan-cancer GAG score. E)-F) as A) and B) for the combined pan-cancer GAG score. G) Sensitivity at 98% specificity for the plasma, urine, and combined pan-cancer GAG scores in the discovery and validation sets and across different stage/grade groups. H) Tissue of origin (TOO) prediction in the validation set (N=74, 5 cancer types) using a Bayesian Additive Regression Trees model trained on combined GAGomes in the discovery set (N=110). Key: see Table 1.

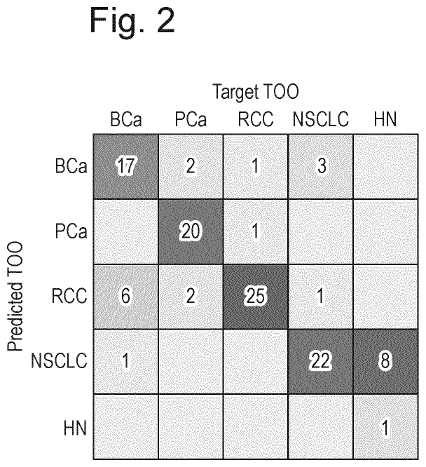

FIG. 2: Performance of the tissue-of-origin (TOO) classifier in the discovery cohort. The numbers in the boxes represent the number of samples classified as belonging to the predicted TOO. Overall and balanced accuracy at predicting the cancer's tissue of origin were 77.3% and 81.3%, respectively. N=110. Key—Table 1

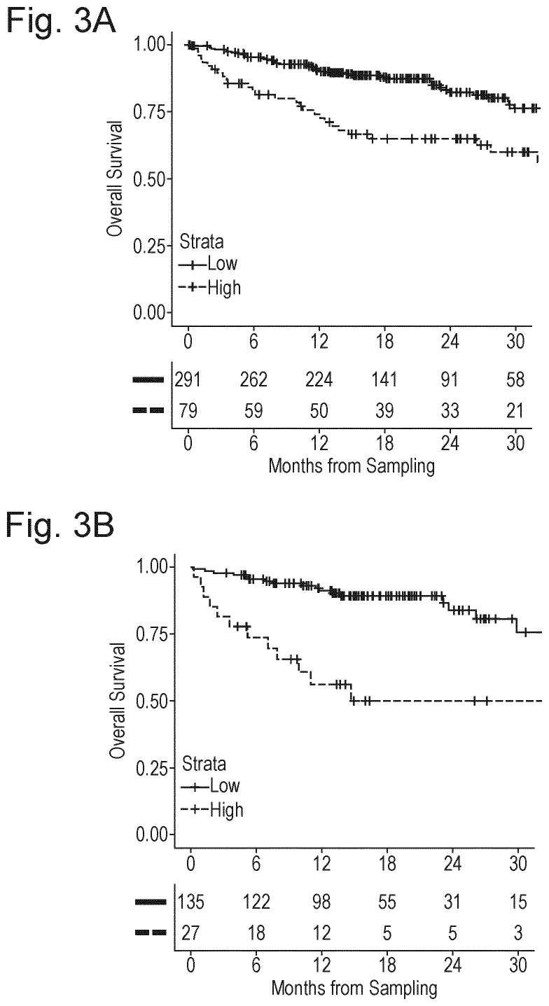

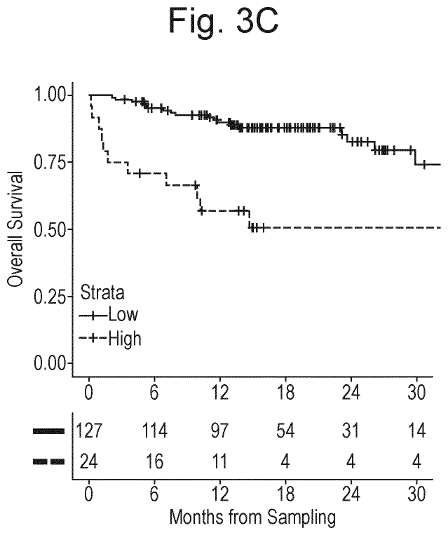

FIG. 3: Kaplan-Meier curves for overall survival in cancer patients stratified by GAG score values into ‘high’ versus ‘low’ risk. A) Plasma score. B) Urine score. C) Combined score.

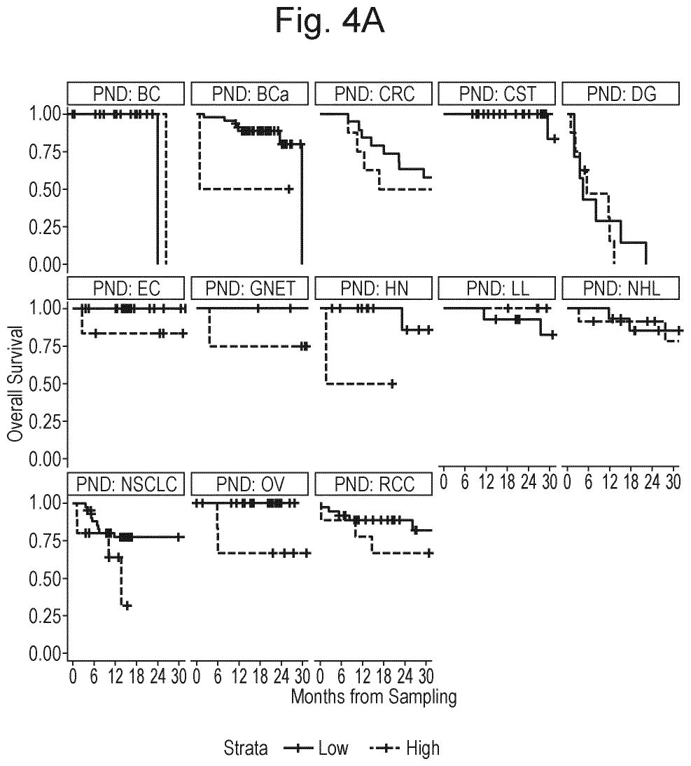

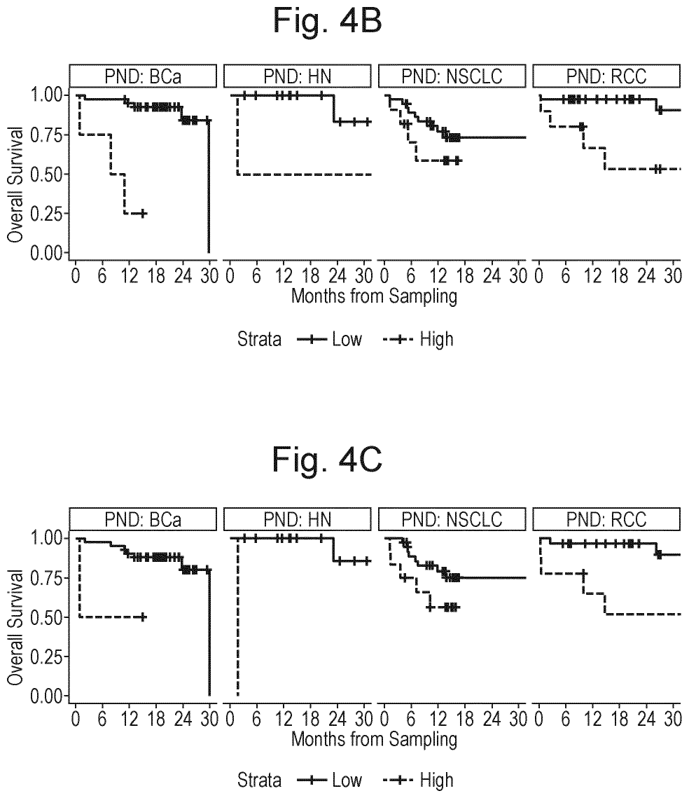

FIG. 4: Kaplan-Meier survival curves for overall survival for different cancer types, where patients are stratified into ‘high’ versus ‘low’ risk based on the A) plasma, B) urine, and C) combined GAG score above/below an optimal cut-off. Key: BC—Breast cancer; BCa—Bladder cancer; CRC—Colorectal cancer; CST—Cervical cancer; DG—Diffuse Glioma; EC—Endometrial Carcinoma; GNET—Small Intestinal Neuroendocrine Tumor; HN—Head and Neck Cancer; LL—B-cell Leukemia; NHL—Non-follicular Lymphoma; NSCLC—Non-small-cell Lung Carcinoma; OC—Ovarian Carcinoma; PCa—Prostate cancer; RCC—Renal Cell Carcinoma.

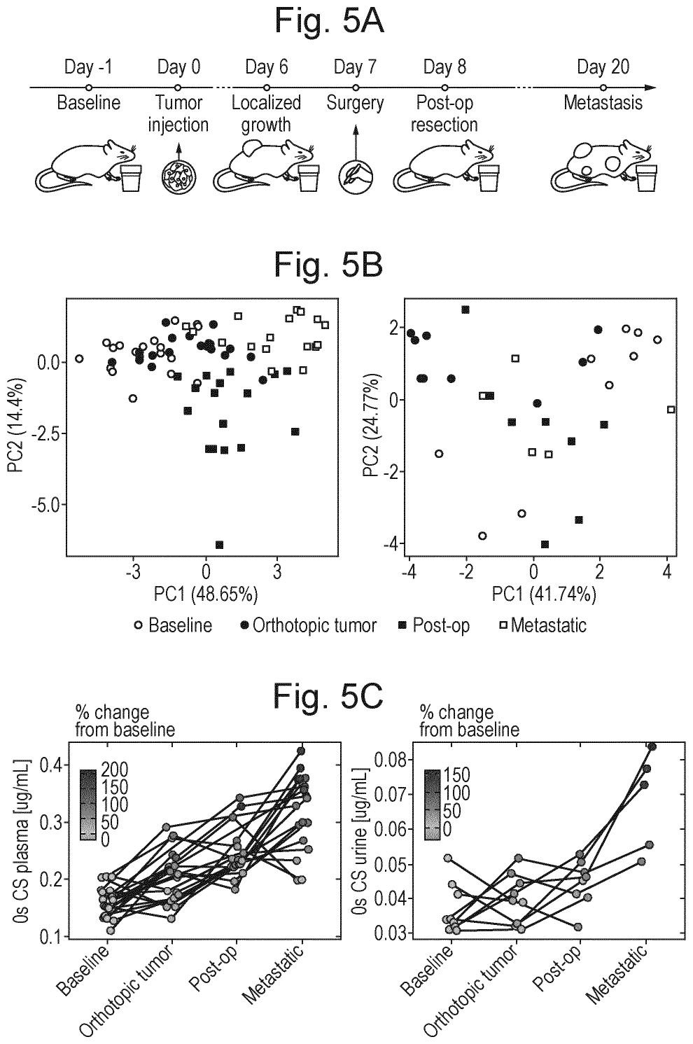

FIG. 5: Alterations of biofluidic glycosaminoglycans during cancer progression in mice. A) Experimental design. B) Principal component analysis of plasma (left-hand panel) and urine (right-hand panel) GAGomes at different time-points of cancer progression. C) Longitudinal level of plasma and urine 0s CS, respectively, per individual mouse at different time-points of cancer progression.

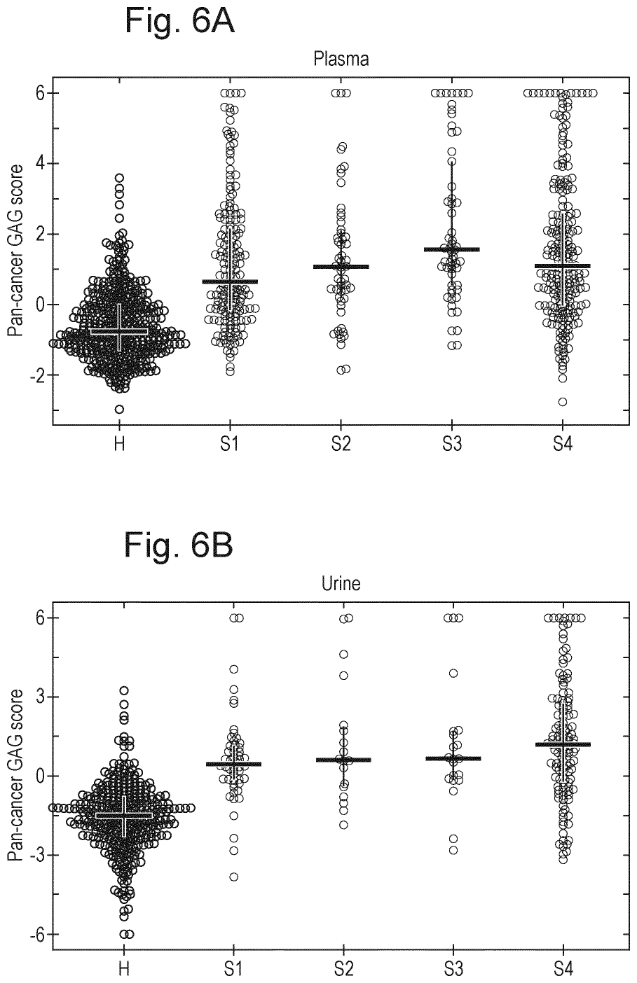

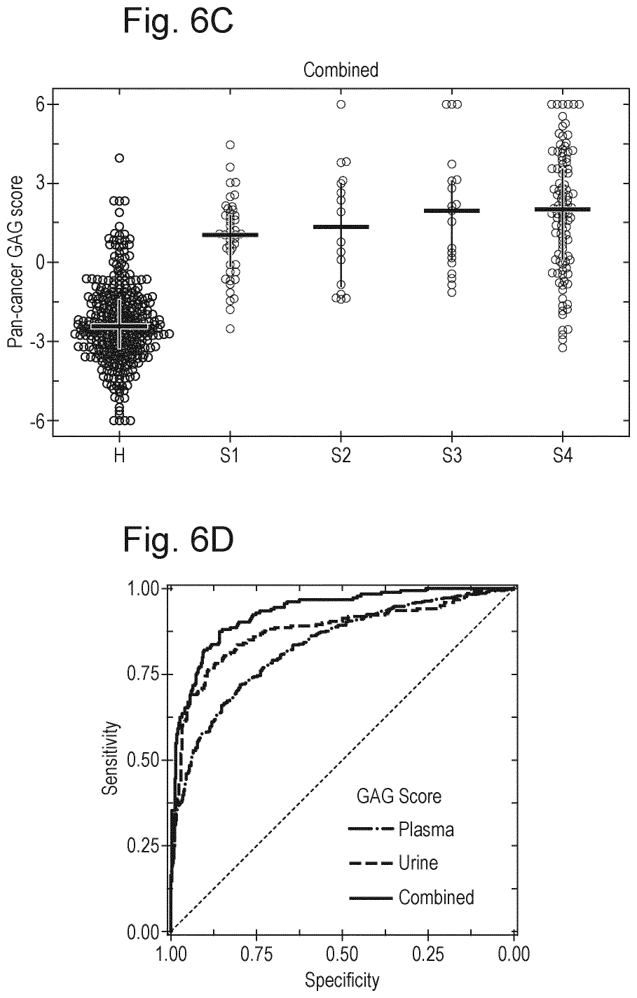

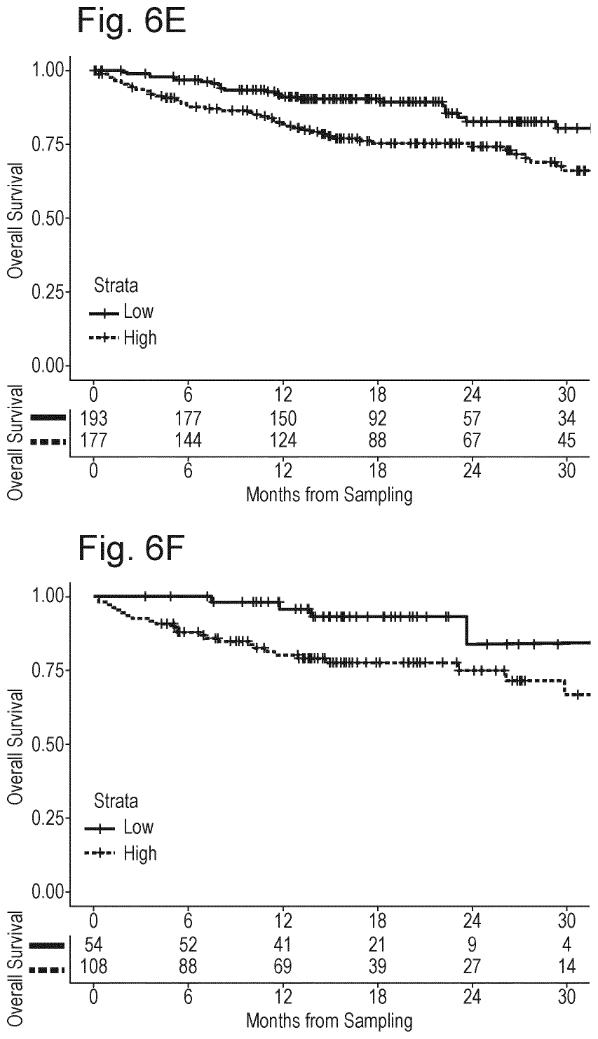

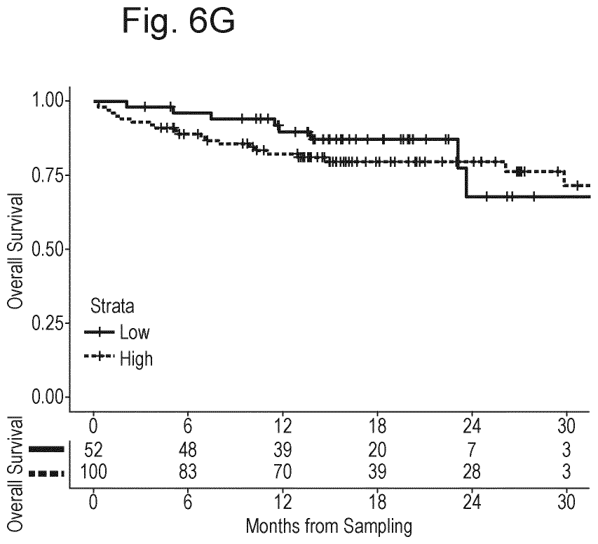

FIG. 6: Plasma, urine, and combined pan-cancer GAG scores of Example 2. A, B, C) Plasma, urine and combined pan-cancer GAG scores across different stage/grade groups, respectively. The crossbar denotes median and 25th/75th quantiles. D) ROC curves for plasma, urine and combined GAG scores. E, F, G) Kaplan-Meier curves for overall survival across all cancer patients stratified into groups of “Low” (undetected) vs. “High” (detected) pan cancer plasma (E), urine (F), and combined (G) GAG score values, respectively. For each score, the patients with scores higher than the cut-off at 95% specificity were assigned to the “High”, vs. “Low” group. The panels show number at risk for each group. Plasma pan-cancer GAG score (cut-off score=1.259, HR=0.61 [95% CI=0.44-0.85], p=0.0031. N=370, 13 cancer types), Urine (cut-off score=0.255, HR=0.50 [95% CI=0.25-0.98], p=0.0441. N=162, 4 cancer types). Combined (cut-off score=0.662, HR=0.86 [95% CI=0.50-1.49], p=0.595). N=152, 4 cancer types). Key: S1=Stage I/low grade, S2=Stage II, S3=Stage III, S4=Stage IV/High grade, H=healthy.

### EXAMPLE 1

### Introduction

We investigated urine and/or plasma glycosaminoglycans (GAGs) profiles—or GAGomes—comprising the groups of chondroitin sulfate (CS), heparan sulfate (HS), and hyaluronic acid (HA) disaccharides as biomarkers of cancer metabolism for early detection of 14 types in a total of 979 urine and/or plasma cancer vs. healthy subjects. In this study, the GAGomes of the protein-free fraction of plasma and urine samples was studied. We observed widespread cancer-specific changes in biofluidic GAGomes—recapitulated in an in vivo model of cancer progression. We developed machine learning models that detected any-stage, any-type cancer with a sensitivity up to 40.5% at 98% specificity, predicted tissue-of-origin with 74% accuracy, and independently correlated with overall survival. Overall, GAGomes were powerful and versatile metabolic biomarkers for cancer representing a new avenue for liquid biopsies.

### Study Design and GAGome Measurement

### Study Design and Patient Recruitment

The design was a case-control study with mixed retrospective and prospective cohorts. The study population comprised cases defined as patients with confirmed cancer diagnosis (no history of cancer, active disease [treatment naïve or metastatic disease]) across 14 cancer types) and controls defined as self-rated healthy subjects (moderated to very good health, no history nor known family history of cancer) and cancer-free patients initially suspected of colorectal cancer.

The retrospective cohorts with plasma samples were obtained from patients with breast ductal invasive carcinoma (BC), colorectal carcinoma (CRC), cervical squamous cell carcinoma (CST), diffuse glioma (DG), small intestinal neuroendocrine tumor (GNET), endometrial adenocarcinoma (EC), chronic lymphoid leukaemia (LL), diffuse large B-cell lymphoma (NHL), non-small cell lung cancer (NSCLC), ovarian epithelial carcinoma (OV) and prostate adenocarcinoma (PCa) as well cancer-free patients initially suspected for CRC as an additional control group. The inclusion criteria were: for cases—diagnosis of cancer with any of the above-mentioned cancer types; active disease [treatment naïve or metastatic disease] at the time of sampling; older than 18 year old at the time of sampling; for controls—patients initially suspected for CRC and cancer-free at final diagnosis; older than 18 year old at the time of sampling. The exclusion criteria were: history of non-basal cell carcinoma cancer; samples collected before 2015 for all diagnosis groups except CRC (2012) and LL (2014). Patients were retrospectively identified by random choice based on the eligibility criteria so that between 14 to 40 patient per cancer type were included, of which 50% were early-stage/low-grade and 50% late-stage/high-grade (as defined below).

The prospective cohorts with plasma and urine samples were obtained from patients with bladder cancer (BCa), NSCLC, head and neck squamous cell carcinoma (HN), renal cell carcinoma (RCC), PCa and healthy controls. The eligibility criteria were: for NSCLC and HN—inclusion: ECOG performance status 0-2, diagnosis of non-small cell lung cancer or head and neck squamous cell carcinoma, metastatic disease, predicted life expectancy over 2 months, informed consent; and exclusion: lack of proper compliance to accept samplings; for PCa—inclusion: referral to robotic-assisted laparoscopic prostatectomy for prostate cancer, serum PSA at clinical diagnosis available, transrectal ultrasound guided biopsy report available, fit to undergo all protocol procedures and informed consent; no exclusion criteria; for RCC—inclusion: referral for partial or radical nephrectomy for suspicion of renal cell carcinoma, predicted life expectancy over 2 months, standard imaging evaluation 12 weeks prior to inclusion, planned for standard imaging within 16 weeks after start of therapy, and informed consent; exclusion: lack of proper compliance to accept continuous samplings; for the BCa—inclusion: referral to transurethral resection of the bladder (TURB) for BCa, informed consent, urine cytology available at diagnosis, fit to undergo all protocol procedures; exclusion: age less than 18 years, history of bladder or prostate radiation, prior diagnosis of cancer; for healthy controls—inclusion: self-rated health at least “moderate”, age between 21 and 78 years old, informed consent, fit to undergo all protocol procedures; exclusion: previous history of cancer (except non-melanoma skin cancer), family history of cancer among first-degree relatives, and for men, total serum PSA≥0.5 ng/ml within the last 5 years or upon registration.

Clinical data related to age, gender, eligibility criteria as well as date of death or last known alive, diagnosis and tumor grade or stage for all cases, was retrieved from patients' journals in the case arm and through a questionnaire in the healthy control arm. Cases were grouped by cancer type and by stage/grade. Specifically, we classified cases as early-stage/low-grade vs. stage IV/high-grade as follows: TNM I-III vs TNM IV in BC, CRC, NSCLC, RCC, BCa, HN; G1 (Mitotic count (10 HPF)<2 and Ki67<2) vs. G2 (Mitotic count (10 HPF) 2 to 20 and Ki67 3 to 20) in GNET; lower-grade glioma vs. glioblastoma multiforme in DG; FIGO stage I vs. II-IV in CST and I-II vs. III-IV in EC/OV; Binet stage A-B vs. C in LL; Anna Arbor stage I-II vs. III-IV in NHL; pathological Gleason grade <7 vs. >=7 in PCa. Further subsets were: stage I/low-grade including all early-stage/low-grade except TNM II-III, FIGO stage II, Binet stage B, and Ann Arbor stage II; stage II including TNM II, FIGO stage II, Binet stage B, and Ann Arbor stage II; stage Ill including TNM III, FIGO stage III, and Ann Arbor stage III. All participants provided informed consent at the recruitment sites under Ethical Committee approved protocols.

We conducted a case-control study including a total of 553 cancer patients across 14 types (N=14 to 104, median per type=28) and 426 healthy subjects with similar demographics characteristics across multi-site international cohorts. Thirty-four percent of all cancer patients were classified as stage I/low-grade (6% to 66% across types, median per type=41%). Patient characteristics are summarized in Table 1.

TABLE 1
Study population characteristics

| | | | Stage I/ | | | | | | Stage IV/ | | Unspecified | |
|---|---|---|---|---|---|---|---|---|---|---|---|---|
| | Healthy | | Low-grade | | Stage II | | Stage III | | High-grade | | stage/grade | |
| | controls | | (N = 187, | | (N = 56, | | (N = 59, | | (N = 238, | | (N = 13, | |
| | (N = 426) | | 33.8%) | | 10.1%) | | 10.7%) | | 43%) | | 2.4%) | |
| Age | | | | | | | | | | | | |
| Mean (SD) | 57.4 | (13.8) | 61.7 | (15.3) | 62.5 | (13.1) | 63.7 | (14.7) | 66.6 | (8.81) | 68.0 | (9.71) |
| Median [Min, Max] | 59.0 | [22.0, 78.0] | 64.0 | [22.0, 91.0] | 66.5 | [36.0, 84.0] | 67.0 | [21.0, 87.0] | 67.0 | [27.0, 89.0] | 69.0 | [49.0, 82.0] |
| Gender | | | | | | | | | | | | |
| Female | 246 | (57.7%) | 105 | (56.1%) | 31 | (55.4%) | 36 | (61.0%) | 77 | (32.4%) | 4 | (30.8%) |
| Male | 180 | (42.3%) | 82 | (43.9%) | 25 | (44.6%) | 23 | (39.0%) | 161 | (67.6%) | 9 | (69.2%) |
| Sample availability | | | | | | | | | | | | |
| Blood | 86 | (20.2%) | 134 | (71.7%) | 38 | (67.9%) | 38 | (64.4%) | 112 | (47.1%) | 11 | (84.6%) |
| Blood and urine | 339 | (79.6%) | 44 | (23.5%) | 16 | (28.6%) | 19 | (32.2%) | 105 | (44.1%) | 0 | (0%) |
| Urine | 1 | (0.2%) | 9 | (4.8%) | 2 | (3.6%) | 2 | (3.4%) | 21 | (8.8%) | 2 | (15.4%) |
| Group | | | | | | | | | | | | |
| Healthy (H) | 426 | (100%) | | | | | | | | | | |
| Cancer (C) | 553 | | | | | | | | | | | |
| Breast cancer (BC) | | | 15 | (8.0%) | 10 | (17.9%) | 2 | (3.4%) | 1 | (0.4%) | 0 | (0%) |
| Bladder cancer | | | 31 | (16.6%) | 10 | (17.9%) | 6 | (10.2%) | 0 | (0%) | 0 | (0%) |
| (BCa) | | | | | | | | | | | | |
| Colorectal cancer | | | 2 | (1.1%) | 5 | (8.9%) | 7 | (11.9%) | 9 | (3.8%) | 4 | (30.8%) |
| (CRC) | | | | | | | | | | | | |
| Cervical cancer | | | 16 | (8.6%) | 9 | (16.1%) | 1 | (1.7%) | 2 | (0.8%) | 0 | (0%) |
| (CST) | | | | | | | | | | | | |
| Diffuse Glioma | | | 17 | (9.1%) | 0 | (0%) | 0 | (0%) | 23 | (9.7%) | 0 | (0%) |
| (DG) | | | | | | | | | | | | |
| Endometrial | | | 20 | (10.7%) | 3 | (5.4%) | 5 | (8.5%) | 2 | (0.8%) | 0 | (0%) |
| Carcinoma (EC) | | | | | | | | | | | | |
| Small Intestinal | | | 6 | (3.2%) | 0 | (0%) | 0 | (0%) | 8 | (3.4%) | 0 | (0%) |
| Neuroendocrine | | | | | | | | | | | | |
| Tumor (GNET) | | | | | | | | | | | | |
| Head and Neck | | | 1 | (0.5%) | 3 | (5.4%) | 1 | (1.7%) | 12 | (5.0%) | 0 | (0%) |
| Cancer (HN) | | | | | | | | | | | | |
| B-cell Leukemia | | | 11 | (5.9%) | 3 | (5.4%) | 0 | (0%) | 4 | (1.7%) | 0 | (0%) |
| (LL) | | | | | | | | | | | | |
| Non-follicular | | | 8 | (4.3%) | 6 | (10.7%) | 6 | (10.2%) | 7 | (2.9%) | 3 | (23.1%) |
| Lymphoma (NHL) | | | | | | | | | | | | |
| Non-small-cell Lung | | | 14 | (7.5%) | 0 | (0%) | 1 | (1.7%) | 67 | (28.2%) | 1 | (7.7%) |
| Carcinoma | | | | | | | | | | | | |
| (NSCLC) | | | | | | | | | | | | |
| Ovarian Carcinoma | | | 12 | (6.4%) | 1 | (1.8%) | 13 | (22.0%) | 4 | (1.7%) | 0 | (0%) |
| (OV) | | | | | | | | | | | | |
| Prostate cancer | | | 12 | (6.4%) | 0 | (0%) | 0 | (0%) | 87 | (36.6%) | 5 | (38.5%) |
| (PCa) | | | | | | | | | | | | |
| Renal Cell | | | 22 | (11.8%) | 6 | (10.7%) | 17 | (28.8%) | 12 | (5.0%) | 0 | (0%) |
| Carcinoma (RCC) | | | | | | | | | | | | |

We measured the plasma and urine GAGomes using a standardized UHPLC-MS/MS method (D. Tamburro, S. Bratulic, S. A. Shameh, N. K. Soni, A. Bacconi, F. Maccari, F. Galeotti, K. Mattsson, N. Volpi, J. Nielsen, F. Gatto, bioRxiv, in press, doi: 10.1101/2021.02.04.429694) in a single blinded laboratory. For a given fluid, the GAGome comprised the concentration of all disaccharide forms of chondroitin sulfate (CS), heparan sulfate (HS) and hyaluronic acid (HA) plus dependent properties such as CS and HS charge and CS and HS total concentration—for a total of 39 features. Details of the sample collection, processing and GAG measurements are set out below.

### Sample Collection

Across all cohorts, we successfully analyzed a total of 942 plasma samples and 560 urine, so divided: for the case arm, 517 plasma samples in 14 cancer types and 220 urine samples in 5 cancer types; and for the control arm 425 plasma and 340 urine samples. A subset of 184 cases (5 cancer types) and 339 healthy controls had matched plasma and urine samples. All samples were de-identified and registered according to applicable national law for bio-banking.

Whole blood samples were collected in K2 EDTA-coated tubes at room temperature and processed within 15 minutes. In general, the tubes were centrifuged (2500 RCF for 15 minutes at 4° C.) and the plasma was extracted and collected in a separate cryovial for storage at −80° C. until shipment in dry ice. In the healthy control cohort, plasma centrifugation was at 1100-1300 RCF at room temperature for 10 to 20 minutes. In the retrospective cohorts, plasma centrifugation was at 2400 RCF at room temperature for 7 minutes. In the BCa cohort, plasma centrifugation was at 2000 RCF at room temperature for 10 minutes. Urine was an any-void spot collection in polypropylene cups and 100-220 μL aliquoted into cryovials for storage at −20° C. until shipment in dry ice. In the BCa cohort, urine was also centrifuged at 2,000 RCF at room temperature for 5 minutes. Differences in sample collection are attributable to different protocols in the robotic collection of samples across sites and are not expected to exert a remarkable effect on GAG measurements (data not shown).

### Sample Processing and GAG Measurements and Analysis

The urine and plasma samples were processed to obtain GAG disaccharides for analysis.

The processing of the GAGs prior to ultra-high-performance liquid chromatography coupled with triple-quadrupole mass spectrometry was performed following Elypta MIRAM™ Glycosaminoglycan Kit instructions for use. All reagents and consumables used were contained in the kit. This method was based on a previously established protocol for glycosaminoglycan (GAG) extraction and detection by Volpi et al. (Nature Protocols, 9, 541-558 (2014)). Briefly, the method consisted of an enzymatic digestion assay using Chondroitinase ABC and Heparinase I-II-III to depolymerize GAGs in the sample into disaccharides. Note that as compared to Volpi et al., where use of a non-specific protease is recommended for biologicial fluid analysis, the method used in the present study omitted the addition of a protease and thus the analysis was limited to the protein-free fraction of GAGs. Note that as compared to Volpi et al., the method used in the present study omitted the step of purifying the GAGs using an anion-exchange resin.

We verified that no-use versus use of an anion-exchange resin increased the extraction yield of GAGs from sample. We performed an experiment in which 9 RCC and 9 healthy urine samples were prepared with the step of purifying the GAGs using an anion-exchange resin (Nᵣₑₛᵢₙ=18) and 6 RCC and 6 healthy urine samples were prepared omitting that step (N_{no resin}=12). In a multivariable linear regression, we estimated that the omission of anion-exchange resin was associated with an increase of the total CS concentration by 2.4-fold (p=0.0004), of the concentration of 0s CS by 1.9-fold (p=0.04), of the concentration of 4s CS by 2.6-fold (p=0.00003), and of the concentration of 6s CS by 3.0-fold (p=0.000003) irrespective of RCC versus healthy status. This suggested that the purification of GAGs using an anion-exchange resin was less effective in terms of GAG extraction yield than direct enzymatic digestion of the samples omitting the use of an anion-exchange resin, particularly for the extraction of sulfated GAG disaccharides.

GAG disaccharides were subsequently labeled using 2-aminoacridone.

The processed samples were then injected into an ultra-high-performance liquid chromatography (UHPLC) coupled with electrospray ionization triple-quadrupole mass spectrometry system (ESI-MS/MS, Waters® 6 Acquity I-class Plus Xevo TQ-S micro) for disaccharide separation and detection. The peaks of 17 GAG disaccharides (listed below) were acquired at pre-specified retention times across six transitions using multiple reaction monitoring (MRM) analysis implemented in the mass spectrometry software (Waters® 9 TargetLynx). We used the mass spectrometry software (Waters® TargetLynx) for peak integration, construction of calibration curves, and quantification. We exported the results processed data in Excel format and imported it into R (4.0.2) for secondary analysis.

The measured GAG profiles (GAGomes) consisted of absolute concentrations for 17 GAG disaccharides, corresponding to 8 different sulfation patterns of chondroitin sulfate (CS) and heparan sulfate (HS), and hyaluronic acid (HA) disaccharide. Specifically, we quantified 8 CS disaccharides (0s CS, 2s CS, 6s CS, 4s CS, 2s6s CS, 2s4s CS, 4s6s CS, Tris CS) and 8 HS disaccharides (0s HS, 2s HS, 6s HS, Ns HS, Ns6s HS, Ns2s HS, 2s6s HS, Tris HS). The GAGome was expanded to include an additional 22 dependent features: the total CS and total HS concentration as the sum of the corresponding disaccharide concentrations, the CS and HS charge, two ratios (4s CS/0s CS and 6s CS/0s CS), and the relative concentration (or mass fraction, in %) of each of the 16 CS and HS disaccharide by normalizing its absolute concentration by the total CS and HS concentration, respectively. For a plasma or urine sample taken at a given visit, the GAGome, therefore, consisted of 39 GAG features.

GAGomes in any-Stage Cancer Versus Physiological Levels.

We next examined if there were differences in GAGome features across each cancer type versus its physiological levels as measured in the healthy subject group. To this end, we used a Bayesian mixed effect linear regression with skewed response model to correlate each GAGome feature with each group as a predictor. We deemed a GAGome feature credibly different from physiological levels in a given cancer type by defining a region of practical equivalence (ROPE) centered on the estimated level across healthy subjects. This analysis highlighted several GAGome features that were altered across multiple cancers. Most notably, we observed an almost universal increase in the urine and plasma concentration of non-sulfated CS (0s CS). We additionally uncovered several cancer type-specific GAGome features, such as a lower plasma CS charge in colorectal carcinoma (CRC) and non-follicular lymphoma (NHL) and elevated urine 2s6s CS in prostate cancer (PCa).

In plasma an increase in non-sulfated (0s) CS was observed in 12 (85%) of 14 cancer types. In plasma, an increase in total CS was observed in 11 (80%) of 14 cancer types. In plasma, 4-sulfated (4s) CS was elevated in genitourinary and respiratory tract cancers (Gen: RCC, BCa, PCa; Resp: HN, NSCLC). In urine, an increase in non-sulfated (0s) CS and HS was observed in 3 (60%) of 5 cancer types. In urine, an increase in total CS was observed in 4 (80%) of 5 cancer types. In urine, an increase in 6-sulfated CS—either monosulfated or disulfated (2s6s) was found in non-small cell lung cancer samples.

Table A below shows certain data demonstrating that GAGome features analysed in the present study are significantly (ROPE <5%) altered in cancer versus healthy samples (plasma or urine samples). A figure of <0.05 in the “ROPE_Percentage” column means a ROPE value of <5%. A positive number in the “difference column” means that the stated GAGome feature was increased in cancer samples versus healthy samples. A negative number in the “difference column” means that the stated GAGome feature was decreased in cancer samples versus healthy samples. “μug·mL”=Total GAG class concentration in microgram/mL. The “_conc” suffix=GAGome feature in absolute concentration (microgram/mL). No suffix=GAGome feature in relative concentration (microgram/microgram total for the relevant GAG group, e.g. CS) (also viewed as “mass fraction”—relative concentration is also explained elsewhere herein). Cancer types are BCa—Bladder cancer; BC-Breast invasive ductal carcinoma; CST—Cervix squamous cell carcinoma; LL—Chronic lymphoid leukaemia; CRC—Colorectal cancer; EC—Endometrial cancer; DG—Diffuse glioma; GNET—Gastro-intestinal endocrine tumors; HN—Head and neck squamous cell carcinoma; NHL—Diffuse Large B-Cell Lymphoma—NSCLC Non-small cell lung cancer; OV—Ovarian cancer; PCa—Prostate cancer; RCC—Renal cell cancer. “95% CI”=95% credibility interval for the value difference in cancer type vs. healthy.

TABLE A

| Cancer | | | | 95% CI | 95% CI | |
|---|---|---|---|---|---|---|
| type | Fluid | GAGome feature | Difference | (Lower) | (Upper) | ROPE_Percentage |
| BC | Plasma | 0 s CS plasma conc | 0.52 | 0.15 | 0.91 | 0.02 |
| BCa | Plasma | ug · ml CS plasma | 0.72 | 0.41 | 1.00 | 0.00 |
| BCa | Plasma | 4 s CS plasma | 0.52 | 0.30 | 0.73 | 0.00 |
| BCa | Plasma | 4 s CS plasma conc | 0.93 | 0.55 | 1.31 | 0.00 |
| CRC | Plasma | 0 s CS plasma conc | 0.68 | 0.24 | 1.13 | 0.00 |
| CRC | Plasma | Charge CS plasma | −0.49 | −0.86 | −0.14 | 0.03 |
| CRC | Plasma | 0 s CS plasma | 0.49 | 0.14 | 0.86 | 0.03 |
| CST | Plasma | 0 s CS plasma conc | 0.72 | 0.41 | 1.03 | 0.00 |
| CST | Plasma | ug · ml CS plasma | 0.56 | 0.10 | 0.99 | 0.03 |
| DG | Plasma | ug · ml CS plasma | 0.74 | 0.23 | 1.23 | 0.00 |
| DG | Plasma | 0 s CS plasma conc | 0.87 | 0.50 | 1.25 | 0.00 |
| EC | Plasma | ug · ml CS plasma | 0.73 | 0.24 | 1.20 | 0.00 |
| EC | Plasma | 0 s CS plasma conc | 0.87 | 0.53 | 1.21 | 0.00 |
| GNET | Plasma | 0 s CS plasma conc | 0.69 | 0.23 | 1.16 | 0.00 |
| GNET | Plasma | ug · ml CS plasma | 0.65 | 0.06 | 1.25 | 0.04 |
| HN | Plasma | ug · ml CS plasma | 0.90 | 1.32 | 0.48 | 0.00 |
| HN | Plasma | 4 s CS plasma conc | 0.94 | 1.39 | 0.45 | 0.00 |
| HN | Plasma | 0 s CS plasma conc | 0.46 | 0.79 | 0.12 | 0.04 |
| LL | Plasma | ug · ml CS plasma | 0.72 | 1.24 | 0.20 | 0.00 |
| LL | Plasma | 0 s CS plasma conc | 0.90 | 1.27 | 0.50 | 0.00 |
| NHL | Plasma | 0 s CS plasma conc | 0.83 | 1.18 | 0.46 | 0.00 |
| NHL | Plasma | Charge CS plasma | −0.45 | −0.13 | −0.78 | 0.04 |
| NSCLC | Plasma | ug · ml CS plasma | 1.09 | 0.81 | 1.37 | 0.00 |
| NSCLC | Plasma | 0 s CS plasma conc | 0.86 | 0.65 | 1.08 | 0.00 |
| NSCLC | Plasma | 4 s CS plasma conc | 0.88 | 0.5 | 1.23 | 0.00 |
| OV | Plasma | ug · ml CS plasma | 0.72 | 0.23 | 1.18 | 0.00 |
| OV | Plasma | 0 s CS plasma conc | 0.82 | 0.5 | 1.15 | 0.00 |
| PCa | Plasma | ug · ml CS plasma | 0.56 | 0.3 | 0.82 | 0.00 |
| PCa | Plasma | 4 s CS plasma | 0.42 | 0.23 | 0.61 | 0.00 |
| PCa | Plasma | 4 s CS plasma conc | 0.67 | 0.31 | 1.02 | 0.00 |
| RCC | Plasma | ug · ml CS plasma | 1.42 | 0.8 | 2.03 | 0.00 |
| RCC | Plasma | 0 s CS plasma conc | 0.99 | 0.56 | 1.4 | 0.00 |
| RCC | Plasma | 4 s CS plasma | 0.93 | 0.44 | 1.41 | 0.00 |
| RCC | Plasma | 4 s CS plasma conc | 1.49 | 0.64 | 2.31 | 0.00 |
| BCa | Urine | ug · ml CS urine | 0.89 | 0.40 | 1.39 | 0.00 |
| BCa | Urine | 0 s CS urine conc | 1.22 | 0.84 | 1.64 | 0.00 |
| BCa | Urine | 4 s CS urine conc | 0.74 | 0.24 | 1.25 | 0.00 |
| BCa | Urine | 6 s CS urine | −0.91 | −1.52 | −0.26 | 0.00 |
| BCa | Urine | 0 s CS urine | 0.53 | 0.12 | 0.96 | 0.03 |
| HN | Urine | ug · ml HS urine | 0.80 | 0.25 | 1.45 | 0.00 |
| HN | Urine | 4 s CS urine conc | 0.68 | 0.19 | 1.18 | 0.00 |
| HN | Urine | Ns HS urine conc | 0.79 | 0.15 | 1.52 | 0.01 |
| HN | Urine | ug · ml CS urine | 0.60 | 0.13 | 1.09 | 0.02 |
| HN | Urine | 0 s HS urine conc | 0.58 | 0.1 | 1.15 | 0.03 |
| NSCLC | Urine | Ns HS urine conc | 1.31 | 0.75 | 1.92 | 0.00 |
| NSCLC | Urine | ug · ml CS urine | 1.21 | 0.75 | 1.69 | 0.00 |
| NSCLC | Urine | ug · ml HS urine | 1.18 | 0.72 | 1.7 | 0.00 |
| NSCLC | Urine | 0 s CS urine conc | 1.23 | 0.87 | 1.61 | 0.00 |
| NSCLC | Urine | 0 s HS urine conc | 1.11 | 0.68 | 1.59 | 0.00 |
| NSCLC | Urine | 2 s6 s CS urine conc | 1.05 | 0.51 | 1.6 | 0.00 |
| NSCLC | Urine | 4 s CS urine conc | 1.11 | 0.66 | 1.63 | 0.00 |
| NSCLC | Urine | 6 s CS urine | −0.78 | −1.28 | −0.31 | 0.00 |
| NSCLC | Urine | 6 s CS urine conc | 0.71 | 0.2 | 1.25 | 0.00 |
| PCa | Urine | 2 s6 s CS urine | 0.61 | 0.06 | 1.15 | 0.04 |
| RCC | Urine | Charge CS urine | −1.04 | −1.56 | −0.55 | 0.00 |
| RCC | Urine | ug · ml HS urine | 0.94 | 0.53 | 1.43 | 0.00 |
| RCC | Urine | 0 s CS urine | 1.09 | 0.62 | 1.59 | 0.00 |
| RCC | Urine | 0 s CS urine conc | 1.08 | 0.75 | 1.45 | 0.00 |
| RCC | Urine | 0 s HS urine conc | 0.60 | 0.22 | 1 | 0.00 |
| RCC | Urine | 6 s CS urine | −1.28 | −1.78 | −0.8 | 0.00 |
| RCC | Urine | ug · ml CS urine | 0.51 | 0.14 | 0.9 | 0.02 |

Further details of the statistical analysis and Bayesian estimation (ROPE estimation) are provided here: We carried out estimation of group differences in GAG feature by Bayesian estimation and equivalence testing, BEST (Kruschke, J. Exp Psychol Gen, 142, 573-603 (2013)). In short, we modelled each individual standardized GAGome feature as a response with a skew-normal distribution in a mixed effects model, where diagnosis was a fixed effect and experimental batch was treated as a random factor. We modeled the group-specific variances as a multiplicative interaction between the cancer-type and experimental batch. We used non-informative priors (Kruschke, J. Exp Psychol Gen, 142, 573-603 (2013)) for all predictors. We considered the convergence of Bayesian estimation acceptable if the effective sample size (ESS)>5000 and the potential scale reduction factor R<1.001. We used the posterior samples to compute the 95% credible interval (95% CI) of group medians for each GAGome feature conditional on the diagnosis. Next, we computed the 95% credible interval (95% CI) for the difference in medians of each cancer diagnosis versus healthy. We deemed that a GAGome feature was correlated with the diagnosis vs health if 95% CI of the difference in means did not cross 0 and no more than 5% fell inside the pre-specified region of practical equivalence (ROPE) interval around 0. We defined the ROPE boundaries as 0.2 of the overall standardized mean. Bayesian estimation was carried out using the brms (2.14.4) package (Bürkner, Journal of Statistical Software, 80, 1-28 (2017); Bürkner, The R journal, 10, 395-411 (2018)), and tidybayes (Matthew Kay, Zenodo, 2020; https://zenodo.org/record/4284191) packages (version) in R (4.0.3).

### Development and Validation of GAG Scores as Pan-Cancer Biomarker

Having confirmed that there were several shared and at least one altered GAGome feature in all cancer types, we sought to identify a minimal set of GAGome features to robustly discriminate between any-type cancer from healthy controls.

We first split the data set into a discovery (60%) and validation (40%) sets so to ensure that there were at least 5 samples for each cancer type in the validation set (Table 2).

TABLE 2
Number of samples with urine-only GAGome features, plasma-
only GAGome features or both urine and plasma (“combined”) GAGome
features assigned to the discovery versus validation sets
by group. We randomly designated each sample to either discovery
or validation set, with a 60%:40% ratio and the constraint
that there at least 5 samples for each cancer type were
designated to the validation set.

| | Discovery | | | Validation | | |
|---|---|---|---|---|---|---|
| Group | Urine | Blood | Combined | Urine | Blood | Combined |
| H | 205 | 255 | 204 | 135 | 170 | 135 |
| BC | 0 | 16 | 0 | 0 | 12 | 0 |
| BCa | 24 | 24 | 24 | 22 | 23 | 22 |
| CRC | 0 | 15 | 0 | 0 | 12 | 0 |
| CST | 0 | 14 | 0 | 0 | 14 | 0 |
| DG | 0 | 28 | 0 | 0 | 12 | 0 |
| EC | 0 | 23 | 0 | 0 | 7 | 0 |
| GNET | 0 | 9 | 0 | 0 | 5 | 0 |
| HN | 9 | 9 | 9 | 8 | 8 | 8 |
| LL | 0 | 13 | 0 | 0 | 5 | 0 |
| NHL | 0 | 16 | 0 | 0 | 14 | 0 |
| NSCLC | 26 | 45 | 26 | 24 | 38 | 24 |
| OV | 0 | 20 | 0 | 0 | 10 | 0 |
| PCa | 39 | 49 | 24 | 17 | 31 | 8 |
| RCC | 34 | 31 | 27 | 17 | 14 | 12 |

H—healthy;
BC—Breast cancer;
BCa—Bladder cancer;
CRC—Colorectal cancer;
CST—Cervical cancer;
DG—Diffuse Glioma;
EC—Endometrial Carcinoma;
GNET—Small Intestinal Neuroendocrine Tumor;
HN—Head and Neck Cancer;
LL—B-cell Leukemia;
NHL—Non-follicular Lymphoma;
NSCLC—Non-small-cell Lung Carcinoma;
OC—Ovarian Carcinoma;
PCa—Prostate cancer;
RCC—Renal Cell Carcinoma

Using the discovery set, we used projection predictive variable selection (Piironen, J., Paasiniemi, M. and Vehtari, A. ‘Projective inference in high-dimensional problems: Prediction and feature selection’, Electronic Journal of Statistics, 14, 2155-2197 (2020)) to select relevant features independently in urine, plasma, and combined GAGomes. Next, we fit three reference (plasma-only, urine-only, and combined) Bayesian multivariable logistic regressions with cancer (aggregating all cancer types) vs healthy as a response, and standardized detectable GAGome features as predictors. Note that the plasma-only and urine-only models used as inputs the GAGome features measured in plasma samples only or urine samples only, respectively, whereas the combined model used as inputs both plasma and urine GAGome features. We used heavy-tailed standard t-distribution (df=3) as a prior on the intercept and coefficients for all predictors. We fit the model using rstanrm package (2.21.1) with 4 chains for a total of 4000 iterations (2000 warm-up). The R² of urine, plasma and combined reference models was of 0.31, 0.45 and 0.66, respectively. Next, we carried out the variable selection using leave-one-out cross validation forward selection using the cv_varsel function from the projpred package version 2.0.2 (projpred: Projection Predictive Feature Selection (https://mc-stan.org/projpred/). We selected the sub-model of a minimal size such that the estimated difference of sum of log predictive densities (ELPD) between the reference model and sub-model was at most one standard error away from the zero (default). Where necessary, we further constrained the size of the model such that the number of predictors never exceeded the 1/10 of the cancer sample number in the discovery set. The constraints on the maximal number of parameters were 31, 13, 11 for plasma, urine, and combined, respectively. We then selected and projected the final set of sub-models, with either the default suggested model size (plasma, 3 features), or based on the maximal prespecified number of features, which was 13 for urine, and 11 for the combined model. Finally, we projected the 400 draws of the sub-model of the selected size, and predicted the response using draws of the linear predictor (proj_linpred function, averaged over all parameters). The response, which corresponds to the log-odds for any-cancer vs. healthy controls, is defined as the pan-cancer GAG score. If only the three plasma GAGome features were used as inputs, the GAG score is the plasma pan-cancer GAG score. Conversely, if the 13 urine GAGome features were used as inputs, the GAG score is the urine pan-cancer GAG score. Finally, if the 11 combined GAGome features were used as inputs, the GAG score is the combined pan-cancer GAG score.

The three GAGome features used for the plasma score were 0s CS (μg/ml), 4s/0s CS and 4s CS (%), all measured in a plasma sample.

The 13 GAGome features used for the urine score were 0s HS (μg/ml), 6s CS (%), Ns HS (μg/ml), 2s6s CS (%), Ns HS (%), 0s HS (%), Total HS (μg/ml), 4s CS (%), 0s CS (%), 6s/0s CS, 2s6s CS (μg/ml), 4s CS (μg/ml) and Total CS (μg/ml), all measured in a urine sample.

The 11 GAGome features used for the combined score were plasma total CS (μg/ml), urine 0s HS (μg/ml), urine Ns HS (μg/ml), urine 0s CS (%), plasma 4s CS (%), urine 2s6s CS (%), urine Ns HS (%), urine 0s HS (%), urine total HS (μg/ml), urine charge CS and plasma 0s CS (μg/ml).

In a given GAG score, a certain GAGome feature is positively (negatively) associated with any-type cancer vs. healthy status if the corresponding coefficient is positive (negative) conditional to the value of all other GAGome feature in that GAG score.

For each pan-cancer GAG score, we estimated metrics of discrimination (in terms of area under the receiver operating characteristic curve (ROC), AUC) and clinical usefulness (sensitivity at 98% specificity). Confidence intervals for sensitivity at 98% specificity were calculated by binomial approximation.

The pan-cancer GAG scores distinguished any-type cancer from healthy subjects with an AUC=0.83 (95% CI=0.80-0.86) in discovery and 0.84 (95% CI=0.80-0.88) in validation for plasma (FIG. 1A and FIG. 1B); AUC=0.88 (95% CI=0.85-0.93) in discovery and 0.82 (95% CI=0.76-0.87) in validation for urine (FIG. 1C and FIG. 1D); and AUC=0.92 (95% CI=0.90-0.96) in discovery and 0.86 (95% CI=0.81-0.92) in validation for combined plasma and urine (FIG. 1E and FIG. 1F). In the validation set, the sensitivity to any-stage cancer for the plasma, urine, and combined pan-cancer GAG score was 38.6% (95% CI=18.0%-47.8%), 34.6% (95% CI=22.7%-51.1%), and 40.5% (95% CI=25.7%-54.1%), respectively (FIG. 1G). In the subset of stage I/low-grade tumors, the sensitivities were 30.9% (95% CI=8.8%-47.1%), 33.3% (95% 12.5%-54.2%), and 33.3% (95% CI=9.5%-61.9%), for plasma, urine and combined respectively. All three scores showed similar sensitivities to stage I-II and stage I-III in the validation subsets revealing a weak dependency between GAGome alterations and tumor stage or grade (FIG. 1G). The sensitivity was comparable across individual cancer types, ranging from 21% in breast cancer and 71% in renal cell cancer. In addition, since all ROC curves were approximately symmetric, an opposite scenario in which the sensitivity is fixed at 98% yielded specificities in the 20% to 40% range.

### Glycosaminoglycans can Predict Tissue-of-Origin

Given that presence of distinctive GAGome patterns across cancer types, we investigated if combined plasma and urine GAGomes could be used to identify cancer tissue of origin (TOO). We carried out tissue of origin analysis (TOO) on the cancer subset of the cohort, where TOO was defined as the cancer type. We preselected certain GAG forms (median measured concentration was higher than 0.1 μg/mL). We trained a multinomial Bayesian Additive Regression Trees (BART) (R. Sparapani, et al. Journal of Statistical Software. 97, 1-66 (2021)) on the cancer samples in the discovery set (60%, N=110 across 5 cancer types), where the cancer type was the response and pre-selected GAGs were predictors. We assigned the category with the highest mean posterior probability as the predicted category (FIG. 2). We validated the prediction accuracy on the validation set (remaining 40%, N=74). Confidence intervals for accuracy and balanced accuracy were calculated by 5000 bootstrapped replicates using normal approximation. In the validation set, the balanced accuracy of classification was 74.3% (95% CI=68.1%-80.3%) (FIG. 1H). When grouping tumors into respiratory tract (NSCLC and HN) versus genitourinary (RCC, PCa and BCa), the accuracy to predict TOO was 89.2% (95% CI: 82.2%-96.4%).

Pan-Cancer GAG Scores were Independent Predictors of Overall Survival

To assess whether altered GAGome features in cancer were suggestive of aggressive tumor biology, we correlated each pan-cancer GAG score with overall survival (OS). From the date of sample collection, the median follow-up time was 17 months in the plasma cohort (N=370 across 13 cancer types, range: 14-47 per type; N_{deaths}=82, range: 1-18 per type) and 15 months in the urine cohort (N=162 across 4 cancer types, range: 17-50 per type; N_{deaths}=33, range: 4-13 per type). In multivariable Cox regression, both the plasma and the urine pan-cancer GAG score were independent predictors of OS (HR=1.30, 95% CI: 1.08-1.57, p=0.006 and HR=1.53, 95% CI: 1.07-2.18, p=0.019, respectively), after adjusting for cancer type, age, gender, and late-stage/high-grade versus early-stage/low-grade disease.

Next, we used maximally selected rank statistics (p<0.01) to determine an optimized cut-off for the plasma, urine, and combined pan-cancer GAG scores and so dichotomize patients in “High” vs. “Low” risk groups depending on if their individual score was above or below the cut-off. For all three scores, the risk groups correlated with OS across and within cancer types (HR=1.87 (95% CI=1.36-2.57), p<0.00013 in plasma, FIGS. 3A and 4A; HR=2.50 (95% CI=1.50-4.16), p<0.005 in urine, FIGS. 3B and 4B; HR=2.66 (95% CI=1.60-4.44), p<0.000168 when combined, FIGS. 3C and 4C). These findings implicate GAGome alterations with aggressive cancer phenotypes.

### GAGomes Dynamics in an In Vivo Model of Cancer Progression

We sought to validate whether the alterations in the GAGomes so far attributed to any-type cancer were indeed mechanistically linked to the onset and progression of cancer in vivo. We carried out longitudinal measurements of urine and plasma GAGomes in BALB/c (BALB/cAnNCrl) mice in which Renca renal adenocarcinoma were induced orthotopically on day zero (D0) (N=20 in 10 metabolic cages, FIG. 5A). The kidney carrying the tumor was resected on day 7 and the mice were sacrificed on day 20. This model was chosen since it recapitulates cancer progression from localized to metastatic recurrence after surgery (P. Sobczuk, et al., Transl Oncol. 13, 100745 (2020)). In a principal component analysis (PCA), we observed alterations in the plasma GAGomes and to a lesser extent in the urine GAGomes consistent with progression from baseline (day 0) to localized growth (day 6) to post-operative resection (day 8) to metastasis (day 20) (FIG. 5B). Consistent with the above-observed patterns in human cancer samples, we reported a credible linear increase in non-sulfated CS (0s CS) across the timepoints in both plasma and urine (% change at metastasis vs. baseline=148% [95% credible interval: +91%, +231%] in plasma and 116% [95% credible interval: +39%, +211%] in urine, FIG. 5C). This suggests that the GAGome alterations captured in the pan-cancer GAG scores were causally linked to cancer initiation and progression.

### Further Details Regarding the Mouse Study are as Follows:

In short, 28 female BALB/c (BALB/cAnNCrl) mice, 5-6 weeks old at reception, were used in the experiment—3 as controls. RenCa tumors were induced on day zero (D0) orthotopically on 25 female BALB/c mice under anesthesia. Briefly, the animal abdomen was opened through a median incision under aseptic conditions. 5×105 murine renal adenocarcinoma (RenCa) tumor cells (American Type Culture Collection, USA), in 25 μL of Roswell Park Memorial Institute (RPMI) medium, were slowly injected in subcapsular space of the left kidney. At day seven (D7), the abdomen of mice was opened and the kidney containing injected RenCa cells was resected.

Blood was collected from 20 mice at each time point. Drop-outs due to compassionate termination were replaced. Blood (50 μL per sample) was collected into K2 EDTA tube by jugular venipuncture. Intra-cardiac blood collection was conducted as a terminal procedure under deep isoflurane gas anesthesia. Blood was collected from animals at the following time points on D-1 (24h before engraftment), D6 (24h before kidney resection), D8 (24h after kidney resection) and D20 (day of mice termination).

Therefore, each mouse generated up to 4 plasma samples. Blood was collected into collection tubes with anticoagulant (K2 EDTA). Tubes were centrifuged (2000 g, 10 minutes, room temperature) to obtain plasma. Plasma samples were stored in propylene tubes at −20° C. until shipment.

Urine was collected from 20 mice split in 10 metabolic cages. Drop-outs due to compassionate termination were replaced. The animals were kept in metabolic cages for the collection of pooled urine of two mice per cage for 24 hours at +4° C. All urine was collected from animals at the following time points from D-2 to D-1 (24h before OT engraftment), D5 to D6 (24h before kidney resection), D7-D8 (24h after kidney resection) and D19 to D20 (24h before mice termination) was collected in propylene tubes and stored at −20° C. until shipment. Each group of 2 mice generated 4 urine samples. All surviving mice were terminated on D20 as described above.

### DISCUSSION

Compared to physiological levels, we observed widespread changes in the GAGomes of cancer patients. Such changes were observed already at stage I. Elevation of non-sulfated CS was particularly noted. These trends were recapitulated in an in vivo mouse model, causally linking them to cancer shortly after initiation. Few small studies have investigated biofluidic GAGomes in disease. Among these, respiratory failure and septic shock did not alter plasma or urine non-sulfated CS remarkably despite the fact that these conditions directly affect the GAG-rich endothelial glycocalyx of lungs and kidneys, respectively. Taken together, this suggests a tumor-related origin for the increase of non-sulfated CS.

Here, we harnessed the GAGome alterations attributed to cancer to design a liquid biopsy test to detect multiple types. Unlike genomics- and proteomics-based detection assays that survey an entire landscape of potential alterations, patient GAGomes make up a comparatively miniscule feature set. GAGome features were capable to extract meaningful information about the spatial and temporal status of cancer from an early stage. From a practical perspective, this results in a relatively low assay complexity and hence cost, a more feasible implementation in high-volume settings such as screening, and a robust predictive performance. Using the pan-cancer GAG scores, the sensitivity to stage I/low-grades tumors ranged between 31%-33% at 98% specificity, comparable or superior to recently reported genomics-based assays. Notably, while these genomics-based assays perform poorly in cancers that shed little cell-free DNA e.g. genitourinary and brain tumors, we observed that GAGomes were altered in all cancer types tested, including low and high-grade gliomas and in renal cell carcinoma.

The metabolic nature of GAGomes and their ability to detect several cancer types at an early stage makes them very useful and makes them suitable for a first-pass stand-alone test or as a combination test in a multi-cancer screening setting.

### EXAMPLE 2

Study design, patient recruitment, study population characteristics, sample collection and sample processing, and GAGome measurements (GAG measurements) were essentially the same as presented in Example 1. Main differences are outlined below.

### Samples

Across all cohorts, we successfully analyzed a total of 969 plasma samples and 560 urine, so divided: for the case arm, 517 plasma samples in 14 cancer types and 220 urine samples in 5 cancer types; and for the control arm 452 plasma and 340 urine samples. A subset of 184 cases (5 cancer types) and 339 healthy controls had combined plasma and urine samples available.

### Development of Pan-Cancer GAG Scores

We aimed to identify a minimal subset of GAGome features (also referred to herein as GAG features or GAG properties) which were informative for discrimination between cancer vs healthy subjects. To this end, we used projection predictive variable selection to select relevant features independently in urine, plasma, and combined GAGomes. First, we fit three reference (plasma-only, urine-only, and combined) Bayesian multivariable logistic regressions with cancer (aggregating all cancer types) vs healthy as a response, and standardized detectable GAGome features as predictors. We used heavy-tailed standard t-distribution (df=3) as a prior on the intercept and coefficients for all predictors. We fit the model using rstanrm package (2.21.1) with 4 chains for a total of 4000 iterations (2000 warmup).

Next, we carried out the variable selection using leave-one-out cross validation forward selection using the cv_varsel function from the projpred package version 2.0.223,24,39 (Kruschke, J. Exp Psychol Gen, 142, 573-603 (2013)). We selected the sub-model of a minimal size such that the estimated difference of sum of log predictive densities (ELPD) between the reference model and sub-model was at most one standard error away from the zero (default). Where necessary, we further constrained the size of the model such that the number of predictors never exceeded the 1/10 of the cancer sample number in the training cohort. We then selected and projected the final set of sub-models, with the default suggested model size (plasma—3 features, urine—13 features, combined—14 features).

Finally, for each model, we projected the 400 draws of the sub-model of the selected size, and predicted the response using draws of the linear predictor (proj_linpred function, averaged over all parameters). The effect size of the response, called pan-cancer GAG score, was predicted as log-odds of any-type cancer. Confidence intervals for sensitivity at 95% specificity were calculated by binomial approximation.

If the three plasma GAGome features were used as inputs, the GAG score is the plasma pan-cancer GAG score. Conversely, if the 13 urine GAGome features were used as inputs, the GAG score is the urine pan-cancer GAG score. Finally, if the 14 combined GAGome features were used as inputs, the GAG score is the combined pan-cancer GAG score.

The three GAGome features used for the plasma score were 0s CS [μg/mL], 4s CS [%] and 4s/0s CS, all measured in a plasma sample.

The 13 GAGome features used for the urine score were 0s HS [μg/mL], 6s CS [%], Ns HS [%], 0s HS [%], 2s6s CS [%], Total HS [μg/mL], 4s CS [%], 0s CS [%], 6s/0s CS, 4s CS [μg/mL], 6s CS [μg/mL], Total CS [μg/mL] and 0s CS [μg/mL], all measured in a urine sample.

The 14 GAGome features used for the combined score were Total CS plasma [μg/mL], 0s HS urine [μg/mL], Ns HS urine [%], 0s CS urine [%], 4s/0s CS plasma, Charge CS urine [−], 0s HS urine [%], 4s CS urine [μg/mL], Total CS urine [μg/mL], 6s/0s CS urine, 0s CS urine [μg/mL], 4s CS urine [%], 6s CS urine [%] and 6s CS urine [μg/mL].

Results with Pan-Cancer GAG Scores

For each pan-cancer GAG score, we estimated metrics of discrimination (in terms of area under the receiver operating characteristic curve (ROC), AUC) and clinical usefulness (sensitivity at 95% specificity). The pan-cancer GAG scores distinguished any-type cancer from healthy subjects with an AUC=0.83 (95% CI=0.8-0.86) for plasma (FIGS. 6A and D), AUC=0.88 (95% CI=0.85-0.91) for urine (FIGS. 6B and D), and AUC=0.93 (95% CI=0.9-0.95) for combined plasma and urine (FIGS. 6C and D). The sensitivity to any-stage cancer for the plasma, urine, and combined pan-cancer GAG score was 46.2% (95% CI=41.9%-50.6%), 66.8% (95% CI=60.2%-73.0%), and 65.8% (95% CI=58.4%-72.6%) at 95% specificity, respectively. In the subset of stage I/low-grade disease, the sensitivity at 95% specificity was 41.6% (95% CI=34.2%-49.2%), 62.3% (95% CI=47.9%-75.2%), and 61.4% (95% CI=45.5%-75.6%), for plasma, urine and combined scores respectively. At 99% specificity, the sensitivity was 25.7%, 25.0% and 35.3% for plasma, urine, and combined scores, respectively. All three scores showed a weak dependency between free GAGome alterations and tumor stage or grade with a slight sensitivity increase in stages I-II and further in stages I-III. Overall, we observed a similar sensitivity of each score across individual cancer types. The top detected types were NHL; CRC, and chronic lymphocytic leukemia (LL) for the plasma pan-cancer GAG score (range: 23.4% in bladder cancer to 66.7% in NHL, LL, and CRC), and RCC and non-small cell lung cancer (NSCLC) for the urine or combined scores (range: 47.1% in head and neck squamous cell carcinoma to 82.4%-84.6% in RCC, for urine and combined respectively). The following individual types of cancer, NHL, LL, DG, CRC, EC, NSCLC, OV, CST, BC, GNET, RCC, HN, PCa and BCa (abbreviations are as per elsewhere herein, e.g. as per in Example 1), were also analysed individually using the plasma score and it was found that, for each of the individual cancer types, the median plasma score was clearly altered (increased) in comparison to healthy samples (data not shown). The following individual types of cancers, RCC, PCa, NSCLC, HN, BCa, were also analysed individually using the urine score and it was found that, for each of the individual cancer types, the median urine score was clearly altered (increased) in comparison to healthy samples (data not shown). The following individual types of cancers, RCC, PCa, NSCLC, HN, BCa, were also analysed individually using the combined score and it was found that, for each of the individual cancer types, the median combined score was clearly altered (increased) in comparison to healthy samples (data not shown). Taken together, these findings indicate that free GAGomes were significantly altered from physiological levels across early- and late-stage cancers and would be useful for multi-cancer early detection.

### Internal Validation of the Pan-Cancer GAG Scores

We validated the variable selection procedure by bootstrap analysis. To this end, we analyzed 500 bootstraps for plasma, and 1000 for urine and combined datasets. In each bootstrap, we first fit the reference logistic Bayesian regression model. We used the same priors and fitting parameters as described above (t distribution priors with df=3, 4000 iterations with 2000 warmup samples). Second, we carried out the projection predictive variable selection using leave-one-out cross validation. Projection used 400 samples, with projected model size determined automatically by ELPD or set to 1/10 of the number of cases in the original dataset. Finally, we predicted the response (log-odds of any-type cancer) using draws of the linear predictor for the three datasets: the bootstrap, assessment dataset (samples left out of the bootstrap) and the original dataset. We recorded the selected model size, AUC, sensitivity at 95% specificity, and scaled Brier metric for the full and projected model on the bootstrapped, original and assessment dataset.

### Correlation Between Pan-Cancer GAG Scores and Overall Survival

To assess whether altered GAGome features in association with cancer were suggestive of aggressive tumor biology, we correlated each pan-cancer GAG score with overall survival (OS). From the date of sample collection, the median follow-up time was 17 months in the plasma cohort (N=370 across 13 cancer types, range: 14-47 per type; Ndeaths=82, range: 1-18 per type), 15 months in the urine cohort (N=162 across 4 cancer types, range: 17-50 per type; Ndeaths=33, range: 4-13 per type), and 15 months in the combined cohort (N=152 across 4 cancer types, range: 17-50 per type; Ndeaths=33, range: 4-13 per type). In multivariable Cox regression, the plasma, as well as the urine and the combined pan-cancer GAG scores were independent predictors of OS (hazard ratio [HR]=1.29, 95% CI: 1.06-1.56, p=0.0009 for plasma; HR=1.79, 95% CI: 1.27-2.53, p=0.0009 for urine; HR=1.91, 95% CI: 1.33-1.73, p=0.0004, for combined), after adjusting for cancer type, age, gender, and stage IV/high-grade disease. These findings implicated GAGome alterations with aggressive cancer phenotypes. Further, they suggested that patients with a score below the 95%-specificity cut-off—or in other words, undetected when using the pan cancer GAG scores—might have better prognosis. To verify this for each score, we dichotomized patients in “High” vs. “Low” groups depending on whether their individual score was above or below the score-specific 95%-specificity cut-off. For the plasma and urine GAG scores, Kaplan-Meier survival analyses suggested that “Low”-risk (undetected) patients had 39%-50% lower risk of death than “High”-risk (detected) patients (unadjusted HR=0.61 (95% CI=0.44-0.85), p=0.0031 in plasma FIG. 6E; HR=0.50 (95% CI=0.25-0.98), p=0.0441 in urine FIG. 6F). The results for the survival difference between the groups is shown in FIG. 6G for the combined GAG score (unadjusted HR=0.86 (95% CI=0.50-1.49), p=0.595, FIG. 6G). Critically, we observed a lower risk of death both in the stage I-III/low grade subset as well as in the stage IV/high-grade subset suggesting an independent correlation between free GAGomes and prognosis. An alternative dichotomization with a “High” vs. “Low”-risk cut-off optimized using maximally selected rank statistic resulted in statistically significant correlations for all three scores with OS, across and within cancer types. Cumulatively, these survival analyses suggest that patients who would go undetected using the pan-cancer GAG scores have a better prognosis and feature a less aggressive cancer phenotype independent of tumor stage and grade.

1. A method of screening for cancer in a subject, said method comprising determining the level and/or chemical composition of the protein-free fraction of one or both of the glycosaminoglycans (GAGs) chondroitin sulfate (CS) and heparan sulfate (HS) in a body fluid sample, wherein said sample has been obtained from said subject.

2. The method of claim 1, wherein an altered level and/or chemical composition of chondroitin sulfate (CS) and/or heparan sulfate (HS) in said protein-free fraction in comparison to a control level and/or chemical composition is indicative of cancer in said subject.

3. The method of claim 1, wherein said determination of the chemical composition comprises determining the level in said protein-free fraction of one or more GAG properties selected from the group consisting of: one or more (or all) of the specific sulfated or unsulfated forms of CS or HS disaccharides, charge HS, charge CS, the total concentration of CS or the total concentration of HS.

4. The method of claim 1, wherein said determination of the chemical composition comprises determining the level in said protein-free fraction of one or more (or all) of the GAG properties selected from the group consisting of: the specific sulfated or unsulfated forms of CS or HS disaccharides selected from the group consisting of: Os CS, 2s CS, 6s CS, 4s CS, 2s6s CS, 2s4s CS, 4s6s CS, Tris CS, Os HS, 2s HS, 6s HS, 2s6s HS, Ns HS, Ns2s HS, Ns6s HS, Tris HS, the ratio of 4s CS to 6s CS, the ratio of 6s CS to Os CS and the ratio of 4s CS to Os CS; charge HS; charge CS; the total concentration of CS (CS tot); and the total concentration of HS (HS tot).

5. The method of claim 1, wherein said body fluid sample is blood (e.g. plasma) and/or urine.

6. The method of claim 1, wherein said determination of the chemical composition comprises determining the level in said protein-free fraction of (i) one or more (or all) of the GAG properties selected from the group consisting of: the specific sulfated or unsulfated forms of CS or HS disaccharides selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 6s CS, absolute concentration of 2s6s CS, relative concentration of 4s CS, relative concentration of 2s6s CS, relative concentration of 6s CS, relative concentration of Os HS, relative concentration of Ns HS; the ratio of 6s CS to Os CS; the ratio of 4s CS to Os CS; CS tot; HS tot; charge CS; or
(ii) one or more (or all) of the GAG properties selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 6s CS, absolute concentration of 2s6s CS, relative concentration of 4s CS, relative concentration of 2s6s CS, relative concentration of 6s CS; CS tot; HS tot; charge CS; or
(iii) one or more (or all) of the GAG properties selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 2s6s CS, relative concentration of 4s CS, relative concentration of 2s6s CS, relative concentration of 6s CS, relative concentration of Os HS, relative concentration of Ns HS; the ratio of 6s CS to Os CS; the ratio of 4s CS to Os CS; CS tot; HS tot; charge CS.

7. The method of claim 1, wherein said determination of the chemical composition comprises determining the level in said protein-free fraction of one or more (or all) of the GAG properties selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 2s6s CS, relative concentration of 4s CS, relative concentration of 2s6s CS, relative concentration of 6s CS, relative concentration of Os HS, relative concentration of Ns HS, absolute concentration of 6s CS; the ratio of 6s CS to Os CS; the ratio of 4s CS to Os CS; CS tot; HS tot; charge CS.

8. The method of claim 1, wherein said body fluid sample is blood (e.g. plasma) and said determination of the chemical composition comprises determining the level in said protein-free fraction of
(i) one or more (or all) of the GAG properties selected from the group consisting of: the specific sulfated or unsulfated forms of CS or HS disaccharides selected from the group consisting of: absolute concentration of Os CS, relative concentration of Os CS, absolute concentration of 4s CS, relative concentration of 4s CS; the ratio of 4s CS to Os CS; CS Tot; charge CS; or
(ii) one or more (or all) of the GAG properties selected from the group consisting of: the specific sulfated or unsulfated forms of CS or HS disaccharides selected from the group consisting of: absolute concentration of Os CS, relative concentration of Os CS, absolute concentration of 4s CS, relative concentration of 4s CS; CS Tot; charge CS; or
(iii) one or more (or all) of the GAG properties selected from the group consisting of: the specific sulfated or unsulfated forms of CS or HS disaccharides selected from the group consisting of: absolute concentration of Os CS, relative concentration of 4s CS; the ratio of 4s CS to Os CS.

9. The method claim 1, wherein said body fluid sample is urine and said determination of the chemical composition comprises determining the level in said protein-free fraction of
(i) one or more (or all) of the GAG properties selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 6s CS, absolute concentration of 2s6s CS, relative concentration of 4s CS, relative concentration of 2s6s CS, relative concentration of 6s CS, relative concentration of Os HS, relative concentration of Ns HS; the ratio of 6s CS to Os CS; CS tot; HS tot; charge CS; or
(ii) one or more (or all) of the GAG properties selected from the group consisting of: the specific sulfated or unsulfated forms of CS or HS disaccharides selected from the group consisting of: absolute concentration of 4s CS, absolute concentration of Os CS, relative concentration of Os CS, absolute concentration of Os HS, absolute concentration of Ns HS, absolute concentration of 6s CS, absolute concentration of 2s6s CS, relative concentration of 2s6s CS, relative concentration of 6s CS; CS tot; HS tot; charge CS; or
(iii) one or more (or all) of the GAG properties selected from the group consisting of: the specific sulfated or unsulfated forms of CS or HS disaccharides selected from the group consisting of: absolute concentration of 4s CS, relative concentration of Os CS, absolute concentration of Os HS, absolute concentration of Ns HS, absolute concentration of 2s6s CS, relative concentration of 4s CS, relative concentration of 2s6s CS, relative concentration of 6s CS, relative concentration of Os HS, relative concentration of Ns HS; the ratio of 6s CS to Os CS; CS tot; HS tot.

10. The method of claim 1, wherein said body fluid sample is urine and said determination of the chemical composition comprises determining the level in said protein-free fraction of one or more (or all) of the GAG properties selected from the group consisting of: the specific sulfated or unsulfated forms of CS or HS disaccharides selected from the group consisting of: absolute concentration of Os HS, relative concentration of 6s CS, relative concentration of Ns HS, relative concentration of Os HS, relative concentration of 2s6s CS, relative concentration of 4s CS, relative concentration of Os CS, absolute concentration of 4s CS, absolute concentration of 6s CS, absolute concentration of Os CS; the ratio of 6s CS to Os CS;
CS tot; HS tot.

11. The method of claim 1, wherein the chemical composition of said one or both of said GAGs in said protein-free fraction of a blood (e.g. plasma) sample is determined and the chemical composition of said one or both of said GAGs in said protein-free fraction of a urine sample is performed, preferably
wherein determination of the chemical composition of said protein-free fraction of said urine sample comprises determining the level in said protein-free fraction of one or more (or all) of the GAG properties selected from the group consisting of: relative concentration of Os CS, absolute concentration of Os HS, absolute concentration of Ns HS, relative concentration of 2s6s CS, relative concentration of Os HS, relative concentration of Ns HS; HS tot; and charge CS; and
wherein determination of the chemical composition of said protein-free fraction of said blood (e.g. plasma) sample comprises determining the level in said protein-free fraction of one or more (or all) of the GAG properties selected from the group consisting of: absolute concentration of Os CS; relative concentration of 4s CS; and CS tot.

12. The method of claim 1, wherein the chemical composition of said one or both of said GAGs in said protein-free fraction of a blood (e.g. plasma) sample is determined and the chemical composition of said one or both of said GAGs in said protein-free fraction of a urine sample is performed,
wherein determination of the chemical composition of said protein-free fraction of said urine sample comprises determining the level in said protein-free fraction of one or more (or all) of the GAG properties selected from the group consisting of: absolute concentration of Os HS, relative concentration of Ns HS, relative concentration of Os CS, relative concentration of Os HS, absolute concentration of 4s CS, absolute concentration of Os CS, relative concentration of 4s CS, relative concentration of 6s CS, absolute concentration of 6s CS; the ratio of 6s CS to Os CS; CS tot; and charge CS; and
wherein determination of the chemical composition of said protein-free fraction of said blood (e.g. plasma) sample comprises determining the level in said protein-free fraction of one or both of the GAG properties selected from the group consisting of: the ratio of 4s CS to Os CS; and
CS tot.

13. The method of claim 1, wherein the chemical composition of said one or both of said GAGs in said protein-free fraction of a blood (e.g. plasma) sample is determined and the chemical composition of said one or both of said GAGs in said protein-free fraction of a urine sample is performed,
wherein determination of the chemical composition of said protein-free fraction of said urine sample comprises determining the level in said protein-free fraction of one or more (or all) of the GAG properties selected from the group consisting of: absolute concentration of Os HS, absolute concentration of Ns HS, absolute concentration of 4s CS, absolute concentration of 6s CS; and
wherein determination of the chemical composition of said protein-free fraction of said blood (e.g. plasma) sample comprises determining the level in said protein-free fraction of absolute concentration of Os CS.

14. The method of claim 1, wherein determination of the level and/or chemical composition of said protein-free fraction of said body fluid sample comprises determining the level in said protein-free fraction of one or both of the GAG properties selected from the group consisting of: the absolute concentration of Os CS and CS Tot.

15. The method of claim 1, wherein said method comprises determining the level in said protein-free fraction of the absolute concentration of Os CS and wherein an increase in the absolute concentration of Os CS, in comparison to a control level, is indicative of cancer.

16. The method of claim 3, wherein the chemical composition may be expressed in terms of score, said score being based on the measured level of one or more (preferably more than one) or all of said GAG properties.

17. The method of claim 1, wherein said method comprises determining the level of more than one of said GAG properties, preferably said method comprises determining the level of two or more, three or more, four or more, or all, of said GAG properties.

18. The method of claim 1, wherein said sample has been obtained from said subject and has been subjected to at least one processing step prior to determining said level and/or chemical composition.

19. The method of claim 3, wherein the levels of one or more of the specific sulfated or unsulfated forms of CS or HS disaccharides are determined, and wherein the GAGs are, or have been, subjected to a processing step to obtain the disaccharide units for analysis.

20. The method of claim 18, wherein said at least one processing step does not comprise contacting said sample with a proteolytic agent.

21. The method of claim 1, wherein said sample has been obtained from said subject and has been subjected to processing prior to determining said level and/or chemical composition,
wherein said processing
(a) comprises fragmenting said one or both GAGs into disaccharide units; and
(b) does not comprise prior to (a) at least one of:
(i) contacting said sample with a proteolytic agent; and
(ii) purifying said one or both GAGs in said sample based on the negative charge of said GAGs.

22. The method of claim 21, wherein said method does not comprise the contacting of (b)(i) or the purifying of (b)(ii).

23. The method of claim 21, wherein said fragmenting of (a) is performed by contacting said one or both GAGs with one or more GAG lyase enzymes.

24. The method of claim 23, wherein said one or more GAG lyase enzymes are one or more chondrotinase enzymes and/or one or more heparinase enzymes.

25. The method of claim 21, wherein said purifying of (b)(ii) is performed using an anion-exchange resin.

26. The method of claim 1, wherein said level or chemical composition of said GAG or GAG property is determined by HPLC and mass spectrometry.

27. The method of claim 26, wherein said HPLC is ultra-HPLC.

28. The method of claim 26, wherein said mass spectrometry is triple-quadropole mass spectrometry.

29. The method of claim 1, wherein said subject is a subject at risk of developing cancer, or at risk of the occurrence of cancer, or is a subject having or suspected of having cancer.

30. The method of claim 1, wherein said cancer is
(i) a stage I cancer, a stage II cancer, a stage Ill cancer, a stage IV cancer and a cancer of an unspecified stage; or
(ii) selected from the group consisting of a genitourinary cancer (e.g. kidney cancer, prostate cancer or bladder cancer), a respiratory tract cancer (e.g. lung cancer), a brain tumor, a blood cancer (e.g. a lymphoma), colorectal cancer, uterine cancer, a gastrointestinal-neuroendocrine tumour, a breast cancer, ovarian cancer and head and neck cancer; or
(iii) selected from the group consisting of: bladder cancer, breast invasive ductal carcinoma, cervix squamous cell carcinoma, chronic lymphoid leukaemia, colorectal cancer, endometrial cancer, diffuse glioma, a gastro-intestinal endocrine tumour, head and neck squamous cell carcinoma, diffuse large B-cell lymphoma, non-small cell lung cancer, ovarian cancer, prostate cancer and renal cell cancer.

31. The method of claim 1, wherein said method is used for diagnosing cancer, for the prognosis of cancer, for predicting the occurrence of cancer, for estimate the risk of the occurrence of cancer, for monitoring subjects at risk of the occurrence of cancer, for monitoring the progression of cancer in a subject, for determining the clinical severity of cancer, for predicting the response of a subject to therapy or surgery for cancer, for determining the efficacy of a therapeutic or surgical regime being used to treat cancer, for detecting the recurrence of cancer, for predicting the tissue of origin of a cancer, or for distinguishing small masses suspicious of cancer from other non malignant diseases.

32. A method of screening for cancer in a subject, said method comprising determining the level and/or chemical composition of one or both of the glycosaminoglycans (GAGs) chondroitin sulfate (CS) and heparan sulfate (HS) in a body fluid sample,
wherein said sample has been obtained from said subject and has been subjected to processing prior to determining said level and/or chemical composition, wherein said processing
(a) comprises fragmenting said one or both GAGs into disaccharide units; and
(b) does not comprise prior to (a) at least one of:
(i) contacting said sample with a proteolytic agent; and
(ii) purifying said one or both GAGs in said sample based on the negative charge of said GAGs.

33. The method of claim 32, wherein said method does not comprise the contacting of (b)(i) or the purifying of (b)(ii).

34. (canceled)

35. A method of determining the level and/or chemical composition of the protein-free fraction of one or both of the glycosaminoglycans (GAGs) chondroitin sulfate (CS) and heparan sulfate (HS), said method comprising:
(a) obtaining a body fluid sample from a human patient; and
(b) detecting the level and/or chemical composition of the protein-free fraction one or both of the glycosaminoglycans (GAGs) chondroitin sulfate (CS) and heparan sulfate (HS) in said sample.

36. A method of determining the level and/or chemical composition of one or both of the glycosaminoglycans (GAGs) chondroitin sulfate (CS) and heparan sulfate (HS) in a body fluid sample,
wherein said sample has been obtained from said subject and has been subjected to processing prior to determining said level and/or chemical composition, wherein said processing
(a) comprises fragmenting said one or both GAGs into disaccharide units; and
(b) does not comprise prior to (a) at least one of:
(i) contacting said sample with a proteolytic agent; and
(ii) purifying said one or both GAGs in said sample based on the negative charge of said GAGs.
